(19) United States
(12) Patent Application Publication
(10) Pub. No.: US 2026/0043028 A1
(43) Pub. Date: Feb. 12, 2026

(54) TREATMENT OF SPINAL MUSCULAR ATROPHY

(71) Applicants: University of Pittsburgh - Of the Commonwealth System of Higher Education, Pittsburgh, PA (US); Genentech, Inc., South San Francisco, CA (US)

(72) Inventors: Marco CAPOGROSSO, Pittsburgh, PA (US); Mikael John Lars ELIASSON, South San Francisco, CA (US); Genis PRAT ORTEGA, Pittsburgh, PA (US)

(21) Appl. No.: 19/365,705

(22) Filed: Oct. 22, 2025

Related U.S. Application Data
(63) Continuation of application No. PCT/US2024/025547, filed on Apr. 19, 2024.
(60) Provisional application No. 63/461,545, filed on Apr. 24, 2023.
(60) Provisional application No. 63/510,880, filed on Jun. 28, 2023.
(60) Provisional application No. 63/609,235, filed on Dec. 12, 2023.
(60) Provisional application No. 63/550,939, filed on Feb. 7, 2024.

(51) Int. Cl. C12N 15/113; A61N 1/36; A61P 21/00

(52) U.S. Cl. CPC C12N 15/113; A61N 1/36062; A61P 21/00; C12N 2310/11; C12N 2320/31

(57) ABSTRACT

Disclosed herein are methods for treating spinal muscular atrophy in a subject. Particular methods comprise applying a therapeutically effective amount of an electrical stimulus to sensory neurons innervating a body region of the subject with a motor impairment due to the spinal muscle atrophy, wherein application of the electrical stimulus treats the motor impairment due to spinal muscular atrophy in the subject; in conjunction with administering an SMA therapy.

### CROSS-REFERENCE TO RELATED APPLICATIONS

This application is a continuation of International Application No. PCT/US2024/025547, filed internationally on Apr. 19, 2024, which claims the benefit of U.S. Provisional Application No. 63/461,545, filed Apr. 24, 2023; U.S. Provisional Application No. 63/510,880, filed Jun. 28, 2023; U.S. Provisional Application No. 63/609,235, filed Dec. 12, 2023; and U.S. Provisional Application No. 63/550,939, filed Feb. 7, 2024, the entire contents of which are hereby incorporated by reference herein.

### FIELD

The present disclosure relates to methods of treating Spinal Muscular Atrophy (SMA) in a subject by stimulation of sensory afferents in the subject. The present disclosure also relates to methods of treating SMA in a subject by electrical stimulation of the spinal cord in conjunction with SMA therapy.

### BACKGROUND

SMA is a neurodegenerative disease triggered by a genetic mutation in the survival motoneuron 1 (SMN1) gene (Lefebvre et al., 1995. Cell 80 (1): 155-165). Affected motoneurons (MNs; also called “motor neurons”) in SMA patients are less capable of producing sustained firing and can degrade over time, leading to MN death. Strikingly, even though the SMN1 gene is ubiquitously expressed in all MNs, not all muscles are affected. SMA specially affects lower limbs and, in the more severe cases, respiratory function. Experiments in mouse models have shown that the insufficient expression of SMN protein first produces the dysfunction and, in the late stage of the disease, the death of MNs (Le et al., 2005. Human Molecular Genetics 14 (6): 845-57; Avila et al., 2007. J Clinical Investigation. 117 (3): 659-671). In other words, many, if not all, MNs are non-functioning or dead in an SMA patient, especially if the SMA patient is in an advanced stage of the disease. Moreover, even in those muscles that are affected by SMA, not all neurons are dysfunctional (Fletcher et al., 2017. Nat Neuroscience 20 (7): 905-16; Mentis et al., 2011. Neuron 69 (3): 453-67) suggesting that the muscle weakness is produced by the dysfunction of a percentage of MNs rather than by its death. Thus, MN dysfunction and MN death in SMA patients are two independent processes.

The SMN gene has been mapped by linkage analysis to a complex region in chromosome 5q. In humans, this region contains an approximately 500 thousand base pairs (kb) inverted duplication resulting in two nearly identical copies of the SMN gene. SMA is caused by an inactivating mutation or deletion of the telomeric copy of the gene (SMN1) in both chromosomes, resulting in the loss of SMN1 gene function. However, patients retain the centromeric copy of the gene (SMN2), and the copy number of the SMN2 gene in SMA patients generally correlates inversely with the disease severity; i.e., patients with less severe SMA have more copies of SMN2. Nevertheless, SMN2 is unable to compensate completely for the loss of SMN1 function due to alternative splicing of exon 7 caused by a translationally silent C to T mutation in exon 7. As a result, the majority of transcripts produced from SMN2 lack exon 7 (47 SMN2) and encode a truncated SMN protein that has an impaired function and is rapidly degraded.

The SMN protein is thought to play a role in RNA processing and metabolism, having a well characterized function of mediating the assembly of a specific class of RNA-protein complexes termed snRNPs. SMN may have other functions in MNs, however its role in preventing the selective degeneration of MNs is not well established.

In most cases, SMA is diagnosed based on clinical symptoms and by the presence of at least one copy of the SMN1 gene test. However, in approximately 5% of cases SMA is caused by mutation in genes other than the inactivation of SMN1, some known and others not yet defined. In some cases, when the SMN1 gene test is not feasible or does not show any abnormality, other tests such as an electromyography (EMG) or muscle biopsy may be indicated.

Several mouse models of SMA have been developed. In particular, the SMN delta exon 7 (Δ7 SMN) model (Le et al., Hum. Mol. Genet., 2005, 14:845) carries both the SMN2 gene and several copies of the Δ7 SMN2 cDNA and recapitulates many of the phenotypic features of Type 1 SMA. The Δ7 SMN model can be used for both SMN2 expression studies as well as the evaluation of motor function and survival. The C/C-allele mouse model (Jackson Laboratory strain #008714, The Jackson Laboratory, Bar Harbor, ME) provides a less severe SMA disease model, with mice having reduced levels of both SMN2 full length (FL SMN2) mRNA and SMN protein. The C/C-allele mouse phenotype has the SMN2 gene and a hybrid mSMN1-SMN2 gene that undergoes alternative splicing, but does not have overt muscle weakness. The C/C-allele mouse model is used for SMN2 expression studies.

SMA severity ranges from respiratory failure in the neonatal period (type 1-2) to mild muscle weakness noticed in adulthood (type 4). Infantile SMA is the most severe form of this neurodegenerative disorder. Symptoms include muscle weakness, poor muscle tone, weak cry, limpness or a tendency to flop, difficulty sucking or swallowing, accumulation of secretions in the lungs or throat, feeding difficulties, and increased susceptibility to respiratory tract infections. The legs tend to be weaker than the arms and developmental milestones, such as lifting the head or sitting up, cannot be reached. In general, the earlier the symptoms appear, the shorter the lifespan. As the MN cells deteriorate, symptoms appear shortly afterward. The severe forms of the disease are fatal and all forms have no known cure. The course of SMA is directly related to the rate of MN cell deterioration and the resulting severity of weakness. Infants with a severe form of SMA frequently succumb to respiratory disease due to weakness in the muscles that support breathing. Children with milder forms of SMA live much longer, although they may need extensive medical support, especially those at the more severe end of the spectrum. The clinical spectrum of SMA disorders has been divided into the following five groups:
Type 0 SMA (In Utero SMA) is the most severe form of the disease and begins before birth. Usually, the first symptom of Type 0 SMA is reduced movement of the fetus that can first be observed between 30 and 36 weeks of pregnancy. After birth, these newborns have little movement and have difficulties with swallowing and breathing.
Type 1 SMA (Infantile SMA or Werdnig-Hoffmann disease) presents symptoms between 0 and 6 months. This form of SMA is also very severe. Patients never achieve the ability to sit, and death usually occurs within the first 2 years without ventilatory support.
Type 2 SMA (Intermediate SMA) has an age of onset at 7-18 months. Patients achieve the ability to sit unsupported, but never stand or walk unaided. Prognosis in this group is largely dependent on the degree of respiratory involvement.
Type 3 SMA (Juvenile SMA or Kugelberg-Welander disease) is generally diagnosed after 18 months. Type 3 SMA individuals are able to walk independently at some point during their disease course but often become wheelchair-bound during youth or adulthood.
Type 4 SMA (Adult onset SMA). Weakness usually begins in late adolescence in the tongue, hands, or feet, then progresses to other areas of the body. The course of adult SMA is much slower and has little or no impact on life expectancy.

SMA differs from other types of motor impairment, such as the impairments caused by spinal cord injury or stroke, in that non-functioning MNs are the root cause of the impairment due to SMA. The MNs of spinal cord injury or stroke patients have no underlying cellular pathophysiology. Thus, if the MNs of these patients receive an appropriate excitatory input, they are expected to produce a response. In contrast to the MNs of spinal cord injury or stroke patients, the MNs of SMA patients having cellular pathophysiology as a result of the genetic mutation of the SMN1 gene do not respond appropriately to excitatory input because the MNs themselves are dysfunctional or dead. Accordingly, there is no expectation that SCS, which aims to increase excitatory inputs to the spinal MNs, would produce an effect in an SMA patient similar to those effects observed in other conditions treated with SCS, such as spinal cord injury, stroke, and pain, where MNs are still functioning.

Traditional methods for treating motor impairment, such as exercise or physical therapy, alone may not be effective at treating SMA because neither exercise nor physical therapy can increase the firing rate of MNs to recruit muscle cells. The reduced firing rate of MNs in patients with SMA prevents the thorough engagement of muscle cells and causes a decrease in sensory inputs from the central nervous system. Thus, treatment methods to improve MN firing and function, and thereby recruit more muscle cells, are needed to improve the quality of life of patients with SMA. Neurorestorative agents such as Onasemnogene abeparvovec (Zolgensma®), an IV-administered adeno-associated viral vector-based gene therapy that delivers a copy of the SMN1 gene; Nusinersen (Spinraza®), an intrathecally delivered antisense oligonucleotide (ASO) therapy targeting the SMN2 gene; and Risdiplam (Evrysdi®), an oral SMN2-splicing modifier, are available that slow or prevent MN death due to SMA. Onasemnogene abeparvovec, Nusinersen, and Risdiplam are gene therapies designed to treat SMA by increasing the production of SMN proteins. As provided herein, SMA is caused by deletion or mutation of the SMN1 gene resulting in selective degeneration of SMN-deficient MNs. Although human subjects retain several copies of the SMN2 gene, the small amount of functional SMN protein expressed from SMN2 does not fully compensate for the loss of SMN that would have been expressed from the SMN1 gene.

Onasemnogene abeparvovec is a neurorestorative agent, and more specifically, a gene therapy, and more specifically, a recombinant self-complementary AAV9 containing a transgene encoding the human survival motor neuron (SMN) protein, under the control of a cytomegalovirus enhancer/chicken-β-actin hybrid promoter. Intravenous administration of Onasemnogene abeparvovec results in cell transduction and expression of the SMN protein.

Nusinersen is a neurorestorative agent, and more specifically, a gene therapy, and more specifically, an antisense therapy that alters the SMN2 pre-RNA splicing process by inhibiting splicing factors. In particular, Nusinersen binds to a specific sequence in the intron downstream of exon 7 of the SMN2 transcript. This facilitates the integration of exon 7 into the mRNA and thereby enhances full-length SMA protein levels.

Risdiplam is a neurorestorative agent, and more specifically, a gene therapy, and more specifically, a small molecule splicing modifier that increases the inclusion of exon 7 of SMN2 into mRNA that is transcribed from an SMN2 minigene and the inclusion of exon 7 of SMN1 into mRNA that is transcribed from an SMN1 minigene. The minigenes reproduce the alternative splicing reaction of exon 7 of SMN2 and SMN1, which results in exon 7 skipping in the majority of SMN2 and SMN1 transcripts.

These neurorestorative agents and/or gene therapies alone may not be fully effective in all patients and are especially ineffective in restoring motor function. Thus, new therapies targeting motor impairments in SMA patients and improving the effectiveness of current therapies are needed to improve the quality of life of these patients.

### SUMMARY

Provided herein are implementations of a method for treating SMA in a subject. The method includes applying a therapeutically effective amount of an electrical stimulus to sensory neurons innervating a body region of the subject with a motor impairment due to SMA, wherein the electrical stimulus is applied with one or more electrodes controlled by a neurostimulator, and wherein application of the electrical stimulus treats the motor impairment due to SMA in the subject. In some embodiments, the treatment of SMA comprises treatment with neuromuscular stimulation in combination with a neurorestorative agent. In some embodiments, the neurorestorative agent is a gene therapy such as Onasemnogene abeparvovec, Nusinersen, or Risdiplam.

The foregoing and other objects, features, and advantages of the implementations will become more apparent from the following detailed description, which proceeds with reference to the accompanying figures.

### BRIEF DESCRIPTION OF THE FIGURES

FIGS. 1A-1G illustrate various models of SCS potentiation of MN output, according to some embodiments.

FIGS. 2A-2C illustrate graphs describing force potentiation during simulated voluntary brain input, according to some embodiments.

FIG. 3A illustrates an SMA-affected neuron model with various ion channels, wherein the delayed rectifier potassium channels (K-dr) are blocked, according to some embodiments.

FIG. 3B illustrates a MN model with various ion channels, including a specific delayed rectifier potassium channel (Kv2.1), according to some embodiments.

FIGS. 4A-4C illustrate various HUMAC® Norm configurations to test the maximum torque in different joints in SMA patients receiving SCS stimulation of sensory neurons innervating lower limbs, according to some embodiments.

FIGS. 5A-5B illustrate an isokinetic machine (e.g., a HUMAC® Norm isokinetic machine) configured to test hip flexion and knee extension on an SMA patient, respectively, according to some embodiments.

FIG. 6 illustrates a comparison between the locations of the electrodes during the surgery and the locations after the surgery in an SMA patient, according to some embodiments.

FIGS. 7A-7E illustrate various torque measurements over time for two SMA patients, according to some embodiments.

FIGS. 8A-8B illustrate that spinal cord stimulation increases maximum hip flexion during locomotion for an SMA patient, according to some embodiments.

FIGS. 9A-9B illustrate that spinal cord stimulation on the subject only temporarily disrupts balance for an SMA patient, according to some embodiments.

FIG. 10 illustrates that SCS robustly increases the maximum velocity of an SMA patient, according to some embodiments.

FIGS. 11A-11B illustrate end-of-study effects of SCS in hip flexion for an SMA patient, according to some embodiments.

FIGS. 12A-12F illustrate end-of-study improvements in right knee extension for two SMA patients, according to some embodiments.

FIGS. 13A-13C illustrate end-of-study improvements in left knee extension for an SMA patient, according to some embodiments.

FIGS. 14A-14C illustrate end-of-study improvements in right hip flexion for an SMA patient, according to some embodiments.

FIGS. 15A-15C illustrate end-of-study improvements in left hip flexion for a first SMA patient, according to some embodiments.

FIGS. 15D-15F illustrate end-of-study improvements in right hip extension for a second SMA patient, according to some embodiments.

FIG. 16 illustrates manual muscle test scores for an SMA patient by muscle and session with and without stimulation, according to some embodiments.

FIGS. 17A-17C illustrate distances traveled by two SMA patients during a six-minute walk test across different sessions comparing stimulation off v. stimulation on, according to some embodiments.

FIGS. 18A-18C illustrate Hammersmith Functional Motor Scale Expanded scores and Revised Hammersmith Scale scores for two SMA patients, according to some embodiments.

FIGS. 19A-19D illustrate principal component analysis (PCA) results for two SMA patients, according to some embodiments.

FIGS. 20A-20H illustrate short-term improvements of several gait quality variables for two SMA patients when comparing stimulation off v. stimulation on, according to some embodiments.

FIGS. 21A-21D illustrate end-of-study effects on 6-minute walk test gait patterns for two SMA patients, according to some embodiments.

FIGS. 22A-22H illustrate end-of-study improvements of several gait quality variables for two SMA patients, according to some embodiments.

FIGS. 23A-23B illustrate neural activation results of a first patient during an active task, according to some embodiments.

FIGS. 24A-24B illustrate neural activation results of a second patient during an active task, according to some embodiments.

FIGS. 25A-25B illustrate neural activation results of a first patient during a passive task, according to some embodiments.

FIGS. 26A-26B illustrate MN firing rates during maximum voluntary contraction for a SMA patient at different points during and after the study, according to some embodiments.

FIGS. 27A-27B illustrate end-of-study changes in torque measurements for a SMA patient, according to some embodiments.

FIGS. 28A-28B illustrate short-term increases in torque measurements for a SMA patient when comparing stimulation off v. stimulation on, according to some embodiments.

FIGS. 29A-29C illustrate end-of-study changes in MN input resistance for a SMA patient, according to some embodiments.

FIGS. 30A-30C illustrate long-term changes in torque measurements for two SMA patients when comparing SMA therapy treatment (FIGS. 30B and 30C) vs. no SMA therapy treatment (FIG. 30A), according to some embodiments. The SMA therapy administered was Nusinersen (Spinraza®).

FIGS. 31A-31H illustrate long-term changes in several gait quality variables for two SMA patients when comparing SMA therapy treatment (FIGS. 31E-31H) v. no SMA therapy treatment (FIGS. 31A-31D), according to some embodiments. The SMA therapy administered was Nusinersen (Spinraza®).

FIG. 32 illustrates a table of optimal current values and lead configurations for various SMA patients.

FIG. 33 illustrates a table of discomfort scores for each SCS stimulation configuration as provided by various SMA patients.

FIG. 34 illustrates, using web plots, the percentage increase in absolute torques at all evaluated joints over the course of the study for two SMA patients, compared to the pre-study assessment during isometric tasks.

FIG. 35 illustrates the torques generated by a first SMA patient during maximum isometric contraction for a variety of movements.

FIG. 36 illustrates the torques generated by a third SMA patient during maximum isometric contraction for a variety of movements.

FIGS. 37A-37B illustrate a gait cycle profile of joint angles with SCS off, and ROM with and without SCS at various points in time for the three different SMA patients.

FIG. 38 illustrates a table of ROMs of the hip joint and knee joint for three SMA subjects, pre-study vs. post-study and SCS on vs. SCS off. The pre vs. post comparison is at week 4 compared to baseline (or week 3 for SMA01).

FIG. 39 illustrates z-score analyses associated with performing spinal cord fMRI during activity for various SMA patients.

FIG. 40 illustrates, using a web plot, the percentage of change in MEP peak-to-peak amplitude for various SMA patients post example (or week 4 for SMA01) compared to pre-implant during TMS at rest for every participant and every muscle at 100% TMS pulse strength.

FIG. 41 illustrates TMS recruitment curves for a second SMA patient before implant, during week 3 of the study, and after explant.

FIG. 42 illustrates TMS recruitment curves for SMA03 before implant, during week 3 of the study, and after explant.

FIGS. 43A-43C illustrate bar graphs associated with unit number, innervation area, and peak firing rate of single MN discharges from surface EMG signals during isometric maximum voluntary contraction for three SMA patients, as part of the isometric knee extension exercise.

FIG. 44 illustrates a scatter plot representing long-term changes in isometric max torque measurements produced by left and right hip flexion for the SMA patients SMA01 and SMA02. Negative numbers indicate sessions pre-implant, numbers without (−) or (+) indicate sessions during the study (with implant), and numbers with (+) indicate sessions post-explant.

FIG. 45 illustrates a table of the fatigue values for three SMA patients before, during, and after the study, measured in terms of velocity at the last lap of the 6MWT compared to the first lap.

FIG. 46 illustrates a graph of the velocities lap-by-lap for the 6MWT for three SMA patients.

FIG. 47 illustrates a table of distances traveled by three SMA patients during a 6MWT with SCS OFF across different sessions, specifically comparing pre-implant, end-of-study, and follow-up distances. The numbers in parentheses represent changes over baseline.

FIG. 48 illustrates a graph of improvements in the 6MWT for three SMA patients plotted against each patient's HFMSE score at the beginning of the study. The “38” value refers to the initial HFMSE score of SMA02; “49” of SMA03; and “60” of SMA01.

FIGS. 49A-49C illustrate various plots for step height, step length, and gait velocity to provide an overview of gait changes for three SMA patients during the course of the study.

FIG. 50 illustrates TMS recruitment curves for a first SMA patient before implant and at end-of-study.

FIGS. 51A-51B are traces of single unit motoneuron firing rate during a maximum voluntary contraction in isometric conditions for the right knee of SMA03 pre and post study (FIG. 51A) and with stim on vs. stim off (FIG. 51B).

FIGS. 52A-52B provide quantification of the mean peak firing rates across all isometric conditions for pre vs post (FIG. 52A) and stim on vs stim off (FIG. 52B) for each of three SMA patients.

### DETAILED DESCRIPTION

### I. Introduction

Provided herein are methods for treating a subject with SMA. As discussed in detail herein, application of a therapeutically effective amount of an electrical stimulus to sensory neurons innervating a body region of the subject with a motor impairment due to SMA, via one or more electrodes controlled by a neurostimulator, treats the motor impairment due to SMA in the subject. Without being bound by theory, it is believed that application of the electrical stimulus to sensory neurons directly recruits mono- and poly-synaptic excitatory pathways in the spinal cord, which in turn increases the membrane potential and firing rate probability of spinal MNs innervating the body region of the subject with the motor impairment due to SMA. This recruitment of pathways may increase neuroplasticity, allowing the subject to regain motor functions. Further, in some implementations, application of the therapeutically effective amount of the electrical stimulus to the subject over time (for example, for at least 2 hours/day over a period of at least 6 months, or for at least 1 hour/day over a period of at least 1 month) may lead to ion channel remodeling on the MN membrane and a persistent increase in the firing rate probability of spinal MNs and improved motor impairment due, even in the absence of stimulation. In embodiments, the treatment of SMA comprises treatment with neuromuscular stimulation in combination with a neurorestorative agent. In embodiments, the neurorestorative agent is a gene therapy such as Onasemnogene abeparvovec, Nusinersen, or Risdiplam. In some embodiments, the neurorestorative agent is Risdiplam.

Thus, in an embodiment, therapeutic electrical stimuli may be administered to a subject by implanted or transcutaneous placement of electrodes under the control of an implanted or external neurostimulator. The electrodes and neurostimulator together comprise a system to provide SCS to a subject to improve motor impairment due to SMA such as, for example, muscle weakness, muscle control, and/or speech deficits. This system may be used in combination with other SMA therapies, such as targeted motor rehabilitation, to improve patient outcomes.

The application of SCS on SMA patients has produced results that are unexpected given the unique pathophysiology of SMA. As discussed above, SMA differs from other types of motor impairment, such as the impairments caused by spinal cord injury or stroke, in that non-functioning MNs are the root cause of the impairment due to SMA. Accordingly, there is no expectation that SCS, which aims to increase excitatory inputs to the spinal MNs, would produce an effect in an SMA patient similar to those effects observed in other conditions treated with SCS, such as spinal cord injury, stroke, and pain, where MNs are still functioning.

However, as discussed below, significant immediate and end-of-study effects of SCS have been observed in SMA patients treated according to the present disclosure. Unexpectedly, the magnitude of the immediate effects of SCS in SMA patients is at least as dramatic as those observed in patients with stroke and spinal cord injury. Even more remarkable is the magnitude of end-of-study effects observed after only 4 weeks of treatment according to the present disclosure, as discussed in detail below (e.g., measurements associated with MN input resistance, MN firing rate, torques produced at different leg joints, EMG signals, maximum voluntary contraction during knee extension and/or hip flexion, hip flexion during locomotion, balance, sit-to-stand transitions, maximum running speed, range of motion, muscle strength (manual muscle test), gait (6-minute walk test), motor ability (Hammersmith Functional Motor Scale Expanded and Revised Hammersmith Scale tests), and leg circumferences). Specifically, improvements in muscle strength are of such a magnitude that they cannot be attributed to exercise only. For example, in one experiment, left hip flexion in an SMA patient more than doubled, even though the subject did not perform any hip strength training other than walking during the study. The subject's exercise level was unchanged from his exercise level before the study. These changes are also reflected in improvements observed in the clinical outcome tests such as the manual muscle test, the Revised Hammersmith Scale (RHS) test, and the 6-minute walk test. Moreover, the data does not indicate that a plateau has been reached, indicating that longer use of SCS may lead to even greater improvement in SMA patients.

In the 50+ day post-study period after SCS was removed, the long-term effects of SCS varied between SMA patients. Patients who were treated with SCS but not with an SMA therapy, such as a neurorestorative agent to increase SMN protein production (e.g., Onasemnogene abeparvovec, Nusinersen, or Risdiplam), experienced a gradual decline in certain mobility metrics (e.g., measurements associated with torques produced at different leg joints, locomotion, and gait). Conversely, patients who were treated with an SMA therapy in conjunction with SCS experienced no decline in those mobility metrics. Both groups of patients experienced no decline in some clinical outcome metrics (e.g., RHS tests), with one SMA therapy-treated SMA patient even experiencing an increase in the RHS metric despite not having been treated with SCS in 50+ days. Taken together, the data suggests that SMA patients treated with an SMA therapy in conjunction with SCS may experience long-term benefits from SCS for longer than patients who are treated with SCS alone. In some embodiments, the SMA therapy administered in conjunction with SCS is Onasemnogene abeparvovec, Nusinersen, or Risdiplam. In some embodiments, the SMA therapy is Nusinersen or Risdiplam. In still further embodiments, the SMA therapy is Risdiplam.

Administration of SCS according to some embodiments of the present disclosure may artificially increase the activity on the same sensory afferent fibers (e.g., la) that are affected by SMA. This can have immediate (stimulation ON vs stimulation OFF during the study) and long-term effects on SMA patients (stimulation OFF pre-study vs stimulation OFF post-study). Without being bound by any particular theory, SCS may immediately increase the excitatory inputs to MNs and thus their firing rates. This may produce an immediate improvement in motor deficits. In the long-term, the artificial increase in sensory afferent activity may potentiate the affected sensory synapses and it may revert the maladaptive changes in MNs ion-channels thereby improving MN dysfunction that will result in measurable changes of motor function. Thus, SCS may address motor deficits produced by the decreases in the presynaptic activity of sensory afferent fibers.

In some embodiments, SCS may be combined with pharmaceutical interventions designed to stop the disease progression, such as a drug therapy to increase SMN protein production. In embodiments, the drug therapy includes Onasemnogene abeparvovec, Nusinersen, or Risdiplam. In some embodiments, the drug therapy is Nusinersen or Risdiplam. In still further embodiments, the drug therapy is Risdiplam.

### II. Acronyms

CST corticospinal tract
DRG dorsal root ganglion
EMG electromyography
MN motoneuron or motor neuron
SCS spinal cord stimulation
SMA spinal muscular atrophy
SMN1 survival motoneuron 1
TMS transcranial magnetic stimulation

### III. Summary of Terms

Unless otherwise noted, technical terms are used according to conventional usage. As used herein, the term “comprises” means “includes.” Although many methods and materials similar or equivalent to those described herein can be used, particular suitable methods and materials are described below. To facilitate review of the various implementations, the following explanations of terms are provided:

About: As used herein, the term “about” refers to an approximation of a qualitative or quantitative measurement. Whether the measurement is qualitative or quantitative should be clear from its context. With regard to quantitative measurements, “about” refers to plus or minus 5% of a reference value. For example, “about” 100 mA refers to 95 mA to 105 mA.

Dorsal rootlets: A small branch of a root of a sensory neuron that emerges from the posterior spinal cord and travels to the dorsal root ganglion.

Dorsolateral spinal cord: A region on the exterior surface of the spinal cord located between the dorsal midline and the point of entry of the dorsal rootlets into the main cord.

Electrical stimulus: The passing of various types of current selectively through one or more electrodes to a target location in a subject (for example, specific areas of the dorsolateral spinal cord).

Electrode: An electric conductor through which an electric current can pass. An electrode can also be a collector and/or emitter of an electric current. In some implementations, an electrode is a solid and comprises a conducting metal as the conductive layer. Non-limiting examples of conducting metals include noble or refractory metals and alloys, such as stainless steel, tungsten, platinum, iridium, tantalum, titanium, titanium nitride, and niobium. The electrodes can be either interconnected or independently wired.

End-of-study effects: Intermediate-term effects induced by SCS. Also known as “post-study effects.” As used herein, “end-of-study” can refer to effects measured within about 1 week before or after the end of a four-week study, around the time that the SCS electrodes are removed.

Immediate effects: Immediate (e.g., same-day) effects induced by SCS. Also known as “assistive effects.” Experiments are repeated at short time distances, with and without SCS, to measure the immediate/assistive effects of SCS. As used herein, “immediate” can refer to effects measured during a four-week study and relates to the usage of SCS.

Implanting: Completely or partially placing an electrode(s) or device containing the electrode(s) within a subject, for example, using surgical techniques. A device is partially implanted when some of the device reaches, or extends to the outside of, a subject. Implantable electrodes and devices may be implanted epidurally at the spinal cord, such as at the dorsolateral aspect of the spinal cord. An electrode or device can be implanted for varying durations, such as for a short-term duration (e.g., one or two days or less) or for long-term or chronic duration (e.g., one month, six months, one year, or more), as in a daily assistive device.

Long-term effects: Long-term (e.g., over a time span of months+) effects induced by SCS. Also known as “therapeutic effects.” As described herein, “long-term” can refer to effects measured 50+ days after the electrodes are removed. Long-term effects of SCS can be identified based on changes that occur in the intrinsic motor abilities of SCS subjects over time. These changes are indicative of disease modifying effects of SCS and are measured when SCS is off, thereby reflecting true changes in motor control.

Motor impairment: The partial or total loss of function of a body part, for example, legs, feet, arms, hands, fingers, neck, and trunk (e.g., respiratory muscles). Particular motor impairments include loss of muscle strength, partial paralysis (paresis), loss of dexterity (such as hand finger movement), and uncontrollable muscle tone. A subject can exhibit multiple motor impairments as co-morbidities of SMA.

Motor threshold: The minimum thalamic stimulation intensity that can produce a motor output of a given amplitude from a muscle at rest (RMT) or during a muscle contraction (AMT).

Neurostimulator: A current or voltage-controlled electrical stimulation device. A neurostimulator controls the delivery of an electrical pulse, or pattern of electrical pulses, having defined parameters, for example and without limitation, pulse frequency, duration, amplitude, phase symmetry, duty cycle, pulse current, pulse width, and on-time and off-time. The controlled electrical pulse is delivered through one or more electrodes (for example, leadless electrode(s), or electrode(s) located at the end of a lead, a thin insulated wire) configured to apply the electrical stimulus to target tissue of a subject. A neurostimulator may comprise at least one multiple contact lead. Neurostimulators may be utilized to apply a series of electrical pulse stimuli (e.g., charge balanced pulses) through at least one electrode; for example and without limitation, low-frequency pulse train patterns, frequency-sequenced pulse burst train patterns (e.g., wherein different sequences of modulated electrical stimuli are generated at different burst frequencies), and phasic train patterns (e.g., wherein the stimulus control parameters change over the course of feedback from a subject's movement).

Perceptual threshold: The minimum electrical stimulation intensity necessary for a conscious human to be aware of a particular sensation caused by the electrical stimulation.

Sensory neurons: Also known as afferent neurons, sensory neurons are nerve cells within the peripheral nervous system responsible for converting stimuli from the environment of the neuron into internal electrical impulses and transmitting the impulse to the central nervous system.

SMA therapy: A neurorestorative agent, drug therapy, and/or gene therapy to increase SMN protein production (e.g., Onasemnogene abeparvovec, Nusinersen, or Risdiplam). The SMA therapy may be administered to a subject before, during, and/or after administering stimulation to the subject. In some particular embodiments, the SMA therapy is Nusinersen or Risdiplam, or still more specifically Risdiplam.

Spinal muscular atrophy (SMA): A disease typically caused by an inactivating mutation or deletion in the SMN1 gene on both chromosomes, resulting in a loss of SMN1 gene function.

Subject: Living multi-cellular vertebrate organisms, a category that includes human and non-human mammals, including non-human primates, rats, mice, guinea pigs, cats, dogs, cows, horses, and the like. Thus, the term “subject” includes both human and veterinary subjects. The term “patient” may be used interchangeably with the term “subject.”

Therapeutically effective amount: An amount of a compound or treatment (or both) sufficient to provide a beneficial, or therapeutic, effect to a subject or a given percentage of subjects. Therapeutically effective amounts of a particular compound or treatment can be determined in many different ways, such as assaying for a reduction in a disease or condition (such as motor impairment due to SMA). Therapeutic compounds and treatments can be administered in a single application, or in several applications (e.g., chronically over an appropriate period of time). However, the effective amount can be dependent on the source applied, the subject being treated, the severity and type of the condition being treated, and the manner of administration.

Transcutaneous placement: Placing an electrode(s) or device containing the electrode(s) on or near the skin surface of a subject, for example, using non-invasive techniques. Electrodes may be applied to the skin surface of the subject to apply electrical stimulation, for example, under the control of an external neurostimulator. An electrode or device can be placed transcutaneously for varying durations, such as for a short-term duration (e.g., one or two days or less) or for long-term or chronic duration (e.g., one month, six months, one year, or more), as in a daily assistive device.

Treating/Treatment: With respect to a disease or condition (e.g., SMA), either term includes one or more of (1) preventing the disease or condition, e.g., causing the clinical symptoms of the disease or condition not to develop in a subject that may be exposed to or predisposed to the disease or condition but does not yet experience or display symptoms of the disease or condition, (2) inhibiting the disease or condition, e.g., arresting the development of the disease or condition or its clinical symptoms, and (3) relieving the disease or condition, e.g., causing regression of the disease or condition or its clinical symptoms.

More particularly, the treating or treatment of SMA denotes at least one or more of the following beneficial effects: a reduction in the loss of muscle strength, an increase in muscle strength, a reduction in muscle atrophy, a reduction in the loss of motor function, a reduction in contractures, an increase in MNs, a reduction in the loss of MNs, protection of SMN deficient MNs from degeneration, an increase in motor function, an increase in pulmonary function, a reduction in the loss of pulmonary function, and/or an increase in quality of life.

In further detail, treating or treatment of SMA refers to the functional ability or retention of the functional ability for a human infant or a human toddler to perform certain movements such as to sit up unaided, or for a human infant, a human toddler, a human child or a human adult to perform certain movements such as to stand up unaided, to walk unaided, to run unaided, to breathe unaided, to turn during sleep unaided, or to swallow unaided.

### IV. Stimulation of Sensory Neurons to Treat SMA

Provided herein are methods for treating a subject (for example, a human subject) with SMA. The methods may be utilized to treat (i.e., prevent, ameliorate, suppress, and/or alleviate) a motor impairment due to SMA in the subject. The method includes application of a therapeutically effective amount of an electrical stimulus to sensory neurons innervating a body region of the subject with a motor impairment due to SMA. The electrical stimulus may be applied, for example, with one or more electrodes controlled by a neurostimulator.

In an embodiment, applying the electrical stimulus to the sensory neurons increases the firing rate probability of spinal MNs innervating the body region of the subject with the motor impairment due to SMA. Without being bound by theory, it is believed that application of the electrical stimulus to sensory neurons may directly recruit mono- and poly-synaptic excitatory pathways in the spinal cord, which may indirectly increase the membrane potential and firing rate probability of spinal MNs innervating the body region of the subject with the motor impairment due to SMA. This recruitment of pathways may increase neuroplasticity, allowing the subject to regain motor functions.

Any appropriate subject with, or at risk of, a motor impairment due to SMA can be treated with the methods provided herein. The motor impairment can be in a limb of the upper or lower body, such as above or below the elbow, or above or below the knee, or including the entire arm or leg. The subject can have any of SMA types 1-4, such as type 1, type 2, type 3, or type 4. In some implementations, a subject with SMA is selected for treatment. The method can be in initiated at any time post-onset of the motor impairment in the subject, or even in advance of detectable motor impairment in an SMA patient at risk of motor impairment.

Application of the therapeutically effective amount of the electrical stimulus to the subject with SMA treats at least one motor impairment due to SMA in the subject. For example, application of the therapeutically effective amount of the electrical stimulus can result in a reduction in the loss of muscle strength, an increase in muscle strength, a reduction in muscle atrophy, a reduction in the loss of motor function, an increase in motor function, an increase in pulmonary function, and/or a reduction in the loss of pulmonary function.

In some embodiments, the motor impairment includes reduced control of a limb (such as an arm or leg) and the method provided herein increases control of the limb of the subject by at least 20% (such as at least 30%, at least 40%, at least 50%, at least 60%, at least 70%, at least 80%, at least 90%) relative to before the treatment as measured by any appropriate evaluation metric, such as a balance or strength metric (e.g., a sensory organization test).

In some embodiments, the motor impairment includes reduced postural balance and stability and the method provided herein increases postural balance and stability of the subject by at least 20% (such as at least 30%, at least 40%, at least 50%, at least 60%, at least 70%, at least 80%, at least 90%) relative to before the treatment as measured by any appropriate evaluation metric, such as a balance or strength metric (e.g., a sensory organization test).

In some embodiments, the motor impairment includes reduced leg torque and the method provided herein increases leg torque of the subject by at least 20% (such as at least 30%, at least 40%, at least 50%, at least 60%, at least 70%, at least 80%, at least 90%) relative to before the treatment as measured using a HUMAC® Norm system.

The one or more electrodes may be placed in any suitable position for applying the electrical stimulus to sensory neurons in the subject. In some implementations, the one or more electrodes are positioned to deliver an electrical stimulus to one or more sensory neurons innervating the body region with the motor impairment of the subject.

The spinal cord stimulation (SCS) system may comprise a neurostimulator and one or more spinal leads comprising a plurality of electrodes or contacts. Contacts of the SCS system can target specific muscles. For example, a contact located near the spinal segment L2 may target the hip flexor muscles, whereas a contact located near the spinal segment S1 may target the gluteus muscle and the ankle extensors. Accordingly, the location of the contacts or electrodes can be chosen to selectively target specific nerves innervating the muscles affected in SMA. For example, if a human subject has significant deficits in knee extensors and hip flexors, the stimulation system can be located to selectively target these muscles. In addition, it may be advantageous or desired to not stimulate certain nerves, in order to avoid adverse events. For example, one or more electrodes may be implanted which encompass multiple nerves, including those that innervate muscles which do not have motor impairment or have very low levels of motor impairment. Stimulating muscles that do not have or have low levels of motor impairment may be uncomfortable or harmful. Accordingly, in some embodiments, the number of sensory neurons that are stimulated is less than the number encompassed by the electrode implant. That is, one or more electrodes are implanted in the subject spanning multiple sensory neurons, but not all of those sensory neurons are stimulated.

In some embodiments, the location for administering SCS can be optimized and fine-tuned by stimulating various contact points in an SMA patient, measuring electrical activity, such as electromyographic (EMG) signals, produced by the muscles (e.g., agonist and antagonist muscles of each joint), and evaluating the measurements. In one example, during surgery, the positions of the electrodes are determined by stimulating each contact point and recording EMG signals of muscles ranging from the trunk down to the ankles. Stimulation of a specific contact may begin at a low frequency (e.g., about 1 Hz) and progressively increase the amplitude of the stimulation until electrical activity associated with muscle activity in response to the stimulation is recorded. Based on the recorded electrical activity, the specific muscle(s) targeted by the specific contact can be identified, and thus, the stimulated contact points can be identified as suitable locations for the electrodes. The peak-to-peak amplitude of the EMG waveforms produced due to stimulation may be used to identify responses in the target muscles and thereby determine the correct locations of the muscles. For example, rostral contacts may produce their first waveforms in the hip flexors, whereas caudal contacts may produce their first waveforms in the calf muscles. Stimulations may be repeated until a location is identified where the electrodes activate, for example, from the hip muscles with the most rostral contacts to the calf muscles with the most caudal contacts. In other words, once the multiple contacts of the SCS system provide full coverage of the leg muscles (e.g., from hip to ankle) for the purpose of administering SCS, the position of the lead can be fixed. In some embodiments, the identified location can be used in a second SMA patient exhibiting similar symptoms without performing the above test on the second SMA patient. In some embodiments, the above test is performed during the surgery for implanting the electrodes.

In some implementations, the body region of the subject with the motor impairment is selected from the lower back, hip, leg, ankle, and foot. In such implementations, the one or more electrodes are positioned to apply an electrical stimulus to sensory neurons such as those of the T11-S1 nerve roots. For example, the one or more electrodes may be implanted at the sensory nerve or DRG, or implanted epidurally at dorsal rootlets or at the dorsolateral aspect of the spinal cord for sensory neurons of one or more of the T11-S1 nerve roots. In some embodiments, one or more electrodes are positioned to apply an electrical stimulus to sensory neurons such as those of the L1-T11 nerve roots. In some embodiments, the electrical stimulus is applied to less than the neurons that are covered by the positioning of the one or more electrodes. Thus, for example, in some embodiments, an electrical stimulus is applied to the T11-S1 nerve roots, or a subset therein, such as the T12-S1, L1-S1, L2-S1, L3-S1, L4-S1, L5-S1, T11-L5, T12-L5, L1-L5, L2-L5, L3-L5, L4-L5, T11-L4, T12-L4, L1-L4, L2-L4, L3-L4, T11-L3, T12-L3, L1-L3, L2-L3, T11-L2, T12-L2, L1-L2, T11-L1, or T12-L1 nerve roots. In some embodiments, the electrical stimulus is applied to the L1-S2 nerve roots, or a subset thereof, such as the L1-S1, L1-L5, L1-L4, L1-L3, L2-S2, L3-S2, L4-S2, L5-S2, L2-S1, L2-L5, or L2-L4 nerve roots. In certain embodiments, the electrical stimulus is applied to the L1-S2 nerve roots. In some embodiments, two or more nerve roots independently selected from the group consisting of T11, T12, L1, L2, L3, L4, L5, and S1 are stimulated. In some embodiments, three or more nerve roots independently selected from the group consisting of T11, T12, L1, L2, L3, L4, L5, and S1 are stimulated. In some embodiments, four or more nerve roots independently selected from the group consisting of T11, T12, L1, L2, L3, L4, L5, and S1 are stimulated. In some embodiments, the subject is also administered an SMA therapy, such as Onasemnogene abeparvovec, Nusinersen, or Risdiplam. In some embodiments, the SMA therapy is Nusinersen or Risdiplam. In still further embodiments, the SMA therapy is Risdiplam.

In some implementations, the body region of the subject with the motor impairment is selected from the upper arm, shoulder, arm, hand, and respiratory muscles (such as intercostal muscles or diaphragm). In some embodiments, the muscles are selected from the deltoids, biceps, triceps and wrist extensors flexors of the arms. In such implementations, the one or more electrodes are positioned to apply an electrical stimulus to sensory neurons such as those of the C3-T2 nerve roots. For example, the one or more electrodes may be implanted at the sensory nerve or DRG, or implanted epidurally at dorsal rootlets or at the dorsolateral aspect of the spinal cord for sensory neurons of one or more of the C3-T2 nerve roots.

In some implementations, the body region of the subject with the motor impairment is selected from the upper arm, shoulder, arm, hand, and respiratory muscles (such as intercostal muscles or diaphragm). In some implementations, the body region of the subject with the motor impairment is selected from the upper arm, shoulder, arm, and hand. In some embodiments, the muscles are selected from the deltoids, biceps, triceps and wrist extensors flexors of the arms. In certain embodiments, respiratory muscles (such as intercostal muscles or diaphragm) are specifically not stimulated. In such implementations, the one or more electrodes are implanted in the epidural space of the C4-T1 spinal vertebra to apply an electrical stimulus to one or more sensory neurons present in that space. In some embodiments, an electrical stimulus is applied to the C4-T1 nerve roots or a subset thereof, such as the C4-C8, C4-C7, C4-C6, C4-C5, C5-T1, C5-C6, or C6-T1 nerve roots. In some embodiments, two or more nerve roots independently selected from the group consisting of C4, C5, C6, C7, C8, and T1 are stimulated. In some embodiments, three or more nerve roots independently selected from the group consisting of C4, C5, C6, C7, C8, and T1 are stimulated. In some embodiments, the subject is also administered an SMA therapy, such as Onasemnogene abeparvovec, Nusinersen, or Risdiplam. In some embodiments, the SMA therapy is Nusinersen or Risdiplam. In still further embodiments, the SMA therapy is Risdiplam.

In some implementations, the body region of the subject with the motor impairment is selected from the chest, chest wall, abdomen, upper back, and middle back. In such implementations, the one or more electrodes are positioned to apply an electrical stimulus to sensory neurons such as those of the T3-T10 nerve roots. For example, the one or more electrodes may be implanted at the sensory nerve or DRG, or are implanted epidurally at dorsal rootlets or at the dorsolateral aspect of the spinal cord, for sensory neurons of the T3-T10 nerve roots. In some embodiments, an electrical stimulus is applied to the T3-T10 nerve roots or a subset thereof, such as the T3-T9, T3-T8, T3-T7, T3-T6, T3-T5, T3-T4, T4-T10, T4-T9, T4-T8, T4-T7, T4-T6, T4-T5, T5-T10, T5-T9, T5-T8, T5-T7, T5-T6, T6-T10, T6-T9, T6-T8, T6-T7, T7-T10, T7-T9, T7-T8, T8-T10, T8-T9, or T9-T10 nerve roots. In some embodiments, two or more nerve roots independently selected from the group consisting of T3, T4, T5, T6, T7, T8, T9, and T10 are stimulated. In some embodiments, three or more nerve roots independently selected from the group consisting of T3, T4, T5, T6, T7. T8, T9, and T10 are stimulated. In some embodiments, four or more nerve roots independently selected from the group consisting of T3, T4, T5, T6, T7, T8, T9, and T10 are stimulated. In some embodiments, the subject is also administered an SMA therapy, such as Onasemnogene abeparvovec, Nusinersen, or Risdiplam. In some embodiments, the SMA therapy is Nusinersen or Risdiplam. In still further embodiments, the SMA therapy is Risdiplam.

In some embodiments, the body region of the subject with the motor impairment is selected from the trunk, back, and upper limb. This may encompass, for example, the chest, chest wall, abdomen, upper back, middle back, upper arm, shoulder, arm, and hand. In some embodiments, the muscles are selected from the deltoids, biceps, triceps and wrist extensors flexors of the arms. In certain particular embodiments, one or more electrodes are implanted into the epidural space of the C4 to T1 spinal vertebra, to apply an electrical stimulus to one or more sensory neurons present in that space. For example, in some embodiments one or more of the C5-T1 nerve roots is stimulated, such as the C5-C6, C5-C7, C5-C8, C6-T1, C6-C7, C6-C8, C7-T1, or C7-C8, C8-T1. In some embodiments, C5-T2 nerve roots are stimulated, such as the C5-T2, C6-T2, C7-T2, C8-T2, or T1-T2. In some embodiments, two or more nerve roots independently selected from the group consisting of C4, C5, C6, C7, C8, T1, and T2 are stimulated. In some embodiments, two or more nerve roots independently selected from the group consisting of C5, C6, C7, C8, T1, and T2 are stimulated. In some embodiments, two or more nerve roots independently selected from the group consisting of C5, C6, C7, C8, and T1 are stimulated. In some embodiments, for each electrode, the stimulation amplitude is between 0.2 and 10 mA; stimulation frequency is between 0.1 and 500 Hz, and pulse width is between 100 and 400 μs. In some embodiments, the subject is also administered an SMA therapy, such as Onasemnogene abeparvovec, Nusinersen, or Risdiplam. In some embodiments, the SMA therapy is Nusinersen or Risdiplam. In still further embodiments, the SMA therapy is Risdiplam.

In some embodiments, the placement of the electrodes during surgery can be adjusted to account for movement of the electrodes post-surgery. Such post-surgery movement may be variable and difficult to avoid with non-permanent implants. For example, the electrodes may move caudally and shift medially. Accordingly, based on predicted post-surgery movement of the electrodes, the electrodes can be implanted at adjusted locations during surgery such that, after the surgery, the electrodes would move to the predefined optimal locations. Additional details on the equipment and surgical procedures that can be used to acquire chronic electromyographic (EMG) recordings from leg muscles and to implant targeted spinal cord stimulation systems can be found in Capogrosso et al., 2018. Nature Protocols 13. 2031-2061, which is incorporated by reference herein.

Any suitable stimulation pattern may be used to treat the motor impairment in the subject. In some implementations, the electrical stimulus includes electrical pulses defined by parameters including, for example and without limitation, amplitude, pulse width, and pulse frequency. Such electrical pulses may include charge-balanced pulses, such as cathodic-first biphasic or monophasic charge balanced pulses. In these and further implementations, the electrical stimulus may be a continuous electrical stimulus, or a periodic stimulus.

The stimulation parameters of the SCS can be configured according to the techniques and values/ranges described herein. In one example, the electrical stimulus can be configured to comprise electrical pulses having an amplitude of about 10 μA to about 10 mA, a width between about 40 μs and about 2 ms, and/or a frequency of about 10 Hz to about 2000 Hz. In one example, the electrical stimulus can be configured to have a preferred frequency of about 40 Hz. In some embodiments, for each electrode, the stimulation amplitude is between 0.2 and 10 mA; stimulation frequency is between 0.1 and 500 Hz, and pulse width is between 100 and 400 μs.

In particular examples, the electrical stimulus includes electrical pulses with an amplitude of about 10 μA to about 50 mA, such as about 10 μA to 10 mA, about 10 μA to about 1 mA, about 10 μA to about 100 μA, or about 100 μA to about 1 mA.

In particular examples, the electrical stimulus includes electrical pulses with pulse widths between about 40 μs and about 2 ms; for example, between 40 μs and 2 ms, between 100 μs and 2 ms, between 200 μs and 2 ms, between 300 μs and 2 ms, between 400 μs and 2 ms, between 500 μs and 2 ms, between 600 μs and 2 ms, between 700 μs and 2 ms, between 800 μs and 2 ms, between 800 μs and 2 ms, between 900 μs and 2 ms, between 1 ms and 2 ms, between 1.5 ms and 2 ms, between 80 μs and 1.5 ms, between 100 μs and 1.5 ms, between 200 μs and 1.5 ms, between 300 μs and 1.5 ms, between 400 μs and 1.5 ms, between 500 μs and 1.5 ms, between 600 μs and 1.5 ms, between 700 μs and 1.5 ms, between 800 μs and 1.5 ms, between 800 μs and 1.5 ms, between 900 μs and 1.5 ms, between 1 ms and 1.5 ms, between 1.5 ms and 2 ms, between 80 μs and 1 ms, between 100 μs and 1 ms, between 200 μs and 1 ms, between 300 μs and 1 ms, between 400 μs and 1 ms, between 500 μs and 1 ms, between 600 μs and 1 ms, between 700 μs and 1 ms, between 800 μs and 1 ms, between 800 μs and 1 ms, and between 900 μs and 1 ms.

In particular examples, the electrical stimulus includes a pulse frequency between about 10 Hz and about 2000 Hz; for example, between 20 Hz and 100 Hz, between 20 Hz and 90 Hz, between 20 Hz and 80 Hz, between 20 Hz and 70 Hz, between 20 Hz and 60 Hz, between 20 Hz and 50 Hz, between 20 Hz and 40 Hz, and between 20 Hz and 30 Hz.

In some implementations, the electrical stimulus includes electrical pulses having an amplitude of 10 μA to about 50 mA, a pulse width of between about 40 μs and about 2 ms, and a pulse frequency between about 10 Hz and about 2000 Hz. In some implementations, the electrical stimulus includes electrical pulses having an amplitude of 10 μA to about 10 mA, a pulse width of between about 40 μs and about 2 ms, and a pulse frequency between about 10 Hz and about 1000 Hz. In some implementations, the electrical stimulus includes electrical pulses having an amplitude of 100 μA to about 10 mA, a pulse width of between about 40 μs and about 500 μs, and a pulse frequency between about 10 Hz and about 1000 Hz.

In some implementations, bipolar and/or tripolar stimulation may be used. In some implementations, e.g., to target the right leg muscles, the spinal cord is stimulated using bipolar stimulation of the two most rostral contacts of the right lead. In some examples, the electrical stimulus includes electrical pulses having an amplitude of about 2 mA, a pulse width of about 400 μs, and a pulse frequency of about 40 Hz.

In some implementations, e.g., to target the left leg muscles, tripolar stimulation is used. In some examples, the electrical stimulus includes electrical pulses having an amplitude of about 3.7 mA, a pulse width of about 400 μs, and a pulse frequency of about 40 Hz.

In some implementations, the electrical stimulus includes electrical pulses having an amplitude of less than about 10 mA, a pulse width of between about 80 μs and about 2 ms, and a pulse frequency between about 20 Hz and about 100 Hz. In some examples, the electrical stimulus includes electrical pulses having an amplitude of between 0.5 mA and 5 mA, pulse widths between 80 μs and 200 μs, and a pulse frequency between 20 Hz and 80 Hz. For example, the electrical stimulus may include electrical pulses having an amplitude of between about 1.5 and about 3.5 mA, pulse widths between about 100 μs and about 200 μs, and a pulse frequency between about 40 Hz and about 80 Hz.

The electrical stimulus may be applied to the subject for any suitable amount of time needed to achieve a positive functional benefit for the patient. In some implementations, the electrical stimulus comprises a stimulation pattern of a series of 2 to 5 pulses separated by inter-pulse intervals of about 3 ms to about 10 ms and wherein the series is repeated at a frequency of about 10 Hz to about 100 Hz. In some implementations, the electrical stimulus is applied for at least 1 hour/day over a period of at least 1 month. In some implementations, the electrical stimulus is applied for at least 2 hours/day over a period of at least 6 months.

In particular implementations, the stimulation is applied at or below the subject's motor threshold and/or at or below the subject's perceptual threshold. For example, the stimulation may be applied below the motor threshold and below the perceptual threshold; below the motor threshold, but at or above the perceptual threshold; or below the perceptual threshold, but at or above the motor threshold.

The SCS system can be configured within a few days to provide electrical spinal cord stimulation protocols that allow control over the degree of extension and flexion of muscles during motion of that limb, e.g., each leg during locomotion and real-time processing of gait kinematics and locomotor performance. In some implementations, the subject may be monitored and tested to establish parameters for the electrical stimulation based on the subject's motor disorder and motor impairment; for example, by monitoring one or more motor output(s) that provide a measurement of the extent of the motor impairment and the subject's response to stimulation. In some implementations, electrical stimulation by the electrode(s) is delivered to sensory neurons in the subject while the subject performs a voluntary activity or task affected by the subject's motor impairment; for example, forelimb tasks (e.g., reaching, grabbing, picking with opposable thumbs, grip squeezing, and fine motor tasks involving precision finger movements) or lower limb tasks (e.g., walking, jumping, leg extensions).

Once configured, stimulation bursts are delivered over specific spinal cord locations with precise timing that reproduces the natural spatiotemporal activation of MNs during locomotion. These protocols can also be easily adapted for the safe implantation of systems in the vicinity of the spinal cord and to provide SCS involving real-time movement feedback and closed-loop controllers, as discussed below. In some implementations, the parameters of the electrical stimulus controlled by the neurostimulator are adjusted according to changes in the one or more motor output(s) that are monitored while the subject performs a specific task, for example, so as to improve the motor outputs, thereby treating the subject's motor impairment. In particular implementations, the adjusted neurostimulator is part of a daily assistive device to treat the subject over an extended period of time. In specific implementations, the operation of the device and/or the neurostimulator can be at least partially under the control of the subject once the subject is released from a clinical setting. In these and further implementations, the subject is taught how to use the device and/or the neurostimulator. In some non-limiting implementations, this treatment may increase inputs on the membrane of the spinal MNs by means of direct recruitment of sensory afferents from the electrical pulses and or may lead to ion channel remodeling on the MN membrane to increasing firing rate probability of spinal MNs and/or improving motor impairment due to SMA, including when the electrical stimulus is no longer applied to the patient. Accordingly, the disclosure includes methods of increasing the firing rate of motoneurons impaired by SMA.

Stimulation of sensory afferents using implanted electrodes is an advanced neurosurgical procedure involving the implantation of one or more electrode(s) that deliver an electrical stimulus under the control of an externalized or implanted neurostimulator unit. Implantation of the electrode(s), and/or a neurostimulator in examples where the neurostimulator is not externalized, is typically performed by a clinical team including neurologists, neurosurgeons, neurophysiologists, and other specialists trained in the assessment, treatment, and care of neurological conditions. Typically, following selection of an appropriate subject and determination of the target area of the subject to be stimulated, precise placement of at least one electrode in the area of the patient's sensory afferents (such as the dorsolateral aspect of the spinal cord) is carried out in an operating room setting, typically utilizing spinal cord imaging technology. After administration of local anesthesia, the subject undergoing electrode implantation experiences little discomfort, and may be kept awake during the implantation procedure to allow communication with the surgical team.

Some implementations herein employ an implant that includes one or more electrodes and/or neurostimulator implanted (e.g., fully or partially implanted) in the subject. Further implementations herein employ an implant that includes one or more magnets or optical fibers, and/or a neurostimulator implanted in the subject.

Numerous types and styles of implants (for example, implants including one or more electrodes for providing an electrical stimulus) are available and known to those in the art. Any implant for specific stimulation of sensory neurons in a subject may be utilized in specific implementations. In some implementations, more than one electrode is implanted, such as an array of electrodes. In additional implementations, a device is provided that can include one or more electrodes. Non-limiting examples include, electrode arrays, penetrating microarrays (e.g., Utah and Michigan microarrays), microwire electrodes and arrays, nerve cuffs, and paddle arrays.

In some implementations, the one or more electrodes are implanted at the dorsal rootlets of the one or more sensory neurons innervating the body region with the motor impairment of the subject. In some implementations, the one or more electrodes are implanted at the dorsolateral aspect of the spinal cord adjacent to the dorsal roots of the one or more sensory neurons innervating the body region with the motor impairment of the subject. In some implementations, the one or more electrodes are implanted at the dorsal root ganglia of the one or more sensory neurons innervating the body region with the motor impairment of the subject. In some implementations, the one or more electrodes are contained in a cuff that surrounds or at least partially surrounds a peripheral nerve containing the sensory neurons innervating the body region of the subject with the motor impairment due to SMA. In further implementations, the one or more electrodes penetrate a peripheral nerve or dorsal root ganglion containing the sensory neurons innervating the body region of the subject with the motor impairment due to SMA.

In some implementations, epidural electrical stimulation (EES) targeting the dorsal rootlets using a paddle electrode array is utilized in the disclosed methods, for example, as described in Rowald et al., 2022, Nat. Medicine, 28:260-271 and Wagner et al., 2018, Nature, 563:65-71, each of which is incorporated by reference herein. Additional non-limiting examples of paddle arrays and their use are provided, for example, in US2009/0351221, US2019/0366077, each of which is incorporated by reference herein.

In some implementations, circuitry is implanted connecting a neurostimulator to the one or more electrodes. In particular implementations, the circuits are fully implanted (typically in a subcutaneous pocket within a subject's body) or are partially implanted in the subject. The operable linkage of the neurostimulator to the electrode(s) can be by way of one or more leads, although any operable linkage capable of transmitting a stimulation signal from the circuitry to the electrodes may be used in specific implementations.

Any suitable control system can be used with the electrodes to apply the electrical stimulus to the subject. Non-limiting examples of controllable neurostimulation systems are provided in US2020/0254260, US2020/0360693, US2020/0360697, US2020/0152078, U.S. Pat. Nos. 10,252,065, 10,799,702, US2021/0016093, and US2020/0391030, each of which is incorporated by reference herein. Further, non-limiting examples of closed-loop neurostimulation systems are provided in U.S. Pat. Nos. 10,265,525, 10,279,167, 10,279,177, 10,391,309, 10,751,539, 10,981,004, and US2020/0147382, each of which are incorporated by reference herein.

Post-operative control of selective electrical stimulation by the implanted electrode is provided in some implementations by a neurostimulator that may be externalized or implanted; for example, subcutaneously (e.g., in the chest or belly of the subject). In some implementations, disclosed methods are affected by the use of an implanted neurostimulator that controls the stimulation (e.g., electrical stimulation via one or more implanted electrode(s)) according to predetermined parameters or parameters determined by feedback in a closed-loop system. In particular implementations, the one or more electrodes and the neurostimulator comprise a daily assistive device that improves muscle weakness in an affected limb of the subject.

Following recovery from the implantation, surgery, and connection of electrode leads to the neurostimulator, the subject may be monitored and tested to establish parameters for the electrical stimulation based on the subject's motor disorder and motor impairment; for example, by monitoring one or more motor output(s) that provide a measurement of the extent of the motor impairment and the subject's response to stimulation. In some implementations, electrical stimulation by the implanted electrode(s) is delivered to sensory neurons in the subject while the subject performs a voluntary activity or task affected by the subject's motor impairment; for example, forelimb tasks (e.g., reaching, grabbing, picking with opposable thumbs, grip squeezing, and fine motor tasks involving precision finger movements) or lower limb tasks (e.g., walking, jumping, leg extensions).

In some implementations, a transcutaneous electrical stimulation system is used to apply the electrical stimulus to the sensory neurons in the subject. A non-limiting example of a transcutaneous stimulation system is provided in U.S. Pat. No. 10,806,927, which is incorporated by reference herein. In such transcutaneous implementations, the electrical stimulus may comprise electrical pulses having an amplitude of about 10 μA to about 100 mA, a width between about 40 μs and about 2 ms, and a frequency of about 10 Hz to about 10000 Hz.

As described herein, the subject is also administered an SMA therapy in conjunction with the SCS. In some embodiments, the SMA therapy is Onasemnogene abeparvovec, Nusinersen, or Risdiplam.

In some embodiments, the subject is receiving Onasemnogene abeparvovec. Onasemnogene abeparvovec is also known as ZOLGENSMA®, and is approved for single-dose IV administration only. Thus, wherein a subject is administered an SMA therapy in conjunction with SCS as described herein, and the SMA therapy is Onasemnogene abeparvovec, the single dose of Onasemnogene abeparvovec may have been administered to said subject within 3 months, within 6 months, within 9 months, within 12 months, within 15 months, within 18 months, within 21 months, or within 24 months prior to beginning the SCS. For example, in some embodiments, Onasemnogene abeparvovec was administered at least 3 months, at least 6 months, at least 9 months, at least 12 months, at least 15 months, at least 18 months, at least 21 months, or at least 24 months prior to the initiation of SCS. In some embodiments, the subject was administered Onasemnogene abeparvovec greater than 24 months prior to the initiation of SCS, such as greater than 2 years, greater than 3 years, greater than 4 years, greater than 5 years, or greater than 6 years prior to the initiation of SCS. SCS initiation in this instance is the application of electrical stimulus; the implantation of the one or more electrodes must also occur, but does not constitute beginning or initiating SCS. Further, in some embodiments, initiating SCS comprises the beginning or first instance of electrical stimulation post-implantation (e.g., not including stimulation that may occur during the implantation to position the electrodes or map their stimulus to the nerve roots and/or muscle groups). In some embodiments, initiating SCS comprises the beginning or first instance of electrical stimulation post-implantation, according to a stimulation protocol administered or overseen by a medical doctor. Reference to SCS in these embodiments may also be described as applying electrical stimulus to sensory neurons.

In some embodiments, the subject is receiving Nusinersen. Nusinersen is also known as SPINRAZA™, and in the US is approved for administration intrathecally according to a recommended dosing schedule: 12 mg (5 mL) per administration, initiated with 4 loading doses, the first three of which are at 14-day intervals; and the 4th loading dose at 30 days after the 3^{rd} loading dose; with maintenance dosing once every 4 months thereafter. Accordingly, wherein the subject is administered Nusinersen in conjunction with SCS, the initiation of SCS may occur at any point during the Nusinersen dosing schedule. Thus, in some embodiments, SCS is initiated between the first and second Nusinersen loading doses; between the second and third Nusinersen loading doses; between the third and fourth Nusinersen loading doses; or after the fourth loading dose. Wherein SCS is initiated after the fourth loading dose of Nusinersen, the SCS may be initiated at any point, that is, after the fourth loading dose but before the first maintenance dose, or between any subsequent maintenance doses. SCS initiation in this instance is the application of electrical stimulus; the implantation of the one or more electrodes must also occur, but does not constitute beginning or initiating SCS. Further, in some embodiments, initiating SCS comprises the beginning or first instance of electrical stimulation post-implantation (e.g., not including stimulation that may occur during the implantation to position the electrodes or map their stimulus to the nerve roots and/or muscle groups). In some embodiments, initiating SCS comprises the beginning or first instance of electrical stimulation post-implantation, according to a stimulation protocol administered or overseen by a medical doctor. In some particular embodiments, a subject who is administered SCS as described herein in conjunction with Nusinersen has already finished the four loading doses, and is within the maintenance dosing schedule, when SCS is initiated. In some embodiments, the subject received the first Nusinersen dose (e.g., the first loading dose) at least 58 days before initiating SCS. In some embodiments, the subject received the first Nusinersen (e.g., the first loading dose), at least 58 days, at least 3 months, at least 4 months, at least 6 months, at least 9 months, at least 12 months, at least 16 months, or at least 18 months prior to initiating SCS. In some embodiments, the subject has been on Nusinersen therapy for at least 58 days, at least 3 months, at least 4 months, at least 6 months, at least 9 months, at least 12 months, at least 16 months, or at least 18 months, when beginning SCS. In still yet further embodiments, the subject has been receiving Nusinersen therapy for at least 2 years, at least 3 years, at least 4 years, at least 5 years, at least 6 years, or more, prior to the initiation of SCS. In some embodiments, the subject is on the maintenance dosing schedule of Nusinersen when beginning SCS. Reference to SCS in these embodiments may also be described as applying electrical stimulus to sensory neurons.

In still further embodiments, the subject is receiving Risdiplam. Risdiplam is also known as EVRYSDI®, and is approved for once-daily oral dosing according to the following: for patients less than two months of age, 0.15 mg/kg body weight once daily; for patients from two months to less than 2 years of age, 0.2 mg/kg body weight once daily; for patients two years of age and older weighing less than 20 kg, 0.25 mg/kg body weight once daily; and for patients two years of age and older weighing more than 20 kg, 5 mg once daily. Accordingly, wherein the subject is administered Risdiplam in conjunction with SCS, the initiation of SCS occurs after the subject has begun administration of Risdiplam. In some embodiments, the subject began administration of Risdiplam within 3 months, within 6 months, within 9 months, within 12 months, within 15 months, within 18 months, within 21 months, or within 24 months prior to initiating SCS. In some embodiments, the subject has been administered Risdiplam therapy for greater than 1 year, 2 years, greater than 3 years, greater than 4 years, greater than 5 years, or greater than 6 years prior to the initiation of SCS. SCS initiation in this instance is the application of electrical stimulus; the implantation of the one or more electrodes must also occur, but does not constitute beginning or initiating SCS. Further, in some embodiments, initiating SCS comprises the beginning or first instance of electrical stimulation post-implantation (e.g., not including stimulation that may occur during the implantation to position the electrodes or map their stimulus to the nerve roots and/or muscle groups). In some embodiments, initiating SCS comprises the beginning or first instance of electrical stimulation post-implantation, according to a stimulation protocol administered or overseen by a medical doctor. Reference to SCS in these embodiments may also be described as applying electrical stimulus to sensory neurons.

In particular implementations, methods disclosed herein can be used in combination with protocolled physical rehabilitation exercises to improve long-term outcomes.

### EXAMPLES

The following examples are provided to illustrate particular features of certain implementations, but the scope of the claims should not be limited to those features exemplified.

### Example 1

### Biophysical Model of Spinal Cord Stimulation to Treat SMA

This example provides a biophysical model illustrating the effectiveness of sensory neuron stimulation therapy for SMA patients, as well as stimulation protocols for treating SMA patients (such as type 3 and type 4 SMA patients) with stimulation of the dorsolateral aspect of the spinal cord.

The application of SCS on SMA patients has produced results that are unexpected results given the unique pathophysiology of SMA. Although SMA patients generally have an intact corticospinal tract, unlike patients with spinal cord injury or stroke, many, if not all, MNs are non-functioning or dead in an SMA patient, especially if the SMA patient is in an advanced stage of the disease, making them functionally inert. Accordingly, there is no expectation that SCS, which aims to increase excitatory inputs to the spinal MNs, would produce any effect to an SMA patient because the MNs in the SMA patient are already non-functioning or dead. There is certainly no expectation that the application of SCS to treat SMA would produce the effects observed in other conditions treated with SCS, such as spinal cord injury, stroke, and pain, where MNs are still functioning.

Surprisingly, however, as discussed below, both significant immediate and end-of-study effects of SCS have unexpectedly been observed in SMA patients treated according to the present disclosure. Although experiments with SMA mouse models have shown a decrease in presynaptic activity from sensory afferent fibers, the present disclosure demonstrates that by artificially increasing the activity at the sensory afferent fibers through SCS, a therapeutically effective amount of SCS appropriately applied, such as at the dorsolateral aspect of the spinal cord, may excite the MNs through the remaining excitatory connections and may generate long-term potentiation of the affected synapses that may contribute to reversing the electrical changes in SMA-affected MNs. Moreover, therapeutically effective amount of SCS may also potentiate the firing rate of SMA-unaffected MNs, producing a more robust effect.

### Network Model of the Spinal Cord Circuitry

### Healthy Neuron Model

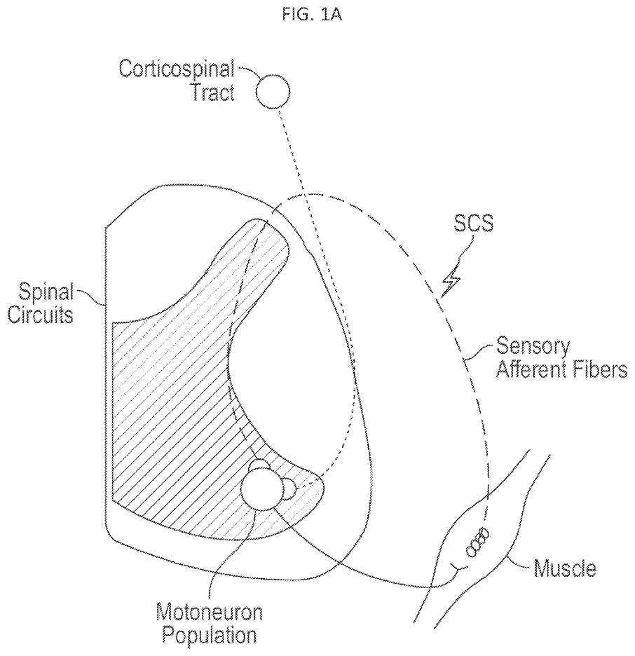

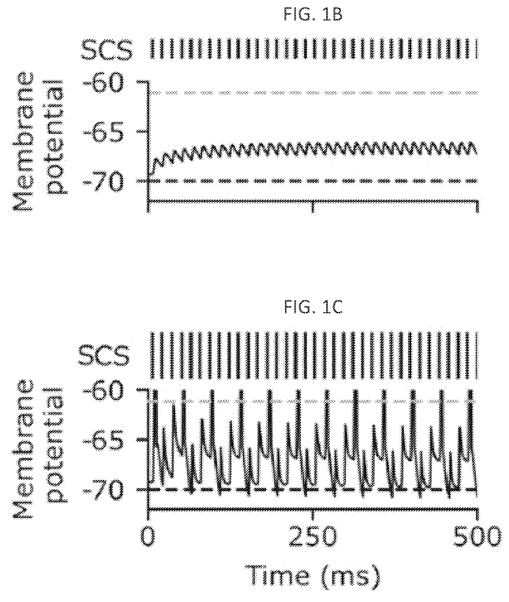

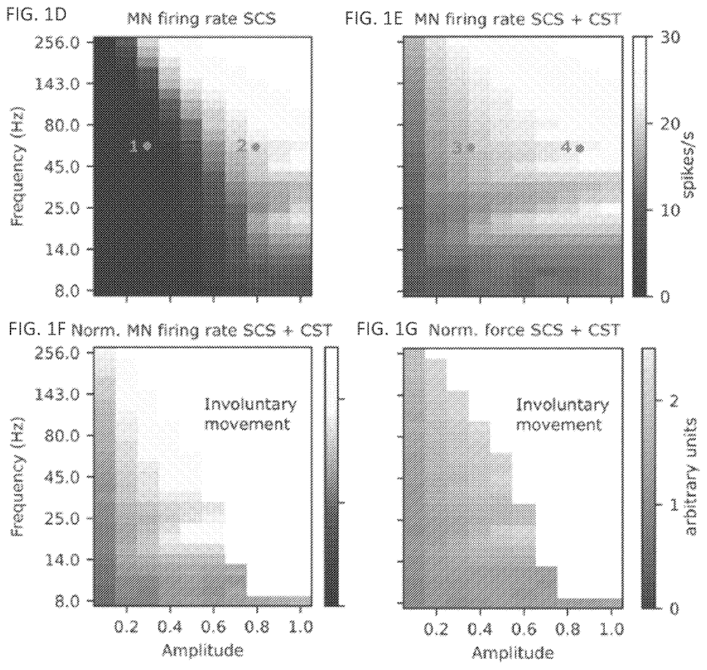

FIG. 1A illustrates a network model diagram, in which MNs receive excitatory inputs from the corticospinal tract and the spinal cord stimulation (SCS), such as dorsolateral SCS, through the recruitment of sensory afferent fibers. As shown in FIG. 1A, a biophysical model was built with a population of healthy MNs (N=169 modified Hodgkin-Huxley neurons, McIntyre et al., 2002. J Neurophysiology 88 (4): 1592-1604). Each MN receives excitatory inputs from the corticospinal tract (CST, N=110) and the SCS through the recruitment of sensory afferent fibers (N=60) (see Capogrosso et al., 2013. J Neuroscience. 33 (49): 19326-40; Gerasimenko et al., 2006. J Neuroscience Methods 157 (2): 253-63; Hofstoetter et al., 2015. J Neurophysiology 114 (1): 400-410; Rattay et al., 2000. Spinal Cord 38 (8): 473-89). The difference between both excitatory sources is that the CST was modeled as a population of Poisson neurons while SCS had its own frequency and amplitude. To investigate the restoration of movement during SCS, the force produced by the MN pool was quantified in arbitrary units (Fuglevand et al., 1993. J Neurophysiology, 70 (6): 2470-2488). In contrast to the CST that represents voluntary inputs from the brain to the MNs, SCS is controlled by the experimenter. Thus, to improve voluntary movement, SCS should potentiate the MNs firing rate only when the CST is active.

### SMA Neuron Model

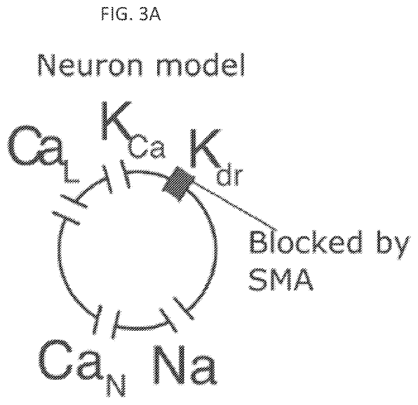

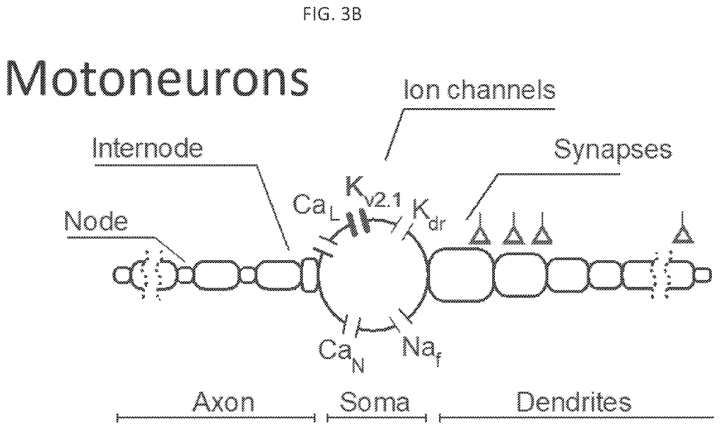

FIG. 3A illustrates an SMA-affected neuron model with various ion channels, wherein the delayed rectifier potassium channels (K-dr) are blocked. For the healthy neuron model, the established approach to describe the dynamics of the membrane potential as a function of the different ion channels is the Hodgkin-Huxley model. In this model, the membrane potential (V) is described by:

$$C \frac{dV}{dt} = {\sum }_{ions} {g}_{x} ( V - {V}_{x} ) + I$$

where C is the membrane capacity, I is current artificially injected to the neuron and g is the conductance x of the ion-channel x that, in general, depends on the membrane potential and ion concentrations. Following previous literature (Booth et al., 1997, J Neurophysiology 78 (6): 3371-85; McIntyre et al., 2002. J Neurophysiology 88 (4): 1592-1604; Moraud et al., 2016. Neuron 89 (4): 814-28), the following ion channels are included: delayed rectifier sodium (Na) and potassium (K-dr), N-like calcium (Ca-N), L-like calcium (Ca-L) and calcium-dependent potassium (K(Ca)), as shown in FIG. 3A. The model is implemented in a neuron stimulation environment designed for modeling individual neurons and networks of neurons referred to herein as NEURON (Hines and Carnevale. 1997. Neural Computation 9 (6): 1179-1209).

FIG. 3B illustrates a MN model with various ion channels, including a specific delayed rectifier potassium channel (Kv2.1). Without being bound by any particular theory, the SMA-induced loss of sensory afferent inputs (e.g., la) may change the Kv2.1 channels of MNs into a dysfunctional state with increased refractory periods (e.g., slower firing rates) and/or increased input resistance. Additionally, deficits in MN function may be reverted by increasing sensory afferent inputs (e.g., via SCS) into SMA-affected MNs, which decreases the input resistance of the MNs. Furthermore, SCS may immediately increase sensory afferent inputs, thereby increasing the firing rates of SMA-affected MNs. Over time, SCS may rescue MN function by changing the Kv2.1 channels of MNs to their healthy, functional state.

SCS Increases the Excitability of MNs without Producing Involuntary Movement

FIGS. 1B-1G illustrate various models of SCS potentiation of MN output. In all panels of FIGS. 1D-1G, the “amplitude” unit is the percentage of sensory afferent fibers recruited by the SCS. FIG. 1B plots the membrane potential (mV) of a MN stimulated with a frequency of 69 Hz and an amplitude that recruited 30% of the sensory afferent fibers. Note that the parameters illustrated in FIG. 1B correspond to the point labeled “1” on FIG. 1D. The black dashed line shows the resting potential of the MN membrane potential, while the gray dashed line shows the MN spike threshold. For the appropriate parameters, SCS can increase the excitability of MNs without generating action potentials. FIG. 1C plots the membrane potential (mV) of a MN stimulated with a frequency of 69 Hz and an amplitude that recruited 80% of the sensory afferent fibers. Note that the parameters illustrated in FIG. 1C correspond to the point labeled “2” on FIG. 1D. FIG. 1D, which illustrates a gradient plot, plots MN firing rate as a function of SCS parameters without input from the corticospinal tract (CST), which shows that SCS by itself produces involuntary movements (MNs firing rate >8 Hz). Greater spikes per second is illustrated by the darker shading in each of FIGS. 1D-IF on a scale of 0-30 spikes/s. FIG. 1E, which illustrates a gradient plot, plots MN firing rate as a function of SCS parameters with input from the CST. FIG. 1F, which illustrates a gradient plot, plots MN firing rate for combinations of SCS parameters that generate only voluntary movement normalized by the MNs firing rate without SCS. FIG. 1G, which illustrates a gradient plot, plots how SCS potentiates the force generated by the MNs population. The “arbitrary units” scale of FIGS. 1F and 1G represent how many times larger the firing rate with SCS is than the firing rate without SCS. The upper end of the “arbitrary units” scale is 2.2 times. Thus, these figures show that MN firing rate is up to 2.2 times larger with SCS than without.

To study the effect of continuous SCS on the MNs firing rate, a range of stimulation parameters was explored. In general, the firing rate of MNs increased as a function of both SCS frequency and amplitude, as shown in FIG. 1D. However, a local maximum at 25 Hz was identified, which is related to time constants of the modified Hodgkin-Huxley neuron model (McIntyre et al., 2002. J Neurophysiology 88 (4): 1592-1604). SCS was able to increase the firing rate of the MNs above the minimum firing rate to produce movement (>8 Hz) (Monster and Chan. 1977. J Neurophysiology 40 (6): 1432-43), as shown in FIGS. 1C and 1D. Any combination of SCS parameters that generated movement without the participation of the CST (i.e., involuntary movement) were discarded for the restoration of voluntary movement. However, SCS was also able to increase MN excitability (i.e., depolarize the membrane potential) without producing involuntary movement (i.e., MN firing rate <8 Hz), as shown in FIGS. 1B and 1D. This increase of the MN excitability represents a mechanism to potentiate CST inputs.

### SCS Potentiates Supraspinal Inputs Increasing MN Firing Rate

### Healthy Neuron Model

Having shown that SCS can increase MN excitability in the healthy model, whether SCS can also potentiate CST inputs to activate MNs and produce force was assessed. To perform this assessment, the input from the CST was fixed to generate a relatively low firing rate (9.3 Hz) in the MNs. The MNs firing rate was computed while systematically varying the SCS parameters (frequency and amplitude). For a range of these parameters, SCS potentiated the inputs from the CST to increase the firing rate of the MNs without generating involuntary movement, as shown in FIGS. 1E and 1F. The enhanced firing rate of the MN with SCS produced a force up to 2.2 times stronger than the firing rate generated by the same CST Inputs without SCS, as shown in FIG. 1G.

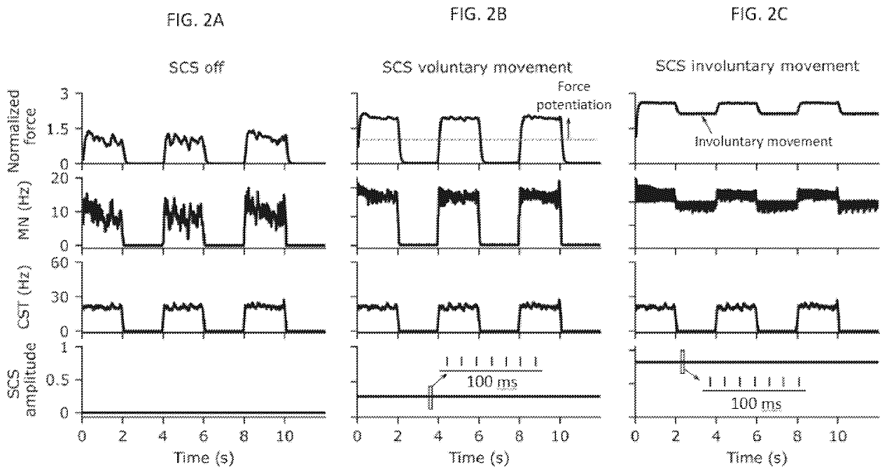

FIGS. 2A-2C illustrate graphs describing force potentiation during simulated voluntary brain input. FIG. 2A plots the following network activity parameters: the SCS amplitude, the firing rate of the corticospinal tract (CST) in Hz, MN firing rate in Hz, and the force normalized by the mean force applied. For the network activity of FIG. 2A, SCS stimulation is not applied, and a CST input of 22 Hz is applied. FIG. 2B plots network activity with SCS applied in the voluntary movement region. Note that the parameters illustrated in FIG. 2B correspond to the point labeled “3” on FIG. 1E. MNs firing rate and thus force are potentiated with respect to SCS off. FIG. 2C plots network activity with SCS applied in the involuntary movement region. Note that the parameters illustrated in FIG. 2C correspond to the point labeled “4” on FIG. 1E. MNs firing rate is high during both phases: with and without inputs from the CST.

To further understand the potentiation of CST inputs driven by SCS, an oscillatory force task was modeled where CST inputs arrived at the MNs in a periodic fashion, as shown in FIGS. 2A-2C. The differences between the voluntary and the involuntary movement regions were assessed. In the voluntary movement region, SCS increased the excitability of MNs producing firing rates higher than those observed without SCS. However, SCS did not increase the firing rate of the MNs during periods without CST inputs, as shown in FIGS. 2A and 2B. In the involuntary movement region, SCS increased the firing rate during the entire trial also during periods without inputs from the CST, as shown in FIG. 2C. Together these results show that, in the biophysical model of a healthy spinal cord, it is possible to potentiate the inputs from the CST to generate enhanced firing rate of the MNs, and thus a stronger force.

### SMA Neuron Model

A series of experiments with an SMA mouse model have shown that SMA affected MNs have dysfunctional electrical properties (Mentis et al., 2011. Neuron 69 (3): 453-67; Fletcher et al., 2017. Nat Neuroscience 20 (7): 905-16) due to a decrease in the synapses' activity from the sensory afferent fibers and a block of delayed rectifier potassium channels (Fletcher et al., 2017. Nat Neuroscience 20 (7): 905-16; Simon et al., J Neuroscience. 41 (2): 376-389, 2021). The downregulation of the K-dr channel, as shown in FIG. 3A, decreases the excitability of the MNs. Thus, to model the SMA-affected MNs, the synaptic weights (i.e., the strength of the synapses) are decreased from the sensory afferent fiber and the conductance of the delayed rectifier potassium channel, as shown in FIG. 3A. This neuron model may be validated by reproducing the following three dysfunctional electrical properties of the SMA-affected MNs:
(1) High input resistance: Within the NEURON simulation environment, which is designed for modeling individual neurons and networks of neurons, it is easy to inject current directly into the MN and compute the input resistance as the slope of the voltage-current function. The block of the potassium channels will decrease the outflux of potassium ions, thereby increasing the input resistance.
(2) Reduced rheobase: As in (1), current is injected into the MN from which it is possible to compute the minimum input current to generate an action potential. Similarly, decreasing the outflux of potassium ions depolarizes the membrane potential faster, decreasing the injected current needed to trigger an action potential.
(3) Low firing rate induced by a current higher than the current needed to induce repeating firing: Injecting an artificial current above the threshold for repeating firing rate of the SMA-affected MN tests whether the block of the potassium channels is enough to reduce the firing rate. Potassium channels open after the action potential to repolarize the membrane potential. With their block the repolarization phase will be slower than in a healthy MN, decreasing the output firing rate (Fletcher et al., 2017. Nat Neuroscience 20 (7): 905-16).

### Example 2

### Spinal Cord Stimulation to Treat SMA

This example describes a particular method that can be used to treat SMA in a patient by applying a therapeutically effective amount of an electrical stimulus to the dorsolateral aspect of the spinal cord containing sensory neurons innervating a body region of the patient with motor impairment due to SMA. Although particular methods and protocols are provided, one skilled in the art will appreciate that variations can be made without substantially affecting the treatment. Three patients with SMA were identified and participated in this spinal cord stimulation trial. One patient (“SMA01”, described in more detail below) was not also being administered an SMA therapy while participating in this SCS trial. Two patients (SMA02 and SMA03, described in further detail below) were being administered the SMA therapy Nusinersen while participating in this SCS trial.

Three human patients with Type 3 SMA that show quantifiable motor deficits of the legs are selected for treatment. The first patient (described hereinafter as “SMA01”) was a 22-year-old man, with an initial Hammersmith Functional Motor Scale Expanded (HFMSE) motor ability score of 60 (out of 66), and he was not receiving SMA therapy. The second patient (described hereinafter as “SMA02”) was a 55-year-old man, with a lower motor ability than SMA01 (as reflected by a HFMSE score of 38 out of 66), and he was receiving SMA therapy (Nusinersen). The third patient (described hereinafter as “SMA03”) was a 30-year-old man whose motor ability falls between those of SMA01 and SMA02 (as reflected by a HFMSE score of 49 out of 66), and he was also receiving SMA therapy (Nusinersen). Percutaneous, bilateral, linear spinal leads were implanted in the three patients near their lumbar spinal cords for a period of up to 29 days (four weeks).

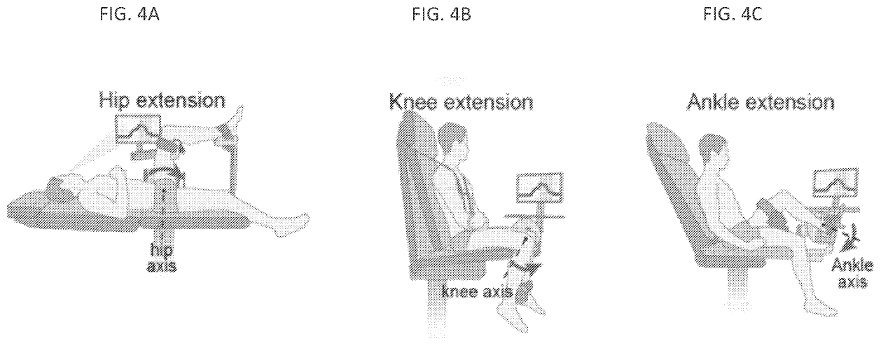

To quantify immediate motor improvement driven by the SCS, maximum torques produced at different joints such as the hip, knee and ankle during isometric movements are measured. The HUMAC® Norm system was used for these measurements. With this system, the patients can be placed in different positions to evaluate the maximum torque of such joints (FIGS. 4A-4C). FIG. 4A illustrates hip extension, FIG. 4B illustrates knee extension, and FIG. 4C illustrates ankle extension.

During the assessment, the patients produced a progressive contraction from rest to maximum strength, where they received real-time torque visual feedback. The same assay was repeated to systematically explore different SCS parameters, including without SCS, to determine parameters that were most effective to reduce motor impairment in the patients. As an example, an electrical stimulus of electrical pulses having an amplitude of about 10 μA to about 50 mA, a width between about 40 μs and about 2 ms, and a frequency of about 10 Hz to about 2000 Hz can be applied. Additionally, the electrical activity produced by the agonist and antagonist muscles of each joint using surface electromyography (EMG) activity may be recorded. Applying appropriate SCS parameters to provide a therapeutically effective amount of electrical stimulation is expected to increase the maximum torque and the EMG activity.

This example aims to measure immediate, end-of-study, and long-term effects of SCS on SMA including various clinical outcomes. First, the example measures the immediate effects of SCS on SMA by turning on and off stimulation in the same session and measuring effects of SCS that are only present during stimulation and disappear when stimulation is turned off. As used herein, “immediate” refers to effects measured during the four-week study and relates to the usage of SCS. Second, the example measures the end-of-study effects of SCS on SMA by comparing performance of the patients without stimulation over different sessions. As used herein, “end-of-study” refers to effects measured within about 1 week before or after the end of the four-week study, around the time that the SCS electrodes are removed. Third, the example measures the long-term effects of SCS on SMA by following up with the patients after the end of the study, after the stimulation has been removed for several weeks. In particular, as used herein, “long-term” refers to effects measured 50+ days after the electrodes are removed and relates to changes in motor ability over time without the continuous application of SCS. Both immediate and long-term effects of SCS on the SMA patients were observed. In contrast to what would be expected in SMA-affected patients, the magnitude of the immediate effects was similar to those observed in patients with stroke and spinal cord injury. For subjects with spinal cord injury and/or stroke, the immediate effects outperform the long-term effects of SCS. Conversely, for subjects with SMA, the long-term effects outperform the immediate effects of SCS. Moreover, the magnitude of long-term effects observed over only 4 weeks of trial was unexpected for patients with non-functioning MNs, who experienced improvements in muscle strength beyond what a normal SMA-affected patient is expected to achieve. These improvements in muscle strength are too high to be attributed to exercise only. For example, left hip flexion of SMA01 more than doubled, but the patient did not perform any hip strength training other than walking during the study. SMA01's exercise level was unchanged from his exercise level before the study. These changes were reflected in improvements in the clinical outcome tests such as the manual muscle test, the Hammersmith Functional Motor Scale Expanded (HFMSE), the Revised Hammersmith Scale (RHS), and the 6-minute walk test for SMA01 and SMA02. Moreover, the data does not indicate that a plateau has been reached, indicating that longer use of SCS may lead to even greater improvement. Because SMA02 is older and has more severe motor impairment than SMA01, his improvements in muscle strength were smaller than but consistent with SMA01's results. However, end-of-study and immediate improvements in gait were more prominent in SMA02 than in SMA01, who had only mild gait deficits.

Further, for SMA01, over the course of 4 weeks, SCS therapy increases leg joint torques (up to +180%) and MN firing rates, indicating that SCS was improving MN function while reporting no side effects. These results indicate that SCS can potentially increase the quality of life of severe and mild SMA patients by improving muscle strength and gait. Such improvements of this magnitude within such a short time frame observed in patients with SMA are unexpected. The observed improvement in muscle strength of this magnitude, and in such a short time frame, may be indicative of a disease-modifying effect. Without being bound to any particular theory, the improved strength may be driven by rescued MN function in response to increase afferent inputs driven by SCS targeting the hip flexors and knee extensors afferents in the patients. Spinal reflexes and single MN firing rates obtained with HD-EMG recordings were analyzed to corroborate the conclusion. Furthermore, fMRI analyses show an increase in activity at the spinal circuits pre-implant versus post-explant. This increase may indicate that the stimulation rescued MN function by artificially increasing the afferent inputs.

### Initial Configuration and Optimization

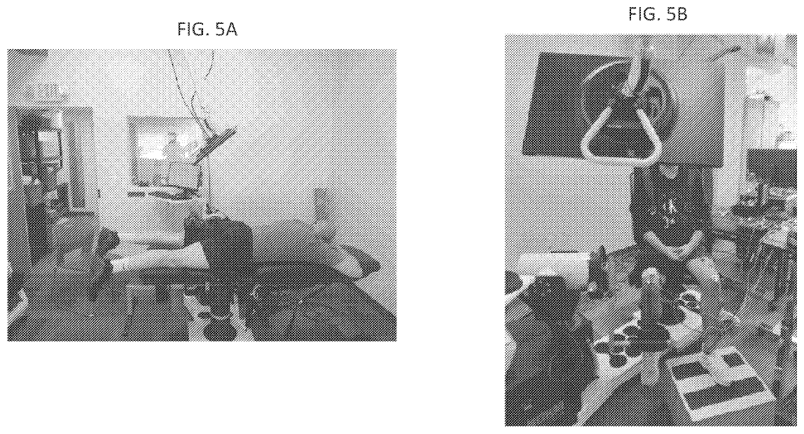

Before implantation of electrodes, the initial performance of the patients can be measured. The HUMAC® Norm system can be used for these measurements. With this system, the patients can be placed in different positions to evaluate the maximum torque of different joints. FIGS. 5A and 5B illustrate an isokinetic machine (e.g., a HUMAC® Norm isokinetic machine) configured to test hip flexion and knee extension on human patients, respectively. In particular, the maximum torques produced by the patients during the extension and/or flexion at knee, hip, and ankle can be measured.

Bilateral linear SCS leads were implanted in the epidural space from T11 to L1 vertebrae. To target the right leg muscles, in one example, the spinal cord was stimulated using bipolar stimulation of the two most rostral contacts of the right lead. The SCS parameters in this example were 2 mA, 40 Hz, and 400 μs pulse width. To target the left leg muscles, in one example, tripolar stimulation was used. The SCS parameters in this example were 3.7 mA, 40 Hz, and 400 μs pulse width.

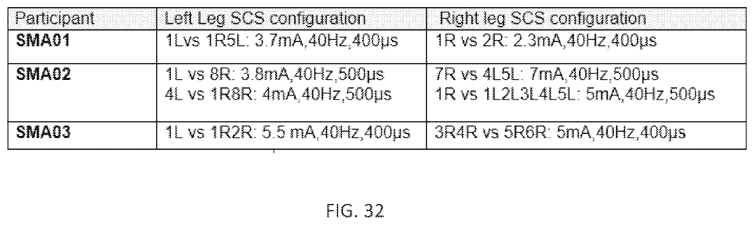

FIG. 32 illustrates a table of optimal current values and lead configurations for SMA01, SMA02, and SMA03. Electrode naming is coded from 1 to 8 and with a letter indicating whether it is the left or right electrode. For example, 1L indicates the most rostral contact of the left array. 7R indicates the second-last caudal contact of the right lead. When indicating configuration, the first contact is the cathode, and contacts after “vs” are anodes. For example, 1L vs 1R 5L means that 1L is selected as cathode, while 1R and 5L are both selected as anodes.

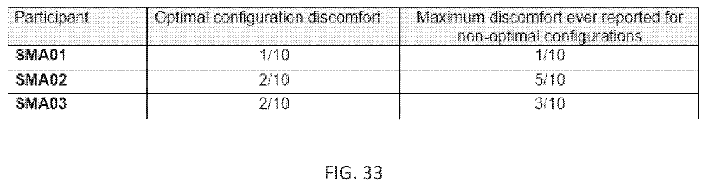

The administration of therapeutically effective SCS treatment through the implanted leads did not cause significant discomfort or pain according to the subjects. SCS produces tingling sensations and other types of sensory phenomena, but the stimulation intensities required to improve motor function remained within a range of non-painful sensations. As shown in FIG. 33, SMA01, SMA02, and SMA03 were asked to provide a score from 1 to 10 (where a greater number indicates a greater amount of discomfort) for each stimulation configuration. In the optimal configuration, the maximum discomfort rating provided by any of the subjects was 2 out of 10. The maximum discomfort provided in a suboptimal configuration was 5 out of 10.

SCS can be administered to target muscles affected by SMA. In the example, it is found that SMA01 had significant deficits in knee extensors and hip flexors, consistently with his Type 3 diagnosis. Further, SMA01 had slightly more deficits in the left leg. Accordingly, the stimulator could be programmed to selectively target these muscles. In the example, all experiments were performed over the course of 4 weeks and a total of 19 sessions. The experiments were performed daily for 5 days per week until the date on which the electrodes are explanted. Each session lasted 4 hours with approximately 2 to 3 hours of time-on-task, with an estimated dose of stimulation active for 2 hours per day during these sessions.

The location for administering SCS could be optimized and fine-tuned by stimulating various contact points, measuring electrical activity produced by the muscles (e.g., agonist and antagonist muscles of each joint), and evaluating the measurements. Maximum torque and the EMG activity are then measured to determine a therapeutically effective amount of electrical stimulation using appropriate SCS parameters. In the example, during surgery, the positions of the electrodes were determined by stimulating each contact and recording EMG signals of muscles ranging from the trunk down to the ankles. Stimulations were repeated until a location is identified where the electrodes activate the hip muscles with the most rostral contacts and the calf muscles with the most caudal contacts.

These implants can be used to configure electrical spinal cord stimulation procedures that allow control over the degree of extension and flexion of each leg during locomotion. This protocol uses real-time processing of gait kinematics and locomotor performance and can be configured within a few days. Once configured, stimulation bursts are delivered over specific spinal cord locations with precise timing that reproduces the natural spatiotemporal activation of MNs during locomotion. These protocols can also be easily adapted for the safe implantation of systems in the vicinity of the spinal cord and to conduct experiments involving real-time movement feedback and closed-loop controllers, as discussed below.

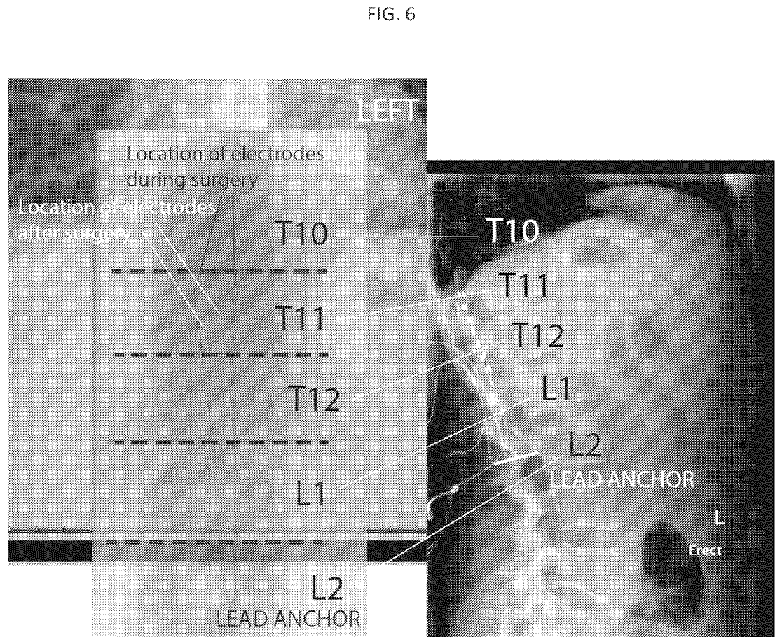

The placement of the electrodes during surgery can be adjusted to account for movement of the electrodes post-surgery. Such post-surgery movement may be variable and difficult to avoid with non-permanent implants. For example, the electrodes may move caudally and shift medially. FIG. 6 illustrates a comparison between the locations of the electrodes during the surgery (in black) and the locations after the surgery (in white) in a subject. In this particular example, the change of locations did not prevent the stimulation to the correct group of muscles. Based on predicted post-surgery movement of the electrodes, the electrodes can be implanted at adjusted locations during surgery such that, after the surgery, the electrodes would move to the predefined optimal locations. Additional details on the equipment and surgical procedures that can be used to acquire chronic EMG recordings from leg muscles and to implant targeted spinal cord stimulation systems can be found in Capogrosso et al., 2018. Nature Protocols 13. 2031-2061, which is incorporated by reference herein.

### Observed Immediate Effects

The immediate effects of SCS were measured in terms of maximum voluntary contraction, hip flexion during locomotion, balance, sit-to-stand transitions, maximum speed, and overground walking gait. Each measurement is discussed below. It should be appreciated that different metrics can be used depending on the targeted muscle(s) and conditions of a patient.

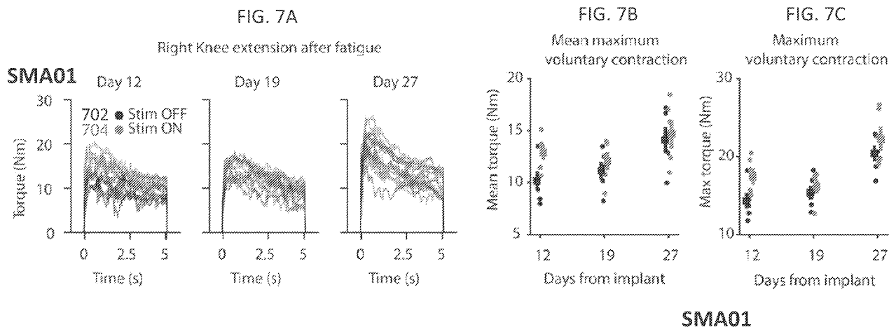

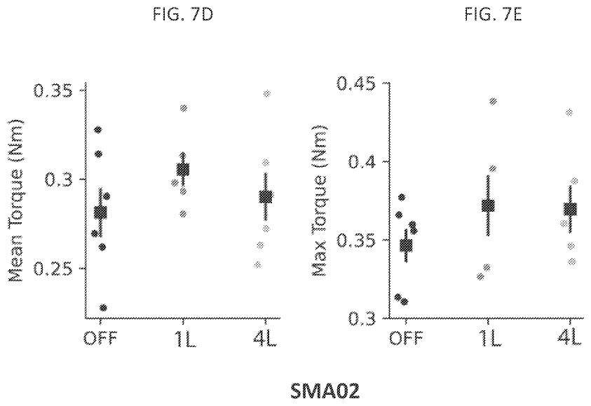

Maximum Voluntary Contraction: To evaluate maximum voluntary contraction, SMA01 was asked to produce his maximum voluntary isometric contraction (MVC) during knee extension for 5 seconds with SCS and without SCS. After several repetitions of knee extensions, SMA01 would show clear signs of fatigue. At that time, the immediate impact of SCS on the patient's maximum voluntary contraction (MVC) during fatigued knee extension was tested. FIGS. 7A-7C illustrate the various torque measurements over time. FIG. 7A illustrates single traces of torque produced by SMA01 with stimulation (see element 704) and without stimulation (see element 702) during maximum voluntary contraction repetitions. In FIG. 7B, each dot corresponds to the mean torque produced during one single trial with and without stimulation. In FIG. 7C, each dot corresponds to the maximum torque produced during one single trial with and without stimulation. In FIGS. 7B and 7C, the square markers represent the mean across repetitions, and the error bars correspond to the standard error of the mean. As shown, the MVC during SCS stimulation was significantly higher on days 12 and 27.

Likewise, SMA02 was asked to produce his MVC during knee extension for 2 seconds with SCS and without SCS. Because SMA02 is only able to produce very small forces, increases in strength are not statistically significant between stimulation being on or off, or between different stimulation parameters, as shown in FIGS. 7D and 7E. FIGS. 7D and 7E depict torque measurements obtained during knee extension in SMA02 at baseline and with two different stimulation contacts, 1L and 4L. As shown, mean torque was higher for 1L than baseline, but not for contact 4L. Overall peak torque was higher for both 1L and 4L. The figures show that contact configuration affects torque output.

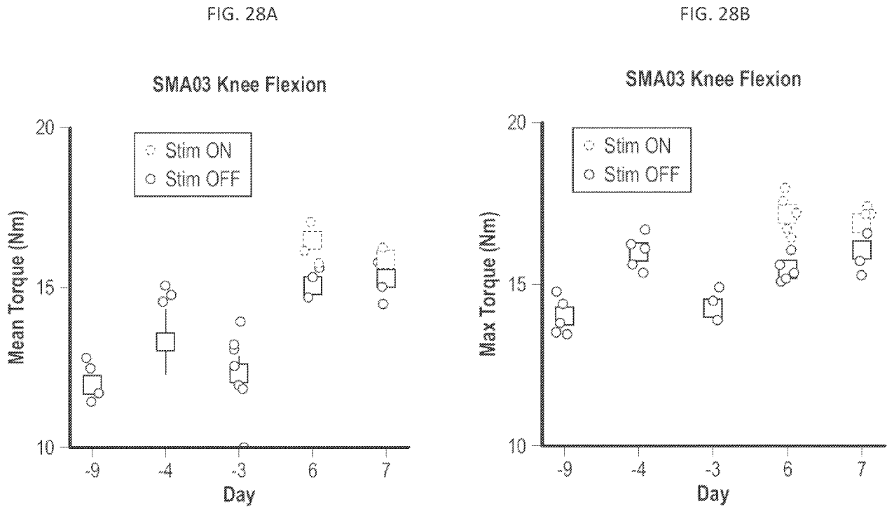

To obtain more data on the role of SCS in immediately increasing muscle strength (as measured by torque produced by MVC), SMA03 was asked to produce his MVC during knee flexion with SCS and without SCS. FIGS. 28A-28B illustrate the various torque measurements across trials on different days. Each dot corresponds to the mean/maximum torque produced during one single trial, the square markers represent the mean across trials on the same day, and the error bars correspond to the standard error of the mean. Prior to implantation of the contacts (e.g., on days −9, −4, and −3), baseline values for SMA03's knee flexion abilities without SCS are measured. After implantation (e.g., on days 6 and 7), the knee flexion torque produced by SMA03 with stimulation and without stimulation were measured. FIG. 28A illustrates the mean torque produced during trials with and without stimulation. When stimulation was applied, the mean torques produced were greater than when stimulation is off. Likewise, FIG. 28B, which illustrates the maximum torque produced during trials with and without stimulation, depicts that maximum torques are greater with stimulation on than with stimulation off. In both figures, the torques with stimulation are greater than the baseline torques before implantation and the torques without stimulation. These figures indicate that applying stimulation via SCS immediately increased muscle strength beyond a patient's normal ability without stimulation.

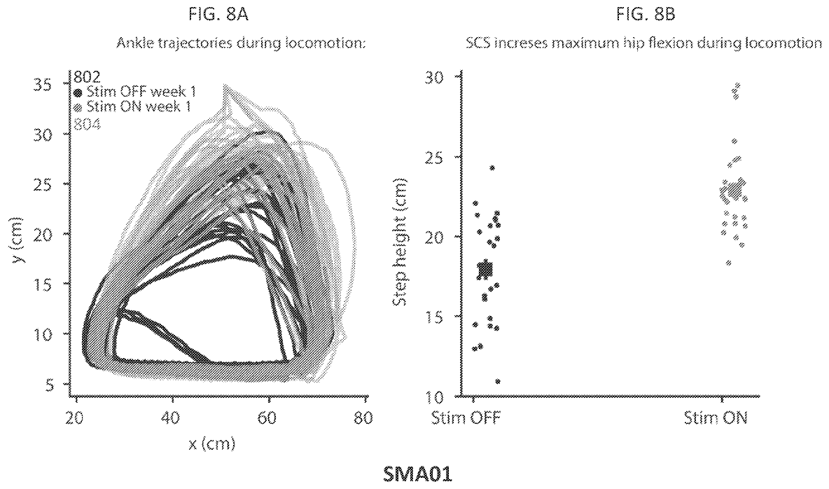

Hip Flexion During Locomotion: SMA01 was asked to produce maximum hip flexion by raising his knees as high as possible while his body weight was supported by a treadmill. While the patient was walking, periods of stimulation were alternated on and off every 20 seconds. SMA01 was given no visual feedback of his legs during the task. To measure changes in step height, tracking markers were placed on the ankle, toe and metatarsal on each foot. A machine-learning model (e.g., a deep neural network) could be trained to track the markers. FIGS. 8A-B illustrate that spinal cord stimulation increases maximum hip flexion during locomotion. Specifically, FIG. 8A illustrates traces of an ankle marker during locomotion without stimulation (see element 802) and with stimulation (see element 804). In FIG. 8B, the dots represent the height of each step computed as the difference between the smaller and the higher values of each trace in FIG. 8A. The squares markers represent the mean across steps, and the error bars correspond to the standard error of the mean. As shown in FIGS. 8A-8B, SCS provided an immediate increase in SMA01's maximum hip flexion during locomotion, which translates to a higher step height.

Balance: SMA01 was asked to walk heel-to-toe while staring straight ahead on a beam that becomes narrower with distance, while receiving no visual feedback of his feet. In the first session, SMA01 was worse with stimulation on and consistently traverses less distance. The patient reported that the stimulation perturbed his balance because the precise control of his legs became more difficult.

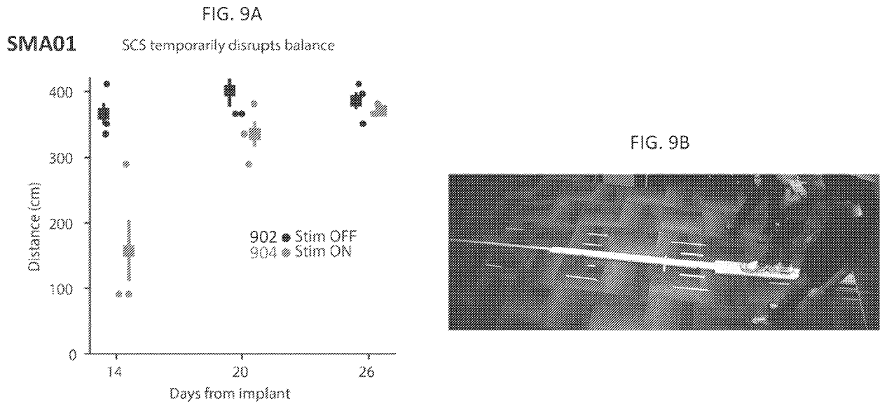

However, when SMA01's balance was re-tested during the second session (a week after the first session) and the third sessions (a week after the second session), it was found that the patient learned to control the stimulation and improved his balance such that stimulation on and off would produce similar distances. FIGS. 9A-9B illustrate that spinal cord stimulation on the patient only temporarily disrupted balance. In FIG. 9A, each dot is the walked distance for SMA01 in the narrowing beam test without stimulation (see element 902) and with stimulation (see element 904). The squares represent the mean and error bars represent the standard error of the mean across repetitions. FIG. 9B is a picture of SMA01 walking on the narrowing bean.

Sit-to-Stand Transitions: SMA01 was asked to stand from a position where one knee is on the ground and the opposite foot is planted on the ground. Different knee heights were tested until the maximum height where the patient was unable to stand is identified. The stimulation was then applied. SMA01 was able to consistently reach a standing position when the right knee was planted on the ground.

The patient was also asked to stand from a sitting position on a box measuring 46 cm from the ground. Without stimulation, SMA01 was able to perform compensated standing while his legs are exaggeratedly spread out. The patient was then instructed to continue to attempt to stand bringing his feet closer together until a separation distance between his feet was reached where he was no longer able to stand. The stimulation was then applied to the patient with his feet in the same position, i.e., separated at the distance where he was unable to stand, and SMA01 was able to consistently stand from such position.

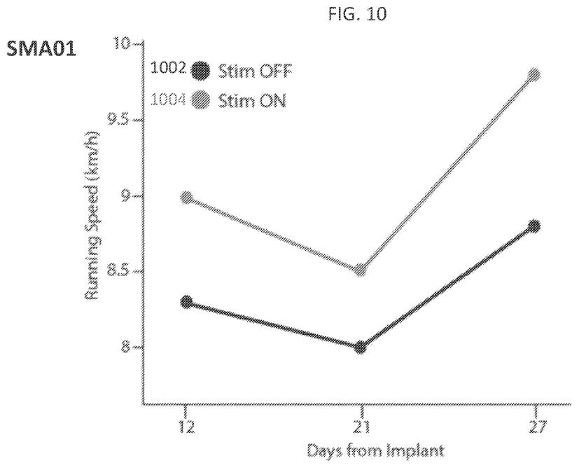

Maximum Speed: SMA01 was asked to run on the treadmill, and the velocity was progressively increased every 30 seconds until he reported that his maximum velocity was reached. FIG. 10 illustrates that SCS (see element 1004) robustly increased the patient's maximum velocity relative to when SCS was not applied (see element 1002).

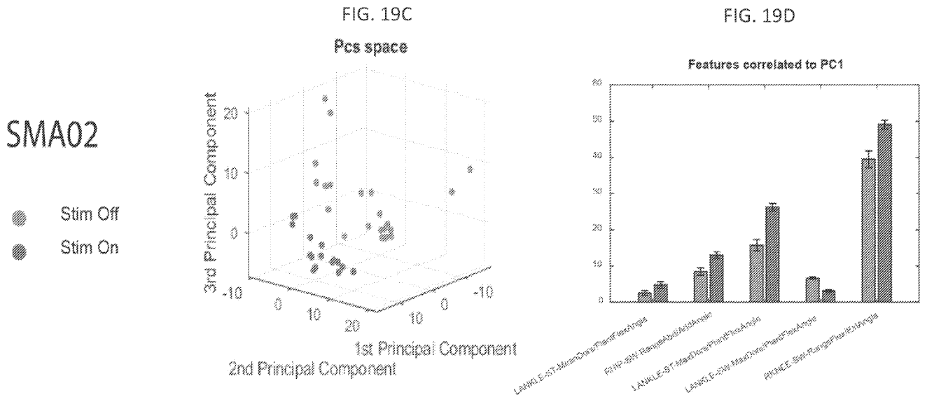

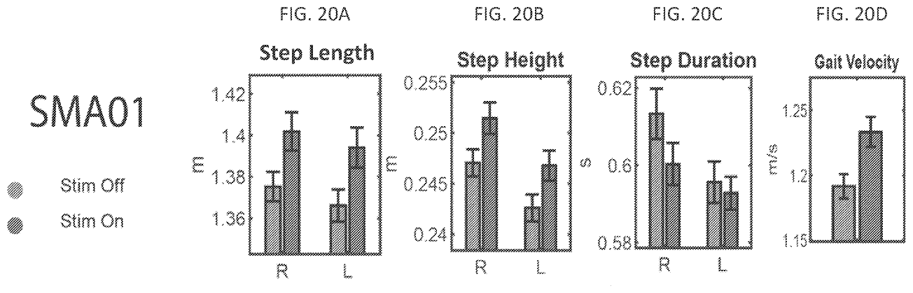

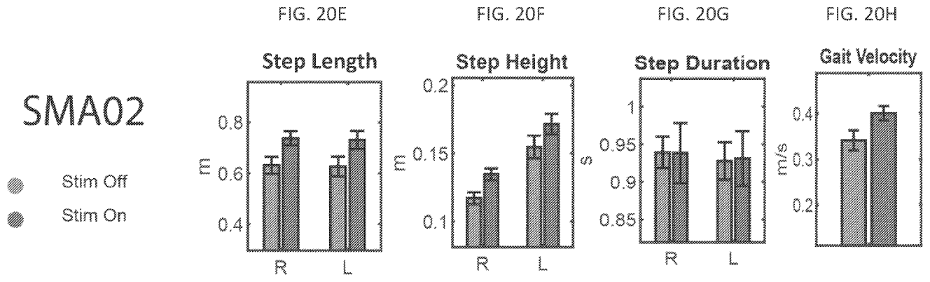

Overground Walking Gait: SMA01 and SMA02 were asked to walk back at a self-selected speed with and without stimulation. The Vicon® system was used to record the position and orientation of reflective markers attached to specific anatomical landmarks, from which the joint angles, velocities and accelerations were then reconstructed. In order to evaluate the kinematic variables that explain most of the variance between trials with and without SCS, the principal component analysis (PCA) was performed. For SMA01, the variables that may explain most of the variance were related to the velocity of the knee flexion/extension during the swing phase, indicating that with stimulation the knee flexion movement is faster, as shown in FIG. 19B. The stimulation was found to increase the gait velocity and decrease the step duration, as shown in FIGS. 20C and 20D.

The PCA analysis of SMA01 shows that the stimulation reduces the knee rotation during the swing phase, indicating a reduction in compensatory strategies during gait. The knee was less externally rotated during the stimulation ON trails, which may indicate a reduction in compensatory strategies during gait. FIGS. 19A and 19B illustrate the PCA results for SMA01. FIG. 19A illustrates a Principal Component (PC) space composed for the first three PCs. The separation between trials with stimulation on versus trials with stimulation off is clear along the direction of the third PC. As a whole, the trials with stimulation on have lower third PC values than the trials with PC off. FIG. 19B illustrates the gait features that are more strongly correlated with the third PC. FIGS. 19C and 19D illustrate similar PCA results for SMA02. FIGS. 20A-20D illustrate the short-term improvement of several gait quality variables for SMA01. These figures show that, for both his right and left sides, step length increases (FIG. 20A), his step height increases (FIG. 20B), and his step duration decreases (FIG. 20C) with stimulation. FIG. 20D illustrates that SMA01's step duration improved, i.e., decreased, with stimulation. FIGS. 20E-H illustrate short-term improvement of several gait quality variables for SMA02 similar to those of SMA01 when stimulation was on.

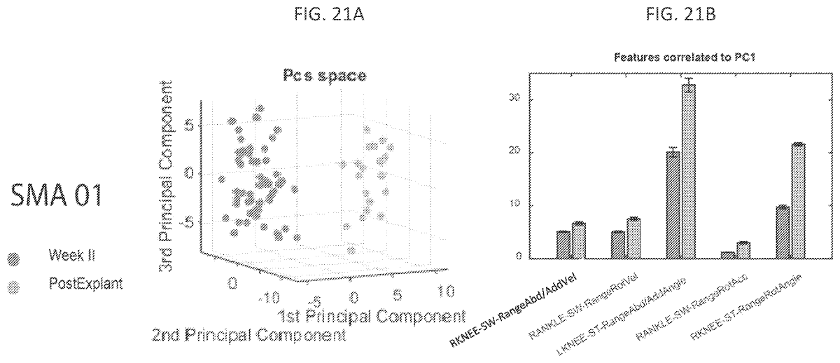

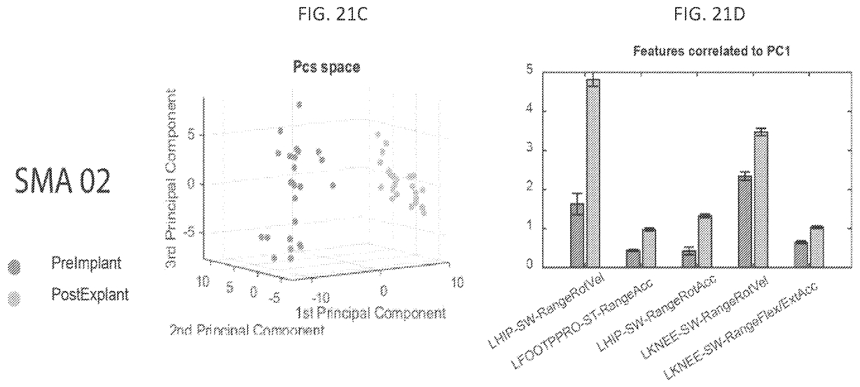

The PCA analysis of SMA02 shows that the stimulation improved different gait patterns:
1) The stimulation increased the range of hip abduction/adduction and knee flexion/extension (as shown in FIG. 19D), as well as the maximum hip flexion angle (as shown in FIGS. 21C and 21D). As a result, SMA02 was able to make longer and higher steps than without stimulation (as shown in FIGS. 20E and 20F).
2) The stimulation also increased the maximum ankle dorsiflexion angle during the stance phase (as shown in FIG. 19D), improving the foot clearance and toe lifting (as shown in FIGS. 21C and 21D).

Additionally, the methods of the present disclosure were found to reduce the compensatory strategies of people with SMA. For instance, people with SMA tend to compensate for their weak quadriceps during the swing phase by plantar flexing their foot to increase hip flexion. Both SMA01 and SMA02 reduced this compensatory strategy with stimulation: they exhibited reduced plantarflexion and increased hip flexion. Thus, the results indicate that SCS has the potential to produce changes in kinematics dynamics that immediately alleviate gait deficits during overground walking.

### Observed End-of-Study Effects

In this example, the end-of-study effects of SCS were measured in terms of hip flexion during locomotion, maximum voluntary contraction, overground walking gait, and functional magnetic resonance imaging of the spinal cord. The measurements were taken shortly before or after (i.e., within 1 week of) removal of the electrodes in the patient and illustrate the effects of SCS at the end of the four-week experiment. Each measurement is discussed below. It should be appreciated that different metrics can be used depending on the targeted muscle(s) and conditions of a patient.

No serious adverse events (e.g., falls) related to SCS were observed during the four-week experiment. Injury during falls is avoided due to risk mitigation strategies in place for the experiment, such as straps, harnesses, and/or presence of a physical therapist during physical activity.

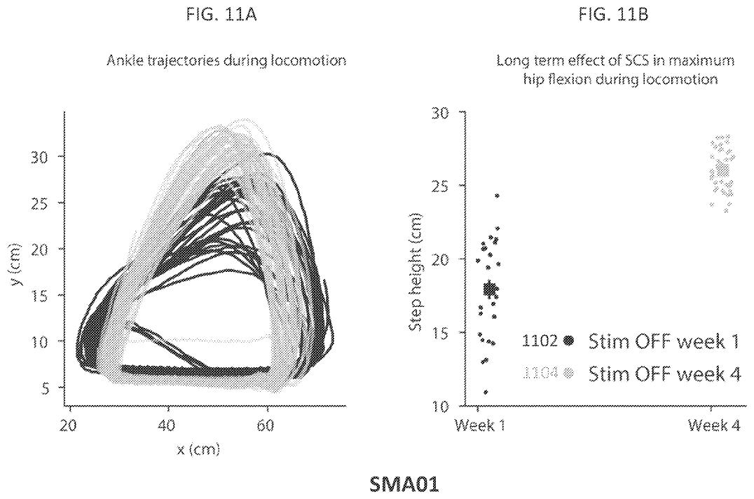

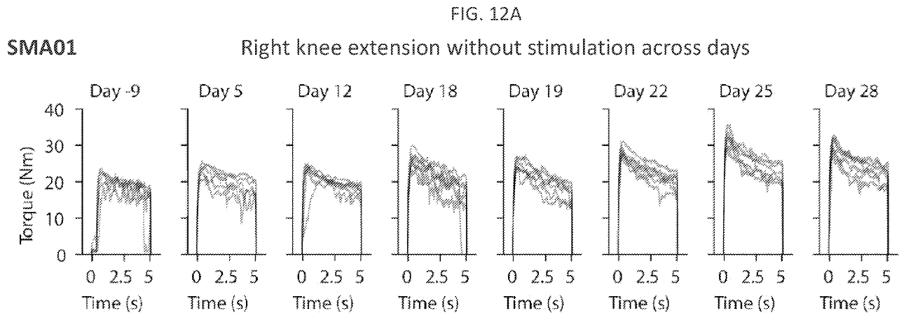

Hip Flexion During Locomotion: SMA01 was asked to walk on the treadmill while raising his knees as high as possible in week 4. FIGS. 11A-11B illustrate an end-of-study effect of SCS in hip flexion over a four-week experiment. FIG. 11A illustrates traces of the ankle marker during locomotion, comparing traces from week 1 without stimulation (see element 1102) to traces from week 4 without stimulation (see element 1104). In FIG. 11B, the dots are the height of each step computed as the difference between the smaller and the higher values of each trace in FIG. 11A. The square is the mean and standard error of the mean across steps. By comparing week 1 and week 4, a noticeable increase is observed in the SMA01's maximum hip flexion during locomotion even without stimulation, indicating an end-of-study effect of the SCS intervention. Without being bound to any particular theory, application of the electrical stimulus to the patient over time may lead to ion channel remodeling on the MN membrane and a persistent increase in the firing rate probability of spinal MNs which may improve motor function, even in the absence of stimulation.

Maximum Voluntary Contraction: At the beginning of each session with the isokinetic machine, the MVC of SMA01 and SMA02 were measured as torque in Newton-meters (Nm) during knee extension and/or hip flexion without stimulation. The MVC of the patient during knee extension was about 20 Nm, while the MVC of a healthy young adult is above 100 Nm. SMA01 was asked to produce his maximum torque in these joints for 6 repetitions of 5 seconds each. To prevent early fatigue, SMA02 was asked to produce his maximum torque in these joints for 6 repetitions of 2 seconds each. The knee extension was tested twice per week and hip flexion is tested pre-implant, in week 1, in week 3 and post-explant. In general, significant increases were observed in MVC for all movements starting from week 2.

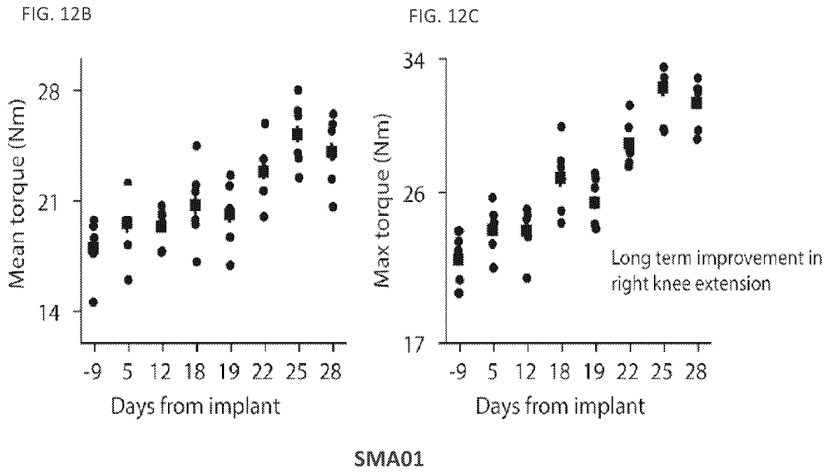

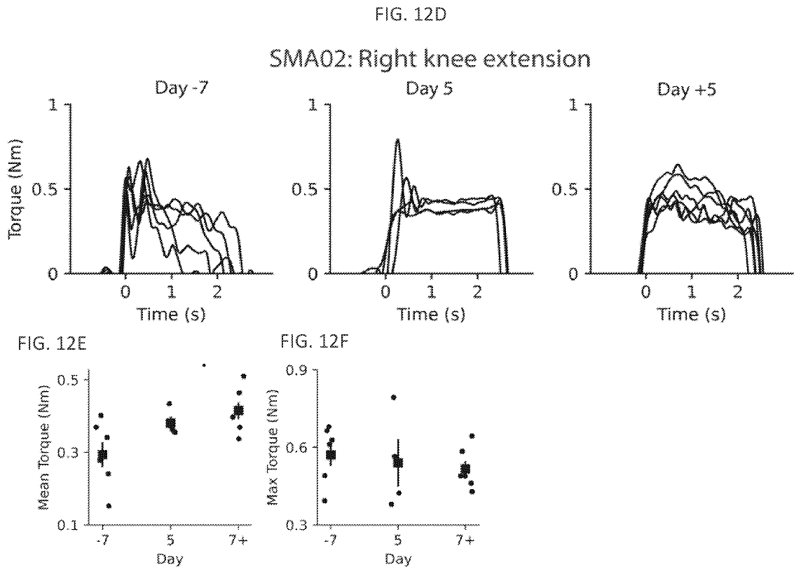

In terms of right knee extension, starting from day 18 to the end of the experiment, a consistent increase in maximum and mean torque was observed when SCS is turned off. The final increase from pre-implantation to the end of study was +43.5%. FIGS. 12A-12C illustrate end-of-study improvement in right knee extension of SMA01. FIG. 12A illustrates torque traces for the 6 repetitions in each session. In FIGS. 12B-12C, each dot is the mean torque for each 5-second repetition. The squares are the mean and the standard error of the mean of the mean torque across repetitions. As shown, the effect appears to be linear and there is no evidence of reaching a plateau by week 4. Starting from day 18 to the end of the experiment, SMA01 experienced a consistent increase in maximum and mean right knee extension torque when SCS is turned off. The final increase from pre-implantation to the end of study was +43.5% for the SMA01.

The MVC of SMA02 during knee extension was below 1 Nm, while the MVC of SMA01 was above 20 Nm, and the MVC of a healthy young adult was above 100 Nm. Accordingly, the knee extension of SMA02 was more limited than that of SMA01. FIGS. 12D-12F illustrate end-of-study improvement in right knee extension for SMA02. The increase in mean torque from pre-implantation to the end of study was +33.3% for SMA02 (as shown in FIG. 12E). However, no noticeable difference in his peak torque was observed (as shown in FIG. 12F).

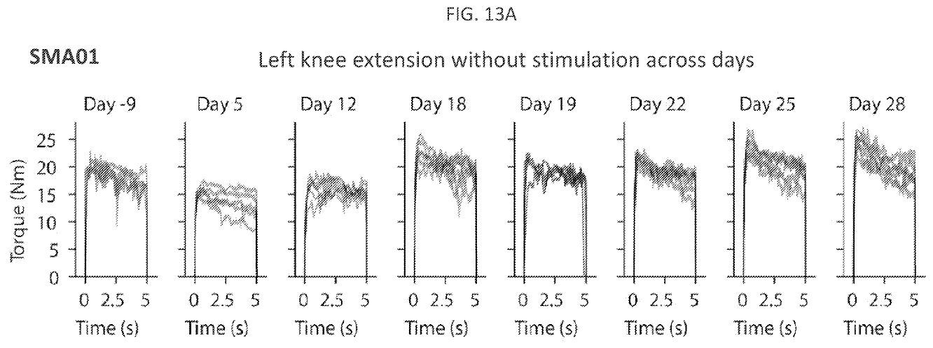

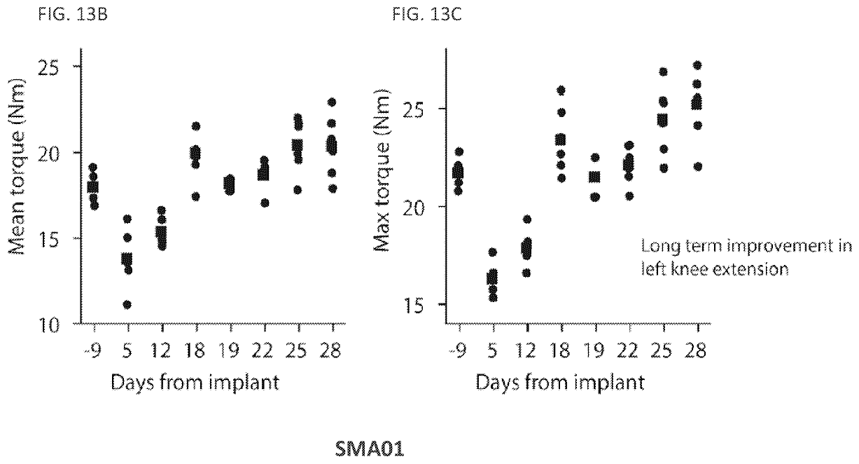

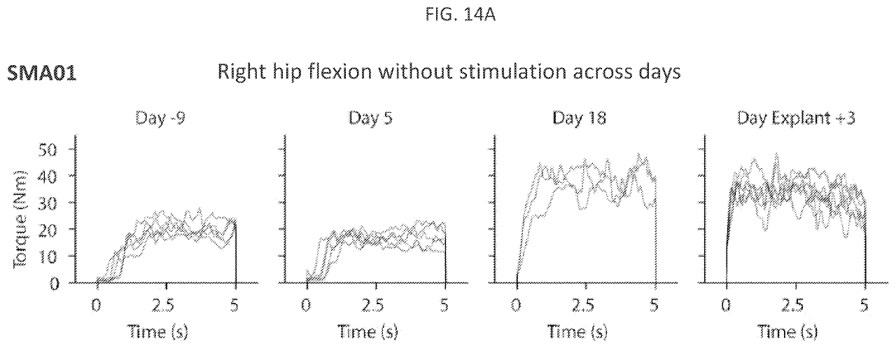

A consistent increase in left knee extension of the SMA01 was similarly observed from day 18 to the end of the four-week experiment, even though it was SMA01's more impaired leg. Left knee extension results during the four weeks of SCS experiments were largely consistent with the results for the right knee extension, in that no increase was observed before day 18 and sustained increase is then observed afterwards. The final increase from day 5 to end of study was +65.1%. FIGS. 13A-13C illustrate end-of-study improvement in left knee extension. FIG. 13A illustrates torque traces for the 6 repetitions in each session. In FIGS. 13B-13C, each dot is the mean torque for each 5-second repetition. The squares are the mean and the standard error of the mean of the mean torque across repetitions.

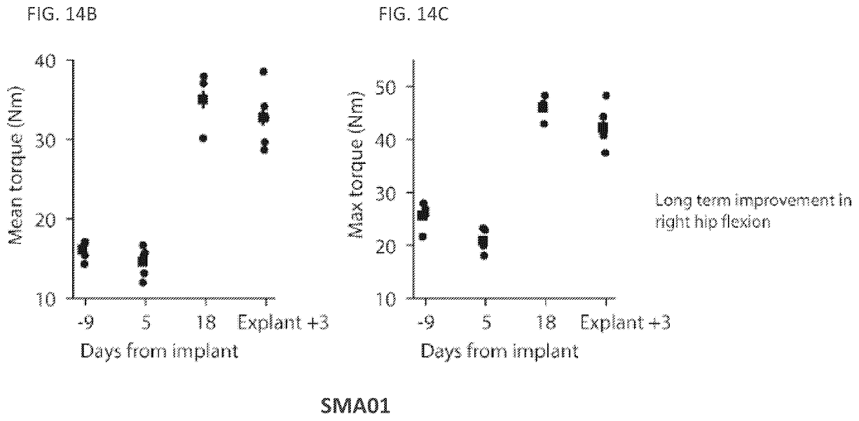

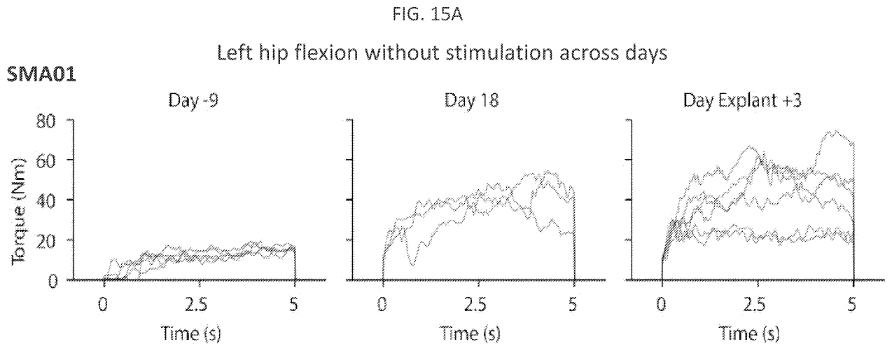

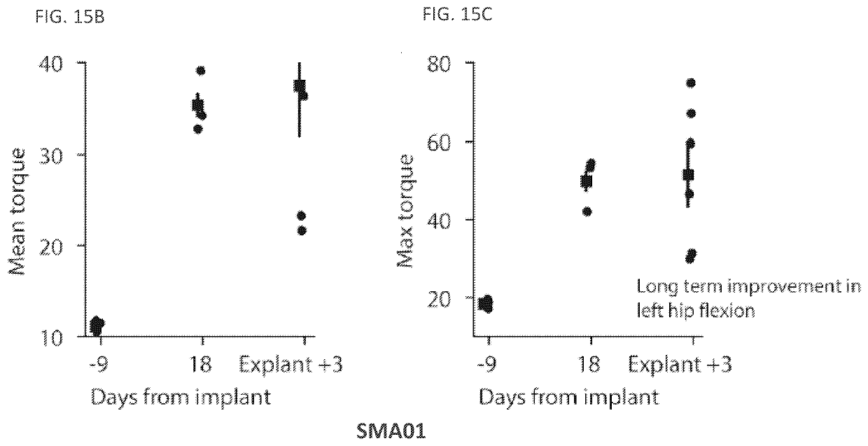

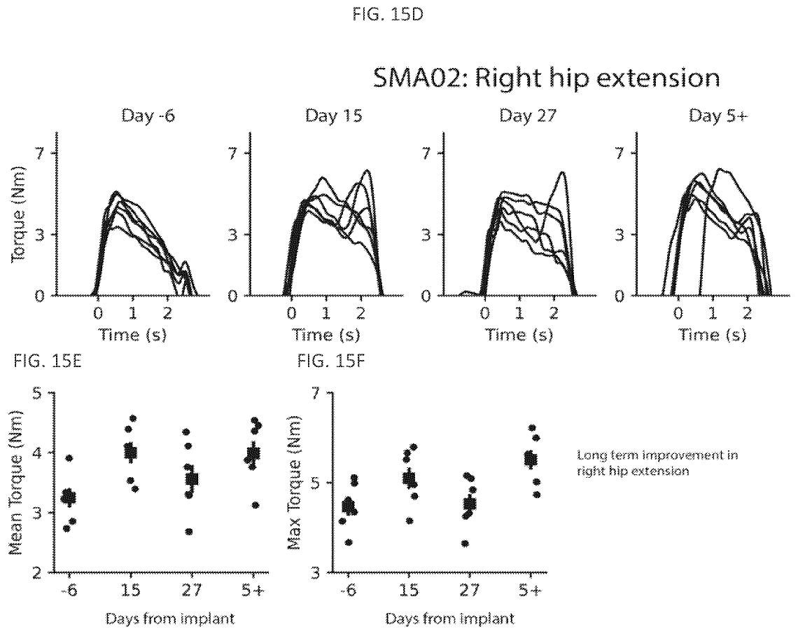

The timing of improvements in right and left hip flexion of SMA01 was consistent with the results in knee extension, with an increase in both being observed from day 18 to the end of the four-week experiment for hip flexion. The final increase from pre-implant to post-explant was +65.1% on the right and +179.7% on the left for SMA01. FIGS. 14A-14C illustrate end-of-study improvement in right hip flexion. FIG. 14A illustrates torque traces for the 6 repetitions in each session. In FIGS. 14B-14C, each dot is the mean torque for each 5-second repetition. The squares are the mean and the standard error of the mean of the mean torque across repetitions. FIGS. 15A-15C illustrate end-of-study improvement in left hip flexion for SMA01. FIG. 15A illustrates torque traces for the 6 repetitions in each session. In FIGS. 15B-15C, each dot is the mean torque for each 5-second repetition. The squares are the mean and the standard error of the mean of the mean torque across repetitions.

Right hip extension for SMA02 increased from pre-implant to post-explant by +21.7%. FIGS. 15D-15F illustrate end-of-study improvement in right hip extension for SMA02. FIG. 15D illustrates torque traces for the 6 repetitions in each session. In FIGS. 15E-15F, each dot is the mean torque for each 5-second repetition. The squares are the mean and the standard error of the mean of the mean torque across repetitions.

As a result of these improvements in hip flexion and knee extension, the patient's overall range of motion was much higher after the end of the experiment than before. The patient's improvements in range of motion were most noticeable in hip flexion and whole body movement. Without being bound to any particular theory, the application of the electrical stimulus to sensory neurons may directly recruit mono- and poly-synaptic excitatory pathways in the spinal cord, which in turn increases the membrane potential and firing rate probability of spinal MNs innervating the body region of the patient with the motor impairment due to SMA. This recruitment of pathways may increase neuroplasticity, allowing the patient to improve motor function.

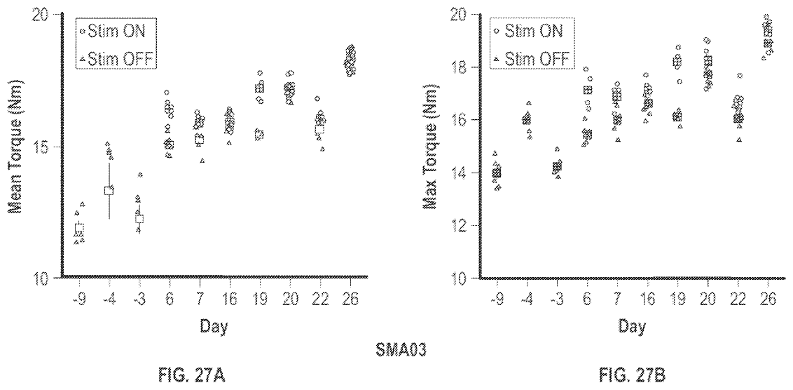

To obtain more data on the role of SCS in increasing muscle strength (as measured by torque produced by MVC) over the course of the study, SMA03 was asked to produce his MVC during knee flexion multiple times during the four weeks of the study. FIGS. 27A-27B, which expand upon the data sets from FIGS. 28A-28B, illustrate SMA03's various torque measurements across trials on different days. Each dot corresponds to the mean/maximum torque produced during one single trial, the square markers represent the mean across trials on the same day, and the error bars correspond to the standard error of the mean. Relative to the pre-implantation baseline values for SMA03's knee flexion abilities (e.g., the values on days −9, −4, and −3), the patient's MVC values show a noticeable upward trend throughout the course of the study (e.g., from days 6 through 26). Furthermore, during the study, knee flexion torques were measured with and without stimulation. When stimulation was applied (represented by the lighter dots for “Stim ON”), the torques produced are greater than when stimulation is off (represented by the darker dots for “Stim OFF”). FIG. 27A illustrates the mean torque produced increasing over the course of the study. Likewise, FIG. 27B illustrates the maximum torques produced increasing over the course of the study. In both figures, the torques measured at the end of the study (e.g., day 26), both with and without stimulation, are significantly greater than the baseline torques before implantation. These figures indicate that applying stimulation via SCS gradually increased muscle strength beyond a patient's normal ability over the course of the study. Even when SCS was not actively being applied at the end of the study. SMA03's muscle strength was greater than it was compared to the baseline values from the start of the study.

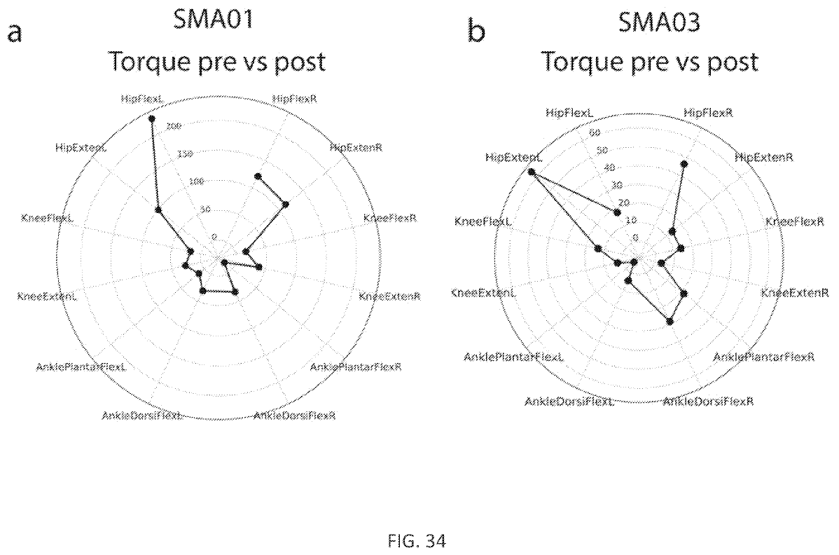

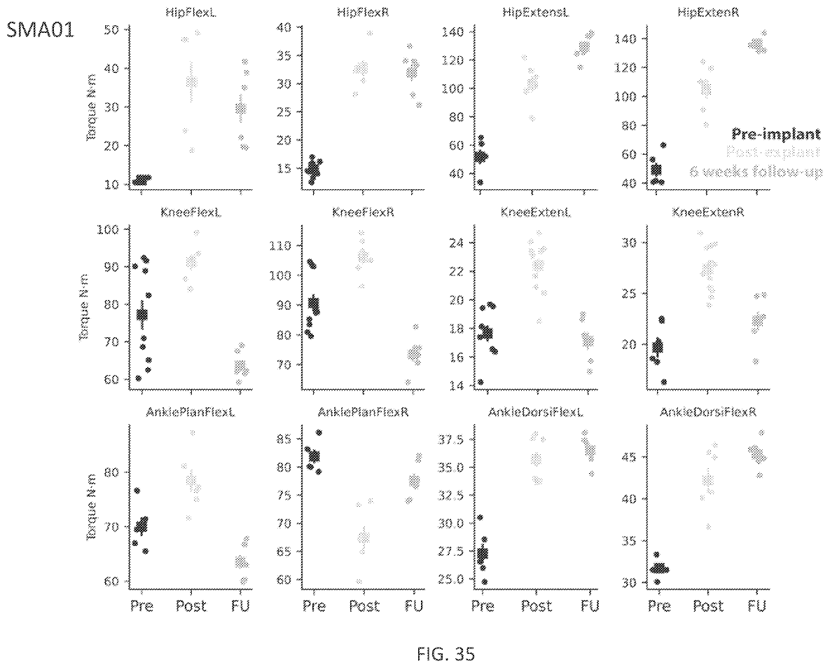

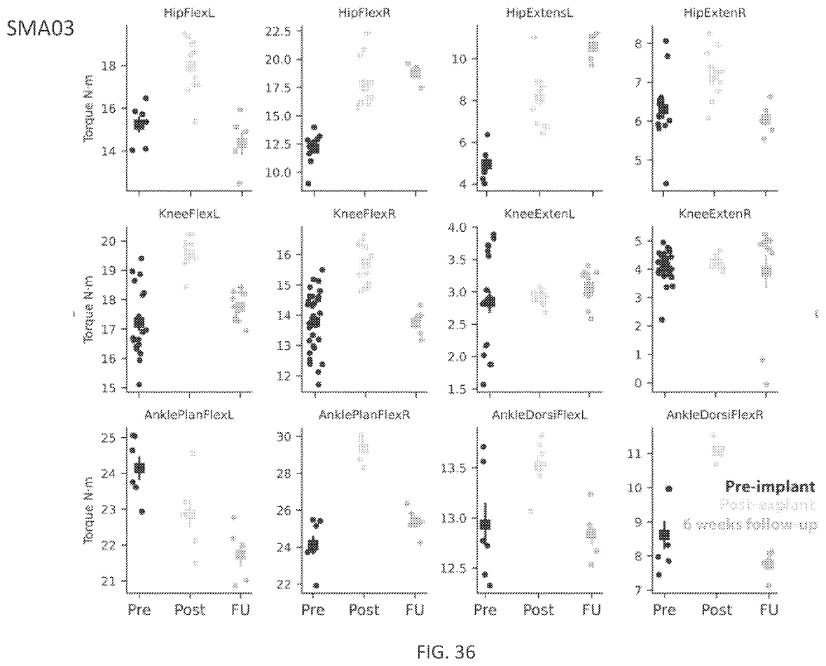

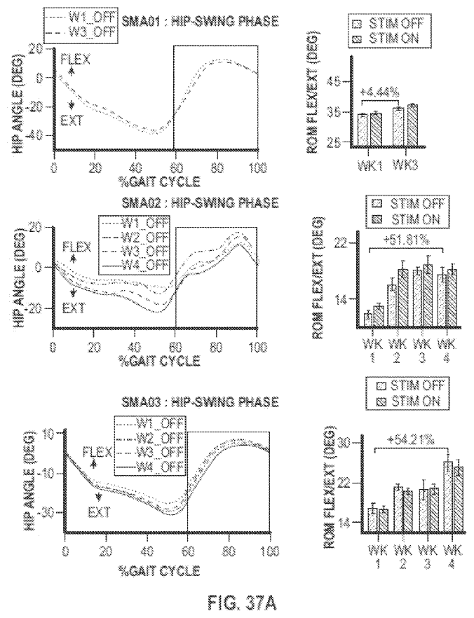

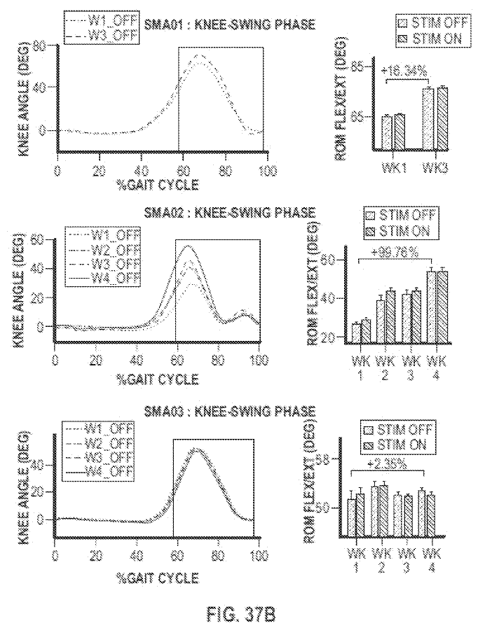

FIG. 34 illustrates, using web plots, the percentage increase in absolute torques at all joints over the course of the study for SMA01 and SMA03. (Raw data used to produce FIG. 2 can be found in the “Supplemental Data” section corresponding to FIGS. 35 and 36.) Clear improvements at the hip flexors and extensors were present in both participants when comparing pre-study and post-study results. At a follow-up appointment six weeks after the end of the study, the torques at the hip slightly increased, while the other torques return to pre-study levels. No SCS is administered during the collection of any of these results.

Despite large differences in disease severity, both subjects had similar results wherein substantial increases in isometric torques were observed mostly at the hip with changes as high as +200% increases. The size of these improvements in such a short period of time is suggestive of changes at the spinal MN level and specifically on efficiency of muscle recruitment. This is particularly true for hip muscles, which did not undergo weekly strength training. Hip torques are assessed only 3 times: pre study, mid-study and post-study, providing little strength training. Conversely, knee muscles were tested multiple times per week, thereby providing effective knee strength training. Therefore, the increase in hip function cannot be explained by strength training, yet it shows the highest improvements from SCS treatment. These findings correlate with the phenotypes of the Delta7 muse model described by the work of the Mentis lab (Mentis et al., 2011. Neuron 69 (3): 453-67), in which hip muscles are shown to be significantly more affected by SMA than ankle muscles. However, no other study, whether for treating SMA or for any other disease, has demonstrated such a magnitude of improvement (+200%) in leg muscle strength after four weeks. Strength training alone cannot account for these improvements, since it is not possible to “train” a participant to double the strength of a muscle in four weeks.

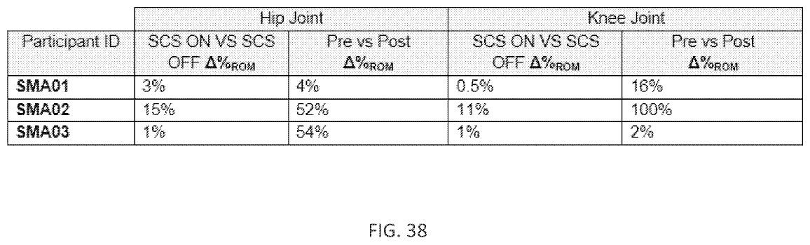

Range of Motion (ROM): ROM of the hip and of the knee during overground locomotion for SMA01, SMA02, and SMA03 is calculated following the study. The ROM is defined here as (max angle-min angle). This calculation refers specifically to angles during swing. FIGS. 37A-37B provide a gait cycle profile of joint angles and ROM for the hip and knee movements of SMA01, SMA02, and SMA03 at different weeks with SCS off. FIG. 38 presents a portion of FIGS. 37A-37B's data as a table of ROMs of the hip joint and knee joint for all three subjects, pre-study vs. post-study and SCS on vs. SCS off. As shown in the table, none of the participants improved more than 20% when comparing SCS on vs. SCS off. However, SMA02 and SMA03 improved more than 50% over time when comparing pre-study vs. post-study results. SMA01 experienced lower improvements, but this is due to the fact that SMA01 had the least advanced disease progression out of the subjects and falls within the normal biomechanical limits for ROM during locomotion.

Improvements at the hip were more prominent in SMA02 and SMA03. SMA02, in particular, also exhibited substantial improvements in knee ROM. This may be due to the fact that SMA02's severe disease progression and limited ROM allowed for the most room for improvement. Overall, for all subjects and all joints, ROM significantly improved in the pre-study vs. post-study comparison.

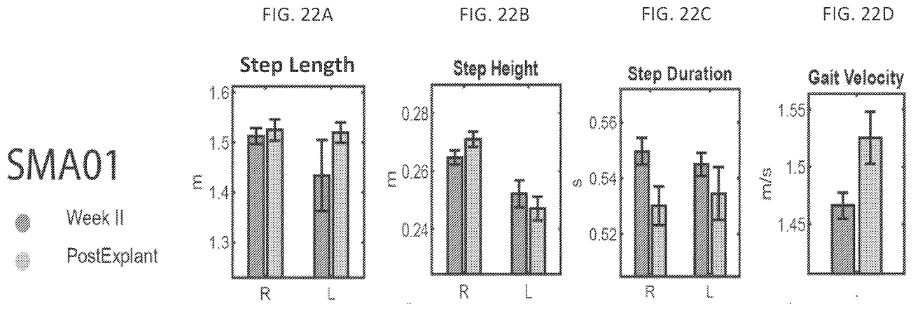

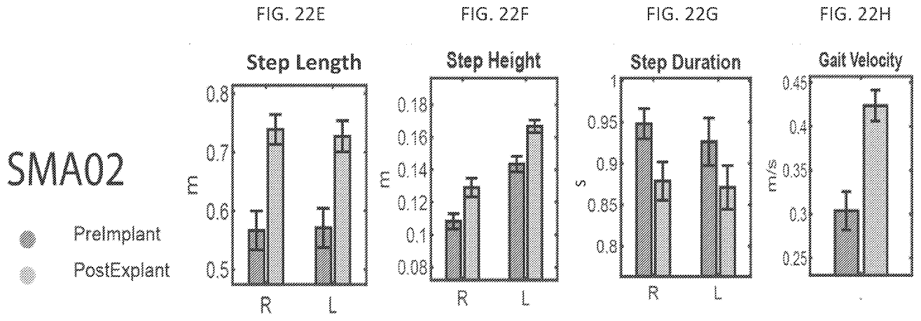

Overground Walking Gait: Changes in the gait patterns of SMA01 and SMA02 during the 6-minute walk test, wherein the subjects walk as fast as possible during the 6 minutes, were analyzed over a four week period. Following the same approach as the PCA experiment described in the “Observed Immediate Effects” section above, week 2 and post-explant data were compared for SMA01, and pre-implant and post-explant data were compared for SMA02. FIGS. 21A-21D illustrate the end-of-study effects on 6-minute walk test gait patterns across weeks for SMA01 and SMA02. FIGS. 21A and 21C illustrate a 3D space defined by the Principal Components (PC) for SMA01 and SMA02, respectively. FIGS. 21B and 21D illustrate histogram plots reporting the mean value of the variables that most contribute to the PC for SMA01 and SMA02, respectively. As shown in FIGS. 21A and 21C, for both subjects, the gait patterns of different weeks are clearly separable along the direction of highest variability (PC1), indicating that the stimulation produced changes in gait patterns. As shown in FIGS. 21B and 21D, for both subjects, the features that explain variance were primarily related to the velocity of the joints. For this reason, temporal and spatial gait variables that can be indicative of faster, more efficient gait were analyzed. FIGS. 22A-22D illustrate the end-of-study improvement of several gait quality variables for SMA01. These figures show that, for both the right and left sides of SMA01, step length increases (FIG. 22A), step height increases (FIG. 22B), and step duration decreases (FIG. 22C) over the duration of the study. FIG. 22D illustrates that SMA01's step duration improved, i.e., decreased, over the duration of the study. FIGS. 22E-22H illustrate end-of-study improvement of several gait quality variables for SMA02 similar to those of SMA01. The results of both subjects as shown in FIGS. 22A-22H consistently indicate that, as an end-of-study effect of the stimulation, the step length, step height, and gait velocity of each subject increased, while step duration decreased. Together, these results indicate that, over a period of only 4 weeks, the stimulation intervention described herein was able to alleviate gait deficiencies in people living with SMA.

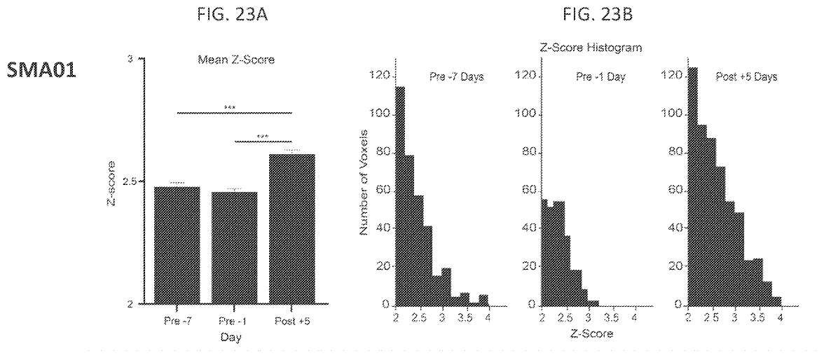

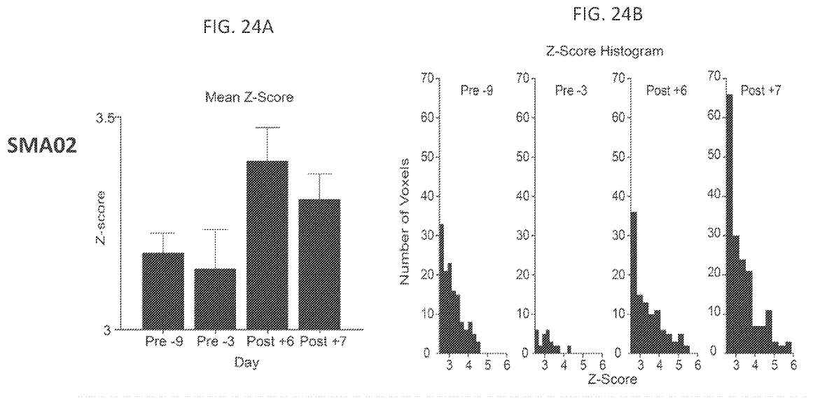

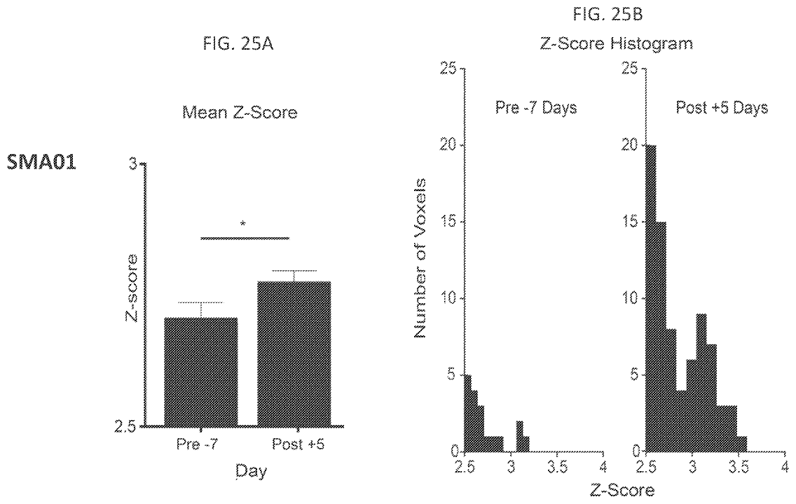

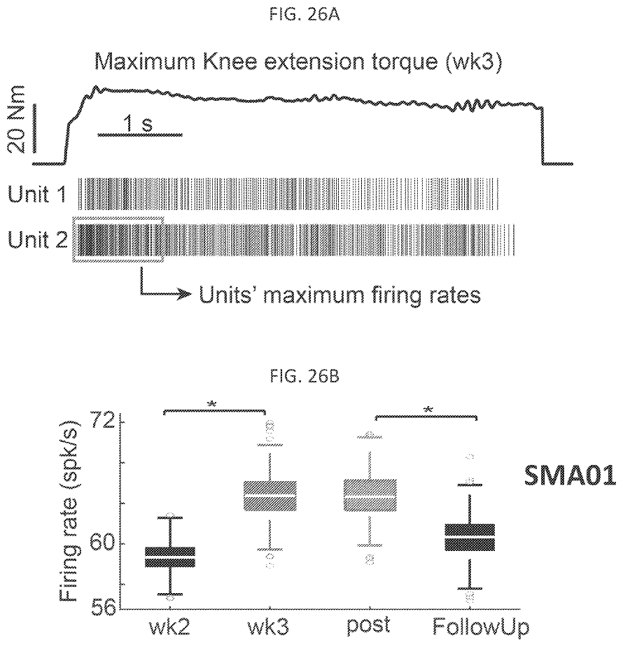

Functional Magnetic Resonance Imaging (fMRI) of the Spinal Cord: fMRI of the spinal cord was performed to assess whether functional reorganization of lumbar spinal activity occurs from end-of-study use of SCS. Without being bound by any particular theory, end-of-study improvements from SCS can revert the maladaptive changes in MNs ion-channels, improving MN dysfunction, and thus can be reflected in the hemodynamic signal measured during fMRIs. SMA01's lumbar spine was scanned, with the scans centered around the conus, twice pre-implant and once post-implant. Likewise, SMA02's lumbar spine was scanned twice pre-implant and twice post-implant. Acquisitions of the fMRIs of the spinal cord were focused on eliciting MN activation and recruiting proprioceptive afferents from specific leg muscles. This was achieved by conducting task-based functional scans measuring responses to an active task or a passive task. Neural activation was then compared across the spinal segments to find active voxels and to quantify the number of voxels that cross the thresholds for statistical significance to be considered active. The responses to both the active task and the passive task displayed an increase in the number of active voxels and z-score voxel values of anatomically relevant spinal levels, which indicates stronger neural activation signal in the spinal cord end-of-study compared to pre-study. The active task of the task-based functional scans involved the subject performing right leg extensions at a fixed rate when prompted to on a screen. This directly activates MNs in response to eliciting a controlled movement. As shown in FIGS. 23 and 24, three runs are acquired during each session. FIGS. 23A-23B illustrate neural activation results of SMA01 during the active task. FIG. 23A illustrates a bar graph representing the mean z-score of each session's fixed effects averaged runs during the active task. The black error bars represent S.E.M., and asterisks represent significance (p<0.001). FIG. 23B illustrates a histogram representing the number of active voxels at different z-score values for each session. Z-scores are thresholded at Z>2. As shown in FIG. 23B, the number of MNs and/or the firing rate of MNs increased post-implant, which is represented by the increased number of active voxels and their increased z-scores. FIGS. 24A-24B illustrate neural activation results of SMA02 during the active task. The results illustrated by FIG. 24A are comparable to those of FIG. 23A. FIG. 24B illustrates a histogram representing the number of active voxels at different z-score values for each session. Z-scores are thresholded at Z>2.5. Although the data in FIG. 24B is not statistically significant, it follows the same trend as SMA01's results as illustrated in FIG. 23A. Notably, the tail of the distribution of the z-scores is consistently larger post-explant than pre-implant.

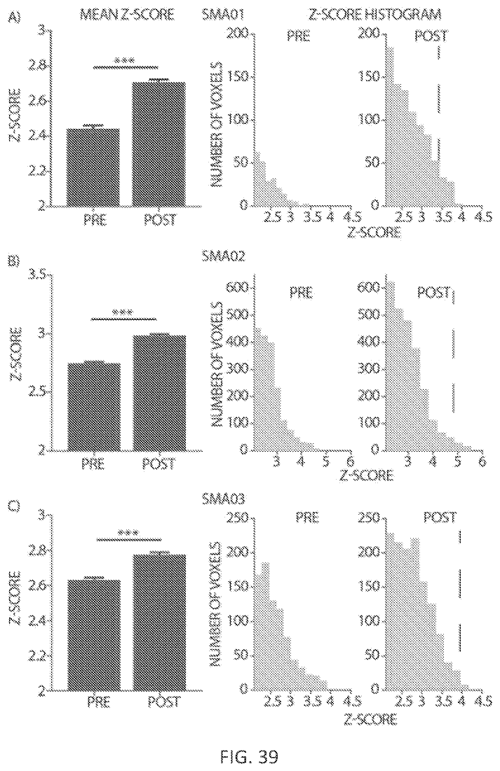

FIG. 39 illustrates the results of spinal cord fMRI during the active task (i.e., voluntary movements of the leg) for SMA01, SMA02, and SMA03. The z-scores are the statistical values that are used to determine whether a voxel has been activated or not. Activation in the spinal segments L1 to S1 that are targeted by the SCS therapy is calculated based on the z-scores. In all subjects, an increase in the number of activated voxels was observed, indicating that after four weeks of SCS treatment, the subjects' spinal circuits were more neurally active than their pre-study levels. The detailed methodology of fMRI is provided in the “Supplementary Data” section.

The passive task of the task-based functional scans involved mobilization of a limb by a physiotherapist. When a limb, such as the right leg, is mobilized, muscle spindles are recruited by stretching the muscles in which they are embedded. The physiotherapist extended the right knee joint at a fixed rate when prompted by an audio cue. As shown in FIG. 25, three runs were acquired during each session. FIGS. 25A-25B illustrate neural activation results of SMA01 during the passive task. FIG. 25A illustrates a bar graph representing the mean z-score of each session's fixed effects averaged runs during the passive task. FIG. 25B illustrates a histogram representing the number of active voxels at different z-score values for each session. Z-scores are thresholded at Z>2.5. As shown in FIG. 25B, the number of MNs and/or the firing rate of MNs has increased post-implant, which is represented by the increased number of active voxels and their increased z-scores. This may be due to the increased post-implant recruitment of proprioceptive afferents. In addition to the functional series T2 anatomical images and physiological (heart rate, respiratory) signals were acquired during each run. Data was analyzed using a general linear model.

### Observed Long-Term Effects

In this example, the long-term effects of SCS were measured in terms of MN resistance/recruitment, MN firing rates, maximum voluntary contraction, and overground walking gait. The measurements were taken several weeks after (e.g., 50+ days after) removal of the electrodes in a patient and illustrate changes in patients' motor ability after SCS has not been applied for several weeks. Each measurement is discussed below. It should be appreciated that different metrics can be used depending on the targeted muscle(s) and conditions of a patient.

Four weeks of SCS treatment combined with walking and strength training substantially and robustly improve strength, fatigue, and motor performances in all subjects. The magnitude of the long-term effects of SCS treatment exceeded those of all other known treatments for SMA. These changes correlate with electrophysiological and imaging signatures of changes occurring in the spinal cord, specifically the spinal MNs, and indicate that spinal MNs have decreased hyper-excitability and higher firing rates following SCS treatment. This suggests that SCS treatment is a disease-modifying intervention that changes the neural properties of SMA-affected motoneurons thereby improving motor performances. In general, all changes, both functional and electrophysiological, are inversely proportional to disease progression (i.e., larger effects in SMA01, who has less advanced disease progression, and smaller effects in SMA02, who has more advanced disease progression). These findings suggest that SCS treatment should be provided to SMA patients as soon as possible to maximize its effectiveness.

Although patients who were administered with an SMA therapy (SMA02 and SMA03, both being treated with Nusinersen) did not exhibit substantially better results than the patient who was not treated with a SMA therapy (SMA01) throughout the course of the four-week experiment, the effectiveness of the SMA therapy became apparent during the long-term follow-up to the experiment. The patient who was not on SMA therapy tended to exhibit long-term degradation in the beneficial effects of SCS. Conversely, the two patients who were on SMA therapy during the SCS surprisingly exhibited long-term stability in the beneficial effects of SCS. These findings indicate the better long-term effectiveness of SCS treatment with SMA therapy, in comparison with SCS without concurrent SMA therapy.

MN Resistance/Recruitment: The input resistance of SMA-affected MNs is abnormally high compared to healthy MNs, and, as a result, the SMA-affected MNs are over-excitable. This over-excitability makes SMA-affected MNs abnormally easy to recruit. By treating SMA-affected MNs with SCS, it is possible to lower the input resistance of the MNs such that they function more akin to healthy MNs.

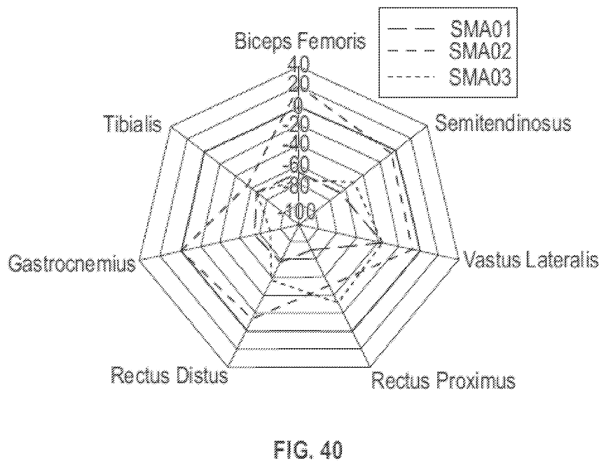

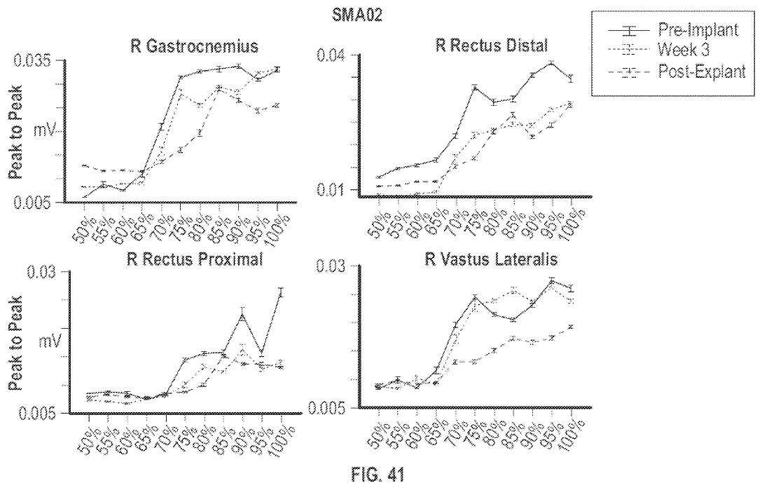

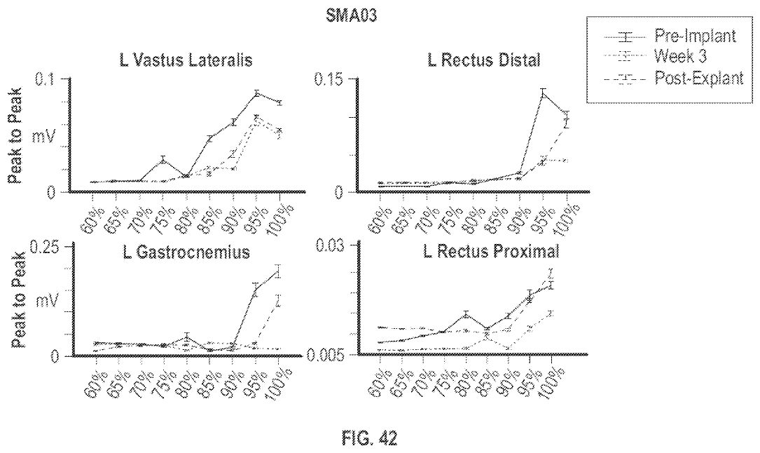

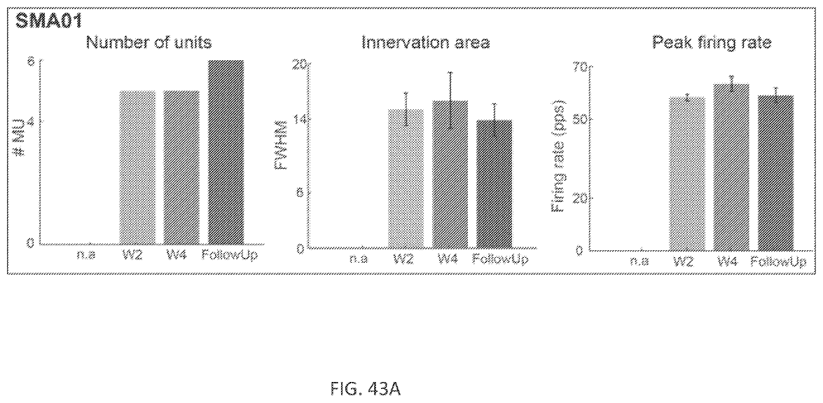

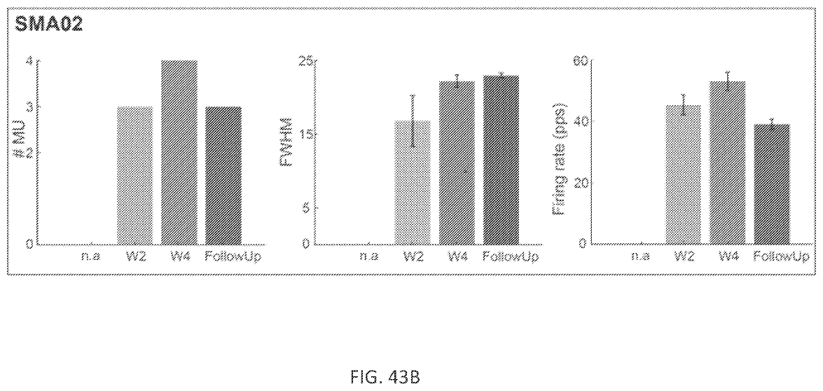

Transcranial magnetic stimulation (TMS) is a method of producing action potentials in MNs (and consequently, Motor Evoked Potentials (MEPs) in the muscles) by stimulating the cortico-spinal tract: i.e., a brain structure that connects human motor cortex to spinal MNs in humans. This pathway is independent from the spinal cord structures stimulated with SCS, and thereby offers a way to independently assess the excitability of spinal MNs by checking the strength of evoked MEPs with TMS pulses. FIG. 40 illustrates, using a web plot, the percentage of change in MEP peak-to-peak amplitude for SMA01, SMA02, and SMA03. The bolded heptagonal line represents the no-change line. Compared to pre-study values, all participants showed substantial reduction in MEP peak-to-peak. Changes were inversely proportional to disease progression, with SMA01 having the largest changes. The data indicates that spinal MNs in all subjects exhibit reduced excitability in response to SCS treatment. This corroborates the hypothesis that the changes observed in clinical scores, locomotion performances and/or strength are caused by the effects of SCS at the MN level. (FIGS. 41 and 42 illustrate how the peak-to-peak MEPs vary with pulse strength for various subjects.)

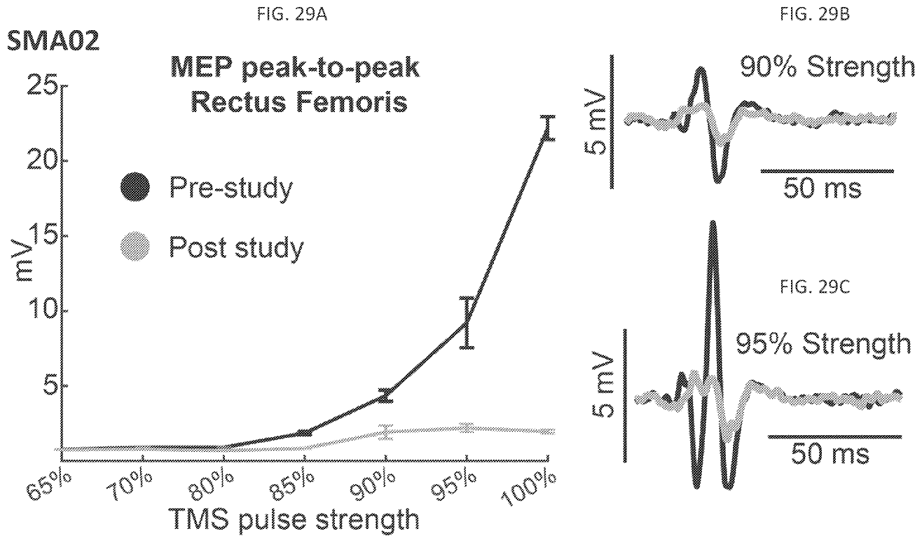

As shown in FIGS. 29A-29C, which illustrate data from SMA02, the MNs were harder to recruit post-study than they were pre-study for pulses of the same strength, and thus, input resistance of the MNs was greater post-study, meaning that, as a whole, the MNs function was more akin to healthy MNs after undergoing SCS treatment in combination with SMA therapy. As shown in FIG. 29A, the TMS-induced motor-evoked potential (MEP) of the rectus femoris muscle, as represented by peak-to-peak amplitude readings for various TMS pulse strengths, differed between pre-study and post-study measurements. The post-study amplitude readings were significantly lower than the pre-study amplitude readings, especially at stronger TMS pulse strengths (85%+). As shown in FIGS. 29B and 29C, which illustrate response amplitudes over time for a TMS pulse at 90% strength and 95% strength, respectively, the post-study readings have smaller peak-to-peak amplitudes than the pre-study readings. The changes in in pre- and post-study MN input resistance, as illustrated in FIGS. 29A-29C, are similar to the changes observed for SMA01 (not pictured).

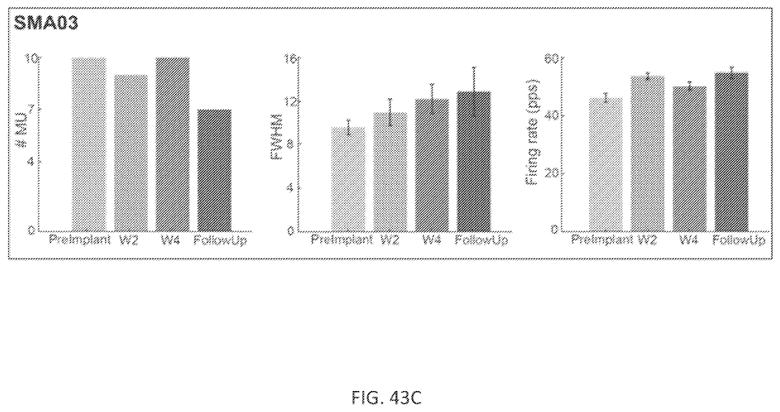

MN Firing Numbers/Rates: FIGS. 43A-43C illustrate single MN discharges from surface EMG signals of knee extensors and flexors muscles during isometric maximum voluntary contraction for SMA01, SMA02, and SMA03, respectively. Based on high-density EMG data, the number of observed MN units, their peak firing rates, and their “innervation area” (i.e., how much of the muscle is activated by a single MN) were calculated. Across data for all three subjects, no clear trend for MN unit number was detected over the duration of the study. This may be due, at least in part, to the fact that surface EMGs are not a reliable measure of true motoneuron numbers, as the number of detected units depends on electrode positioning as well as other tropic factors. Regarding peak firing rates, higher peak firing was observed in all three subjects at the end of the four-week study. Higher firing rates are indicative of the ability of MN units to increase strength when needed, correlating to changes in torques and demonstrating changes at the MN level. Innervation area is calculated by observing the size of each unit electric signature on a high-density electrode patch. Using this measure, a general mean increase in the size of detected MNs was observed over the course of the four-week study. This correlates with the increase in muscle strength observed for the subjects. In short, at the end of the SCS treatment study, the MNs exhibited faster firing rates, and their innervation territory on the muscle was larger.

FIGS. 26A-26B illustrate MN firing rates during maximum voluntary contraction for SMA01 at different points during and after the study, according to some embodiments. FIG. 26A, which plots the firing rate of two MNs (Unit 1 and Unit 2) alongside the maximum knee extension torque over time, illustrates how maximum firing rate is determined. FIG. 26B illustrates changes in MN firing rate during and after the study. Throughout the course of the SCS treatment study, i.e., from the second to the third week of the four-week experiment, the firing rate increases from below 60 peaks/second to over 64 peaks/second. Immediately following the end of the study, i.e., shortly after the SCS electrodes are explanted, the firing rate remained around 64 peaks/second, indicating that the benefits of SCS treatment (i.e., the increase in MN firing rate) are still present in this timeframe. However, in the long-term follow-up, i.e., several weeks after explant, the firing rate drops to around 60 peaks/second, indicating that the benefits of SCS treatment declined over time once the stimulation was removed. Thus, the immediate and end-of-study firing rate improvements associated with SCS treatment in SMA01, as illustrated in FIG. 26B, experienced long-term degradation. This long-term degradation is believed to be because SMA01 was not concurrently receiving an SMA therapy while undergoing SCS.

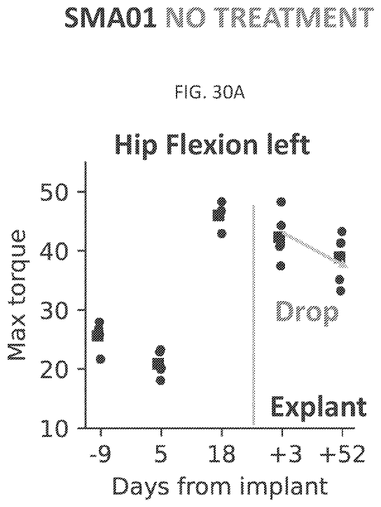

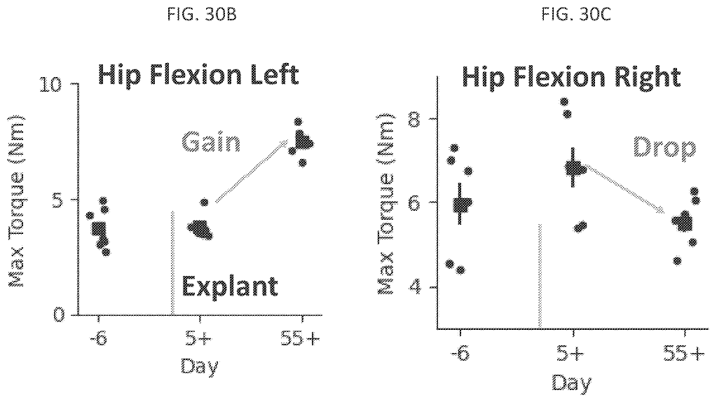

Maximum Voluntary Contraction: FIGS. 30A-30C illustrate long-term changes in torque measurements for SMA01 and SMA02. The dots correspond to the maximum torque produced during one single trial, and the square markers represent the mean across trials on the same day. FIG. 30A illustrates maximum left hip flexion torque measurements for SMA01 at different points before (−9 days), during (5 and 18 days), and after (+3 and +52 days) the four-week experiment. As shown, SMA01's MVC increased significantly over the course of the experiment (from −9 to 18 days) but decreased over the long-term (from +3 to +52 days after explant). Again, the immediate and end-of-study torque increases associated with SCS treatment in SMA01, as illustrated in FIGS. 7A-7E, 12A-12C, 13A-13C, 14A-14C, and 15A-15C, experienced long-term degradation, which is believed to be because SMA01 was not concurrently receiving SMA therapy while undergoing SCS.

In contrast to SMA01, SMA02 experienced improvements in muscle strength even after SCS treatment ended. FIG. 30B illustrates maximum left hip flexion torque measurements for SMA02 at different points before (−6 days) and after (+5 and +55 days) the experiment. As shown, SMA02's left hip MVC did not increase significantly over the course of the experiment (from −6 to +5 days), but unexpectedly, it increased significantly over the long-term (from +5 to +55 days after explant). Even after SCS treatment ended, the patient experienced improvements in muscle strength. However, this differs from FIG. 30C, which illustrates maximum right hip flexion torque measurements for SMA02 at different points throughout the experiment. As shown, SMA02's right hip MVC decreased over the long-term, even though his left hip MVC increased over the same time frame. Taken together, these figures suggest that, when coupled with SMA therapy, the beneficial effects of SCS may begin to be preserved for some muscles over the long-term.

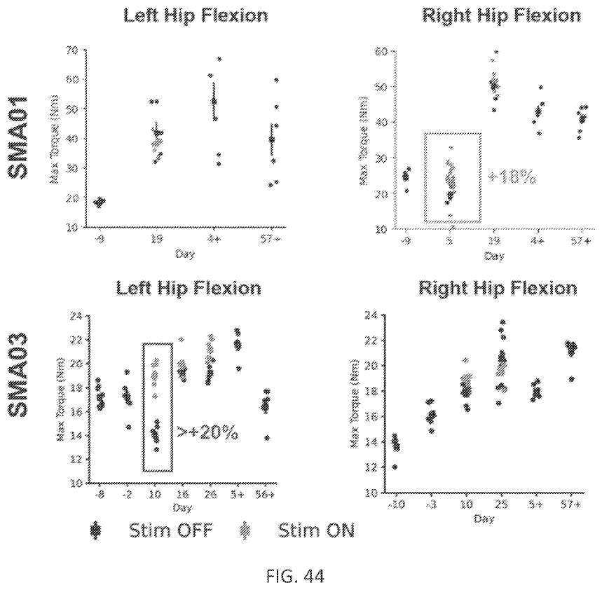

FIG. 44 illustrates long-term changes in isometric max torque measurements produced by left and right hip flexion for SMA01 and SMA03. The dots correspond to the maximum torque produced during one single trial, and the square markers represent the mean across trials on the same day. As shown in the figure, when SCS was applied during the early days of the study. SCS appeared to have a significant immediate effect relative to no SCS in some situations (e.g., right hip +18% with vs. without SCS on day 5 for SMA01, and left hip +>20% with vs. without SCS on day 10 for SMA03) but little immediate effect in other situations (e.g., left hip on day 19 for SMA01, right hip on day 19 for SMA01, left hip on days 16 and 26 for SMA02, and right hip on days 10 and 25 for SMA02). Thus, immediate effects of SCS were found only in the early days of the study and were not bilaterally present. After the end of the study, both subjects retained improvements to torque production even without SCS.

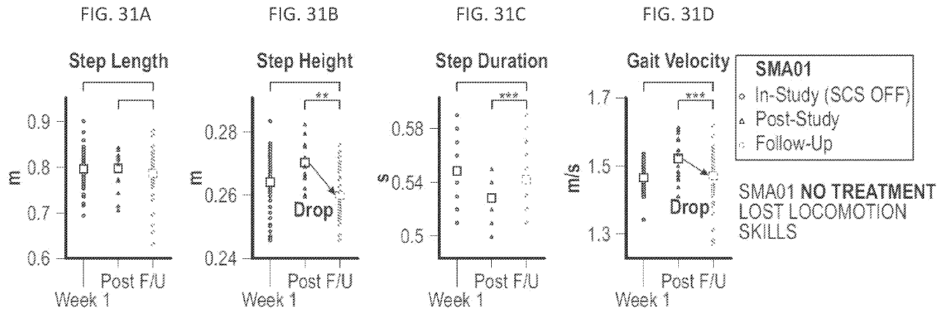

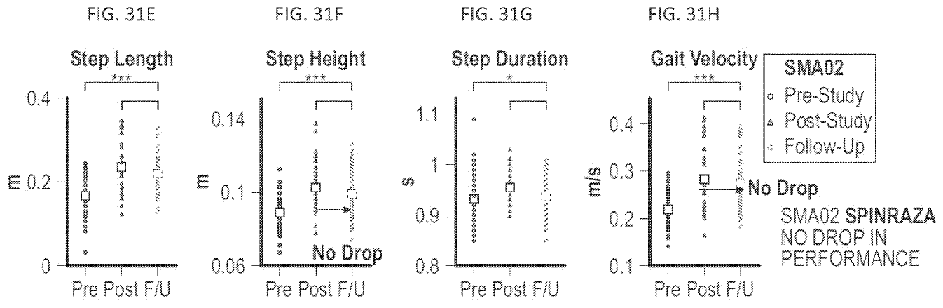

Overground Walking Gait: FIGS. 31A-31H illustrate long-term changes in several gait quality variables for SMA01 and SMA02. FIGS. 31A-31D show that, for SMA01, step length (FIG. 31A) and step duration (FIG. 31C) remained roughly the same over the long-term, but step height (FIG. 31B) and gait velocity (FIG. 31D) dropped significantly between the end-of-study measurement (within 1 week of explant) and the long-term measurement (50+ days after explant). These results indicate that, the long-term effects of SCS alone (without concurrent SMA therapy) may decline over time, and the patient's gait quality may suffer as a result. Conversely, FIGS. 31E-31H show that, for SMA02, step length (FIG. 31E), step height (FIG. 31F), step duration (FIG. 31G), and gait velocity (FIG. 31H) did not drop significantly between the end-of-study and long-term measurements. These results indicate that, when SCS is coupled with SMA therapy, the long-term effects of SCS may be preserved over time, and the patient's gait quality may be maintained at its end-of-study levels even after the SCS electrodes have been removed for several weeks.

Fatigue: Fatigue was quantified using the 6-meter walk test. For SMA02 and SMA03, the ratio between gait velocity of the last lap and the gait velocity of the first lap were used to quantify fatigue. For SMA01, who could perform a high number of laps, the ratio was calculated using the average of the first three and the last three laps.

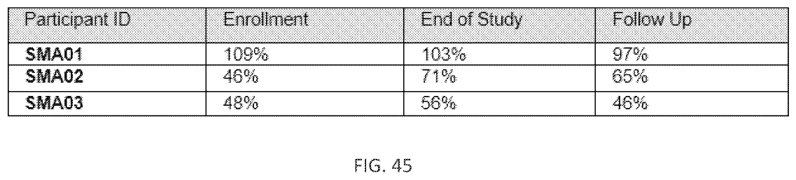

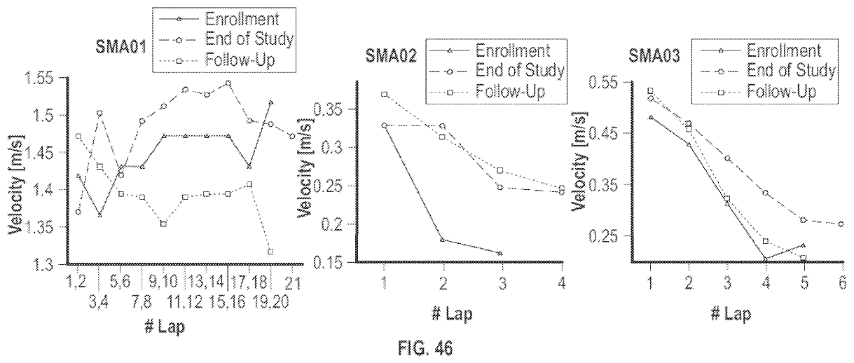

FIG. 45 illustrates a table of the fatigue values for SMA01, SMA02, and SMA03 before, during, and after the study. The fatigue values calculated express the drop in performances due to fatigue. For example, a value of 0.46 (as indicated by SMA02 at the start of the study) means that the velocity at the last lap(s) is only 46% of the velocity at the beginning of the test, indicating considerable fatigue. Overall, fatigue in SMA01 did not change significantly throughout the study. The fatigue value was close to 100%, indicating that SMA01 did not particularly suffer from fatigue in the 6MWT. Conversely, both SMA02 and SMA03 experience considerable fatigue at the start of the study (enrollment), around 40-50. Over the course of the study, the fatigue value of SMA02 increased significantly and remained elevated several weeks after follow-up. For SMA03, fatigue increased significantly during the study but dropped to pre-study values. However, the fatigue calculation is an estimate for quantifying fatigue and does not represent the complete picture of the benefits of SCS treatment. For example, FIG. 46 illustrates the velocities lap-by-lap for the 6MWT and shows clear improvement in lap-to-lap speed in all subjects.

### Clinical Outcome Tests

Clinical outcome tests were administered, including the manual muscle test, the 6-minute walk test, and the leg circumference test. Immediate, end-of-study, and long-term effects were observed across the clinical outcome tests, as discussed below.

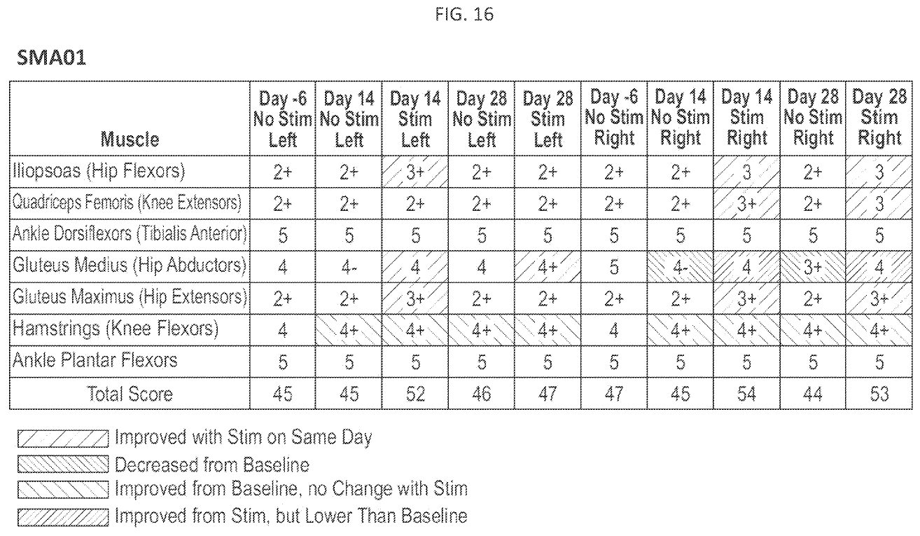

Manual Muscle Test: The manual muscle test (MMT) is a commonly accepted method of evaluating muscle strength. It individually measures the strength of each muscle and then sums their respective strength measurements to report a total score. The MMT was performed on SMA01 both pre-implant and on days 14 and 28 after implantation. The MMT showed consistent immediate effects from stimulation. Specifically, both the knee extensors/flexors and hip extensors/flexors showed improved scores with stimulation, as shown in the spreadsheet illustrated by FIG. 16, which contains manual muscle test scores for an SMA patient by muscle and session with and without stimulation.

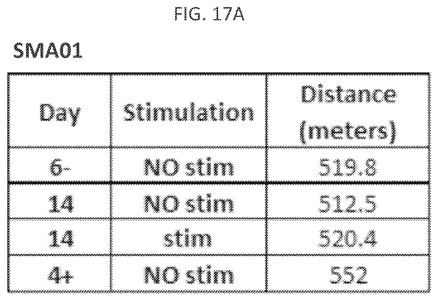

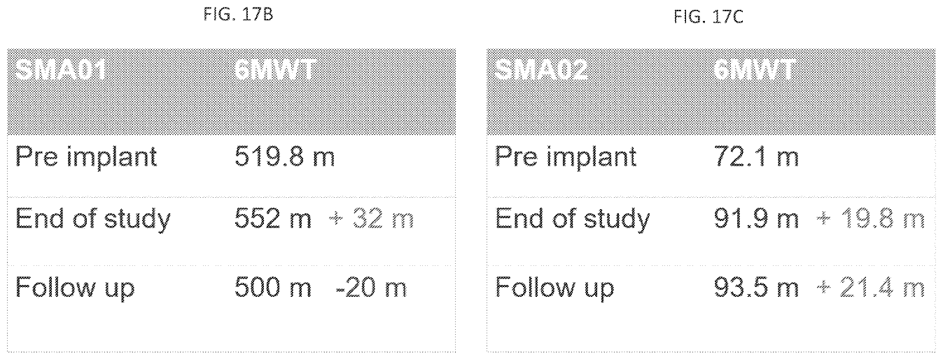

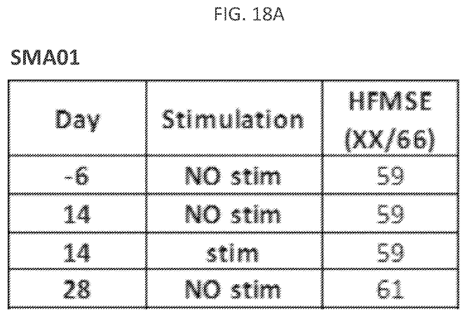

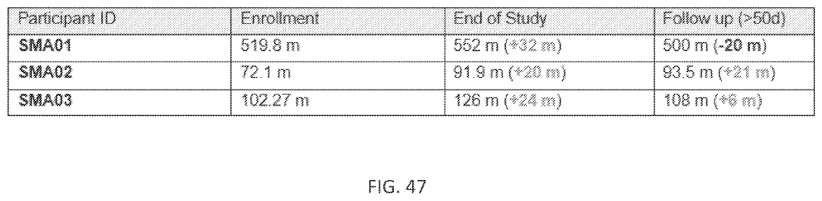

6-minute Walk Test: The 6-minute walk test (6MWT) was performed on SMA01. SMA02, and SMA03 pre-implant, on day 14 with and without stimulation, after explant of the electrodes, and over 50 days after explant. No significant immediate effects from stimulation were observed, but large improvements from the effects of stimulation in total distance traveled were observed at the end of the four-week experiment for all subjects, as shown in the tables illustrated by FIGS. 17A-17C and FIG. 47. Thus, the end-of-study effects of stimulation included increases in total distance traveled for both all subjects. However, the long-term effects of SCS differ between SMA01. SMA02, and SMA03. At the long-term measurement (50+ days after explant), the total distance traveled by SMA01 declined relative to his end-of-study results, as shown in FIG. 17B. Conversely, the total distance traveled by SMA02 during the long-term measurement increased slightly relative to his end-of-study results, as shown in FIG. 17C. The total distance traveled by SMA03 during the long-term measurement was lower than the end-of-study measurement but still higher than the initial measurements, as shown in FIG. 47. Taken together, this may indicate that when SCS is coupled with SMA therapy as in SMA02 and SMA03, the long-term effects of SCS may be preserved to some degree over time, and the increase in patient walking distance may be maintained to some degree even after the SCS electrodes have been removed for several weeks rather than degrade over time as they do in a patient where SCS is not coupled with SMA therapy such as SMA01.

FIG. 17A displays distances traveled by SMA01 during a 6MWT across different sessions, comparing stimulation off vs. stimulation on during the study (day 14). Expanding upon this data set, FIG. 17B displays distances traveled by SMA01 during a 6MWT across different sessions, specifically comparing pre-implant, end-of-study, and follow-up distances. Likewise, FIG. 17C displays distances traveled by SMA02 during a 6MWT across different sessions. FIG. 47 displays the same distances as FIGS. 17A-17C with additional distance information for SMA03.

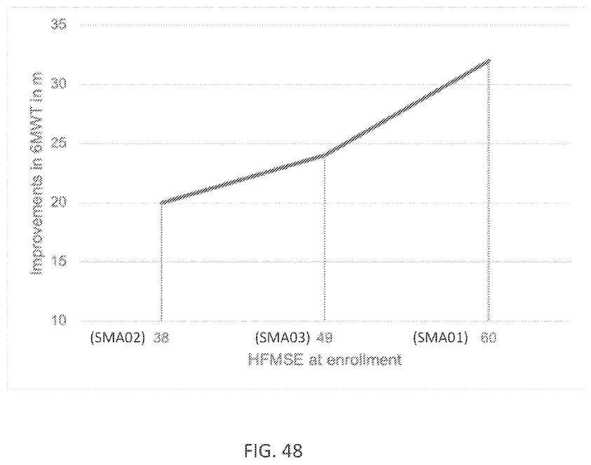

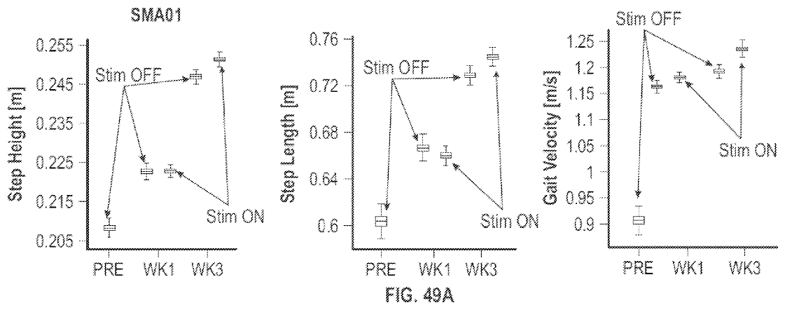

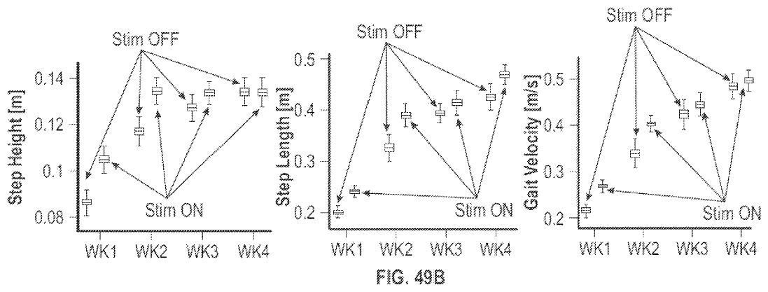

In existing literature, a clinical study has studied the impact of physical exercises on 6MWT in adults with ambulatory SMA. (Montes, J. et al. A randomized, controlled clinical trial of exercise in patients with spinal muscular atrophy: methods and baseline characteristics. J. Neuromuscul. Dis. 1, 151-161 (2014); Bartels, B., Montes, J., van der Pol, W. L. & de Groot, J. F. Physical exercise training for type 3 spinal muscular atrophy. Cochrane Database Syst. Rev. (2019).) The study found that after 6 months of physical exercise, participants improve by only 9 m in the 6MWT on average. In this experiment, at least twice that improvement was achieved for each subject, regardless of the severity of the subject's SMA, in a far shorter time span (four weeks), by incorporating SCS in addition to the physical exercise. Improvements in 6MWT were proportional to each subject's HFMSE at enrollment, but all were greater than 20 m, as shown in FIG. 48. Coincidentally, SMA02, who participated both in the exercise-only clinical study and the SCS-inclusive four-week experiment, gained 0 m in the 6MWT after 6 months of participation in the exercise-only clinical study, as opposed to 20 m in the SCS-inclusive four-week experiment.

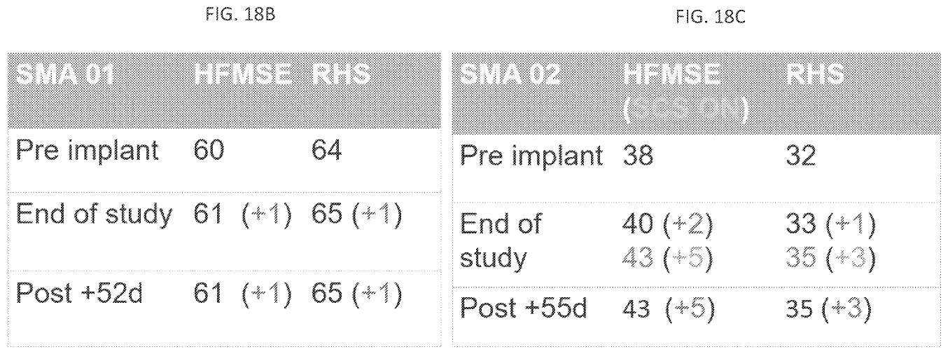

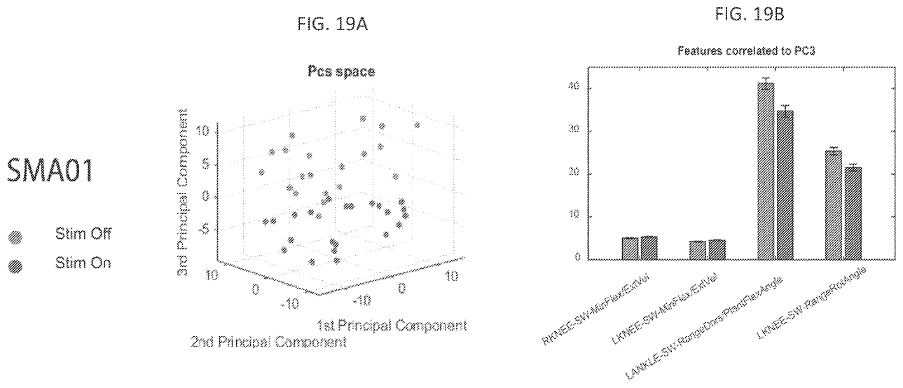

Clinical Scales, Hammersmith Functional Motor Scale Expanded (HFMSE) and Revised Hammersmith Scale (RHS): The HFMSE and RHS tests are validated instruments to assess the motor ability of children and adults with SMA Types 2 and 3. The results of HFMSE and RHS tests were measured before implant, at the date of explant (end-of-study), and at a long-term follow-up post-explant. The HFMSE test for SMA01 (without SMA therapy) showed small but relevant long-term effects with an increase in total score, achieving a final HFMSE score of 61. Improvements in the HFMSE can be generally very difficult to achieve for patients. Thus, even small changes (1-2 points in the present case) are indicative of improvements, as shown in the tables illustrated by FIGS. 18A and 18B, which contain HFMSE scores for SMA01. Likewise, as shown in the table illustrated by FIG. 18B, the RHS test for SMA01 shows a pre-implant RHS score of 64 and an explant RHS score of 65 (a 1 point increase). The increases in HFMSE and RHS scores remain consistent 52 days after explant, which is indicative of small but relevant long-term effects associated with the four-week experiment.

The HFMSE and RHS test scores for SMA02 (with SMA therapy) are illustrated in FIG. 18C, which shows similar long-term trends. The end-of-study scores increase by several points relative to SMA02's pre-implant scores, and this increase remains 55 days after explant, which is indicative of relevant long-term effects associated with the four-week experiment. Although the score increases for SMA02 are larger than those of SMA01, SMA02's baseline motor impairment is more severe than the impairment of SMA01. Taken together, the figures suggest that in both cases, patients may experience small but lasting improvements in motor ability that do not necessarily degrade over the long-term.

Leg Circumference: On day 18 after implantation, circumferences of SMA01's left and right legs were measured from 15.24 cm above the left and right patella, respectively. SMA01's left and right leg circumferences were measured to be 43.5 and 46 cm, respectively. On day 5 after the end of the four-week experiment and explant, the end-of-study left and right leg circumferences of the patient were measured to be 44 and 45 cm, respectively. These small differences may be considered insignificant.

### Supplemental Data

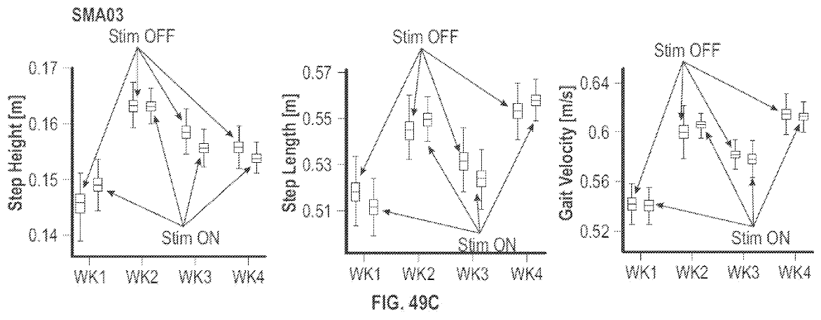

FIGS. 49A-49C provide overviews of gait changes for SMA01, SMA02, and SMA02, respectively, during the course of the four-week experiment. The subjects' performances between trials with and without SCS are illustrated for different weeks throughout the experiment. The performances are measured in terms of gait quality variables, specifically, step height, step length, and gait velocity, which are all calculated using full 3D limb kinematics. All subjects experienced a significant increase in all gait quality variables between the initial measurements and final measurements. The increase in the gait quality variables demonstrates substantial improvement in the quality of the subjects' gaits and indicates that, regardless of SMA severity, each patient experienced improvement in gait when treated with SCS. For each assessment, the immediate effects of SCS on vs. SCS off only slightly changes the subjects' performances; instead, the largest changes were observed in between the initial measurements and final measurements, indicating that SCS leads to long-term improvements in gait quality.

FIG. 35 illustrates the torques generated by SMA01 during maximum isometric contraction for a variety of movements. Each dot represents a single representation of the maximum voluntary contraction for each day of testing. The dots are vertically grouped based on when the testing occurred (e.g., pre-implant/week 1, post-study/week 4, and/or 6 weeks after the end of the study).

FIG. 36 illustrates the torques generated by SMA03 during maximum isometric contraction for a variety of movements. Each dot represents a single representation of the maximum voluntary contraction for each day of testing. The dots are vertically grouped based on when the testing occurred (e.g., pre-implant/week 1, post-study/week 4, and/or 6 weeks after the end of the study).

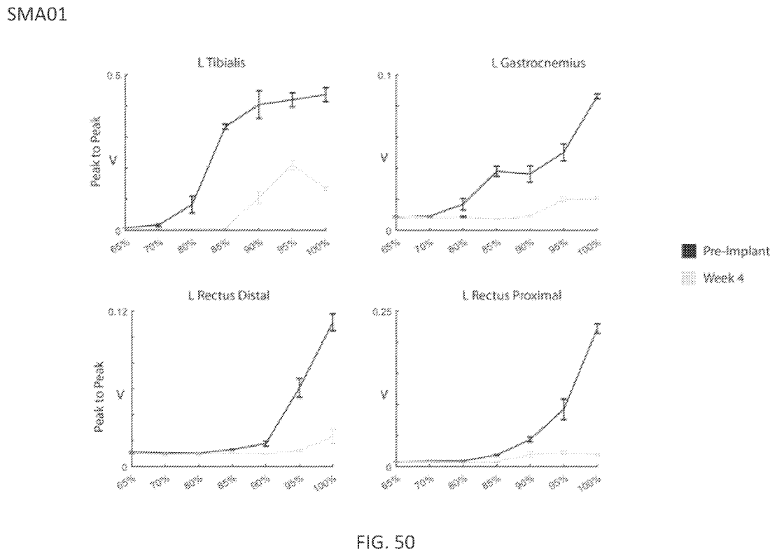

FIG. 50 illustrates TMS recruitment curves for SMA01 before implant and at end-of-study (week 4). Each datapoint represents the MEP peak to peak value for different stimulus intensities, which are plotted along the x-axis.

FIG. 41 illustrates TMS recruitment curves for SMA02 before implant, during week 3 of the study, and after explant. Each datapoint represents the MEP peak to peak value for different stimulus intensities, which are plotted along the x-axis.

FIG. 42 illustrates TMS recruitment curves for SMA03 before implant, during week 3 of the study, and after explant. Each datapoint represents the MEP peak to peak value for different stimulus intensities, which are plotted along the x-axis.

fMRI Data Acquisition: Participants were comfortably installed in the scanner (Siemens Prisma 3 Tesla) in a supine position. The spine coil was used. The participants were instructed to relax, to remain still, and to breathe normally. All three participants underwent at least three separate days of experimental recording, involving active limb mobilization to stretch specific groups of muscles. Functional acquisitions were performed using a gradient-echo echo-planar sequence with a ZOOMit field-of-view imaging, with repetition time (TR)=2.5 s, echo time (TE)=34 ms, FOV=48×144 mm, flip angle=80°, in plane resolution=1.0 mm×1.0 mm, and slice thickness=3 mm. 32 axial slices are acquired per volume. The bottom slice was placed at the end of the conus medullaris, located around the T12/L1 vertebra. Manual shimming adjustments focused on the spinal cord were done to adjust field homogeneity. Physiological data (respiratory and cardiac signals) are directly acquired using MRI compatible photoplethysmography and respiratory belt (for SMA02 and SMA03). A T2-weighted high-resolution anatomical image (sequence SPACE with a resolution of 0.4 mm×0.4 mm×0.8 mm, TR=1.5 s, TE=135 ms) was acquired for registration and normalization purposes.

Spinal Cord fMRI and Pre-processing: Lumbosacral spinal cord fMRI was performed to visualize the activation of the projectome of neurons innervating specific muscles. The fMRI pre-processing, processing and analysis pipeline was based on recent cervical and lumbar spinal cord fMRI studies. (Rowald, A., Komi, S., Demesmaeker, R. et al. Activity-dependent spinal cord neuromodulation rapidly restores trunk and leg motor functions after complete paralysis. Nat Med 28, 260-271 (2022); Kinany N, et al. Dynamic functional connectivity of resting-state spinal cord fMRI reveals fine-grained intrinsic architecture. Neuron 2020; 108 (3): 424-435 e4; Kinany, N., Pirondini, E., Martuzzi, R., Mattera, L., Micera, S., Van de Ville, D., 2019. Functional imaging of rostrocaudal spinal activity during upper limb motor tasks. Neuroimage 200, 590-600.) The pipeline was tailored to image the lumbar spinal cord. The protocol involved 3 runs for active leg mobilization. Each run had active blocks of 16 s where the participant performed knee extensions at a rate of 0.5 Hz. One run consists of 9 active blocks, and in total lasted 6 minutes.

The fMRI pre-processing was carried out using the FMRIB Software Library (FSL) v5.0.15 and the Spinal Cord Toolbox (SCT) v5.0. Motion correction was performed using SCT toolbox. The mean functional image was used to automatically detect the centerline of the spinal cord. A cylindrical mask (diameter of 30 mm) along the centerline was generated to prevent the inclusion of regions moving independently from the spinal cord and slice-wise realignment was performed with the mean functional image as reference. All runs corresponding to the same session in the scanner were aligned to the first run of the session using three-dimensional rigid body realignment (spline interpolation and least square cost function). All images were inspected to ensure that any artefacts with insufficient signal were cropped out. Motion scrubbing was also performed with FSL's tool to identify outlier volumes, using DVARS (the root mean square of the difference of intensity between 26 consecutive volumes) metric in the spinal cord, with a box-plot cut-off (75th percentile+1.5×the interquartile range). Both the cerebrospinal fluid and the spinal cord were automatically segmented (with manual corrections when necessary) using SCT from the mean functional image. Nuisance regressors were built using FSL's physiological noise modelling tool on the acquired cardiac and respiratory signals, using an approach based on the RETROICOR procedure. Low and high order Fourier expansions were used to model the physiological signals which results in 32 noise regressors, to which an additional cerebrospinal fluid regressor is included (10% most variable cerebrospinal fluid voxels). These 33 physiological noise regressors (PNM and CSF) were combined with motion correction parameters (i.e., two slice-wise regressors, for the motion in x and y) and motion outliers and regressed from the fMRI time-series using FSL's fMRI Expert Analysis Tool (FEAT). The resulting residuals were then spatially smoothed using a 3D Gaussian kernel with a full width half maximum (FWHM) of 2×2×6 mm³. Smoothing is performed along the centerline of the spinal cord, so as to preserve anatomical consistency. Finally, acquisition timings corresponding to the task-design, were submitted to a specific first level generalized linear model. A second level fixed effects analysis (subject level) was performed by combining the three runs. Whenever possible, multiple comparison corrections are performed (Z>1.5, p<0.05), and if not, possible maps are uncorrected (Z>1.5, p<0.05). Then maps are averaged for pre implant or post implant.

Spinal segments L1 to S2 were identified using the high-resolution structural MRI. The L1 dorsal root was identified from its entry region in the spinal canal (entering just below the L1 vertebra) until the region where it innervates the spinal cord, which defines the L1 spinal segment. The more caudal segments (L2-S2) were identified by following the dorsal roots along the rostrocaudal axis. The window of analysis was focused between a single vertebral level and on root levels involved in leg extension movement.

### Additional Discussion of Subjects SMA01, SMA02, and SMA03

Over the weeks of testing the subjects, there were noticed marked changes of apparent sudden increase in the maximal forces that the patients were producing even without stimulation from one week to another. The torque changes over time were investigated and surprisingly large increases were found in almost all leg muscles, and particularly at the hip flexors and extensors in all participants, but also in knee extensors and flexors. These changes started to appear from week 2 and significantly picked up from week 3. Assessing across all joints movements, the changes appeared to be focused on the hip muscles, which were directly targeted with stimulation protocols, are the most vulnerable to SMA, and were less trained in the exercises that focused on the knee. Significantly, these improvements vastly exceed the size of assistive effects and led over time to appreciable motor benefits. For example, with assistive effects mounting on top of the newly acquired strength, SMA03 could stand up from hinging on a desk by week 4, a task that the participant was not able to do prior to the beginning of the study and without SCS. In summary, it was found that measurable assistive effects on strength resulted when SCS was turned on, and these effects were paralleled by large changes in strength without stimulation that suddenly appeared over four weeks.

Improvements in fatigue and walking were also observed. Despite the large difference in initial deficits of the participants, all three significantly improved over the 4 weeks on the gait variables of step height, step length and gait velocity. Importantly, it was observed that when SCS was turned on, there were further improvements in step height, step length and gait velocity, determining visible changes in gait patterns even in the same session. SMA02 in particular experienced prominent improvements in gait quality-they had severe muscle degeneration and prior to the trial did not flex their knee at all, but after treatment their gait was completely changed and they became able to fully flex the knee. Changes in kinematics were paralleled by immediate increases in EMG power when SCS was turned on, as well as increases in modulation of EMG activity over time, signaling that kinematics movements were caused by changes in muscle activation. The standardized 6MWT was used to assess improvements on fatigue, and it was surprisingly found that all three participants improved more than 20m in the 6MWT in only 4 weeks (compared to a minimally important clinical difference of 14m). Furthermore, there was an improvement in both 6MWT and fatigue, whereas previous reports show disassociation between these two factors. This demonstrates an intrinsic change in the motor syndromes of the participants in this study as a result of the SCS.

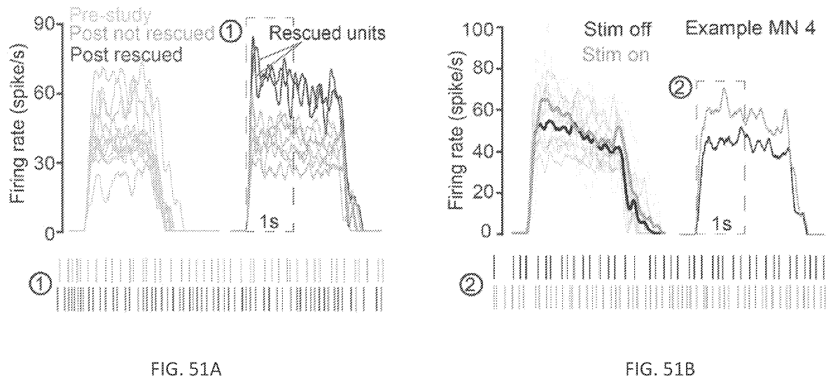

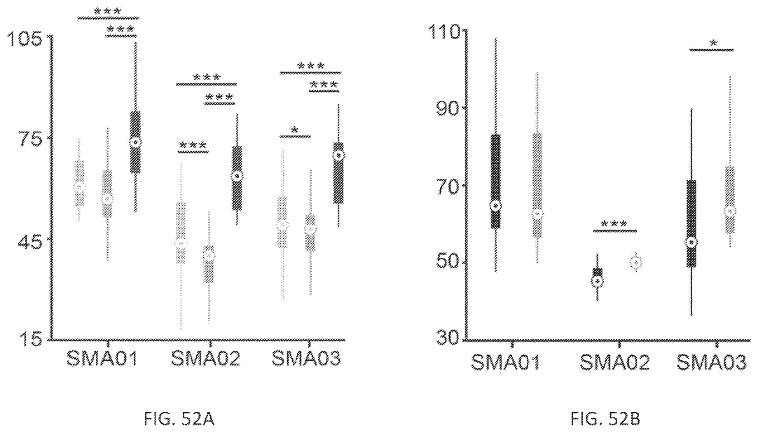

The size and rapidity of the observed improvements in force and motor control indicated they may be caused by changes in neural drive rather than muscle mass. To evaluate this, High-Density Electromyography (HDEMG) was used during maximal voluntary contractions, and single motor unit discharges were extracted. It was found that when SCS was turned on, motor units increased their overall firing rates. When motoneuron firing rates pre-vs. post-study were compared, unit firing dynamics was quite consistent among units pre-study when producing isometric forces. However, post-study new units emerged that appeared to have a markedly different behavior. These units showed significantly higher firing rate bursts at the onset of the force that correlated with higher-peak values obtained experimentally (FIG. 51A). The distribution of firing rates for this peak was markedly different from any other unit recorded pre-study, and these units were labeled “rescued units” since they demonstrated the acquired ability to fire significantly higher. These rescued units with remarkably similar behavior were found in all three participants. FIGS. 51A and 51B provide traces of single unit motoneuron firing rate during a maximum voluntary contraction in isometric conditions. Rescued motoneurons are defined as those whose mean peak firing rate across repetitions (n=6) were above the 99.7 percentile of the mean peak firing rate during the pre-study sessions. Raster plots show the spike times of two example motoneurons. FIG. 51A shows pre and post examples while FIG. 51B shows stim on vs stim off. FIGS. 52A and 52B provide quantification of the mean peak firing rates across all isometric conditions for pre vs post (FIG. 52A) and stim on vs stim off (FIG. 52B), for each of the three patients.

Additional details of the clinical trial can be found with the Clinicaltrials.gov Identifier NCT05430113, “Spinal Cord Stimulation in Spinal Muscular Atrophy (SCSinSMA),” available at https://clinicaltrials.gov/ct2/show/NCT05430113.

### Example 3

Spinal Cord Stimulation to Treat Upper Limb Muscle Deficiencies in Subjects with SMA

In this study, patients with SMA will be recruited and the cervical spinal cord stimulated using SCS.

Prior to implantation of the electrodes, each subject will undergo a detailed history and physical examination, and a detailed musculoskeletal and neurologic examination. The Physical Activity Disability Survey (PADS) and Revised Hammersmith Functional Scale (RHS) will be performed, as well as an evaluation of the ability of the subject to stand independently for at least 3 seconds. Pre-operative high resolution MRIs will be performed, and imaging obtained will include high-resolution T1 weighted images, high-resolution T2 weighted images, and Diffusion Tensor Imaging (DTI). The same brain images will be acquired also at the end of the study to document possible structural changes in fiber tracts that might correlate with brain plasticity and motor recovery. A spinal cord functional Magnetic Resonance at rest and during muscle contractions will also be obtained, and the same images will be acquired at the end of the study to document possible changes in functional connectivity of the brain and spinal cord that might correlate with plasticity and motor recovery.

To carry out the SCS, 2 to 4, octopolar Medtronic leads will be temporarily implanted in the epidural space of the C4 to T1 spinal vertebra of each patient. This section of the spinal vertebra includes nerve roots innervating muscles of the trunk, back and upper limbs. The device will be tunneled percutaneously through the skin and secured in place with tape, and externalized for a maximum duration of 29 days. During the SCS testing activities, the leads will be connected to an external stimulator. Examples of external stimulators that may be used are the Natus Medical Protektor32, the Digitimer DS8, or the external wireless stimulator of the Intellis system from Medtronic.

After implantation, each subject will participate in a series of electrophysiological stimulation trials. The stimulation parameters will be varied but will remain within: pulse amplitude between 0.2 and 10 mA; pulse frequency between 0.1 and 500 Hz; and pulse width between 100 and 400 μs. EMG responses and joint kinematics will be recorded in response to stimulation trains and the subject will be asked to respond to a set of standard psychophysical questions to assess possible levels of discomfort, as well as to provide any additional comments. Patients will also participate in various motor task evaluations to measure the impact SCS has on upper limb function, including deltoids, biceps, triceps and wrist extensors flexors of the arms.

KINARM System: Since SCS recruits proprioceptive afferents in the dorsal roots, it is important to assess the ability of each subjects to consciously and unconsciously process proprioceptive feedback. Each subject's active proprioception capacity will be evaluated by performing arm movement assessments in the KINARM system (Kinarm, Kingston, ON, Canada) at baseline, day 29, and follow up visits. The KINARM is a human research intended system used to evaluate motor performances in subjects with stroke. The participant will be seated in a chair with their arms supported against gravity. The participant will be asked to actively perform isotonic elbow extension and flexion, e.g. movements, against an opposing constant force of 0%, 10%, 20%, and 30% of the maximum voluntary surface electromyogram (EMG). Surface EMG from the main arm muscles will be recorded. Throughout the experiment the participant's arm and hand will be occluded from vision with a protective screen built in in the KINARM system. The capacity of each patient to assess limb position and joint movement will be assessed.

HUMAC NORM by CSMi: To quantify the range of motion of arm joints and the joint torques produced with and without SCS, each patient will be assessed using the clinical isokinetic testing system: HUMAC NORM by CSMi. This data may be captured at baseline, during the 29 days of the implantation period, and at the follow up visit.

Hand Bike: Impact of SCS will also be evaluated using a hand bike. Periodically, patients will be asked to use a hand bike for as long as they can. During these trials SCS will be provided to verify its effects on motor task execution. Subjects will use the hand bike at their preferred speed and resistance while they are receiving SCS or in control trials without stimulation. Parameters evaluated will include torque asymmetries between arms, time, distance, and velocity. This evaluation may occur pre-implantation (baseline), during implantation, and in the follow-up phase after the implant is removed to evaluate the impact of SCS on upper limb functions.

Three-dimensional Reaching/Simulated Activities of Daily Living (ADL) Task: Upper limb function will also be evaluated through the performance of a three-dimensional reaching/simulated activities of daily living (ADL) task. Patients will be asked to reach towards custom built objects and grasp them. Each object will promote a particular type of grasp. For example, a large sphere will promote a power whole hand grasp, a cylinder will promote a cylindrical grasp while a flat shape object will promote a precision index-thumb pinch grasp. Objects may be placed in space by haptic robots that can measure grasping and interaction forces.

Primary outcomes related to muscle strength will be assessed pre-study, at week 2, week 4 and post-study follow up (at least 4 weeks after completion). SCS leads will be removed no later than 29 days after device placement.

### Enumerated Embodiments

The disclosure will now be further described by the following numbered embodiments which are to be read in connection with the preceding paragraphs, and which do not limit the disclosure. The features, options and preferences as described above apply also to the following embodiments.

Embodiment 1. A method for treating spinal muscular atrophy (SMA) in a subject, comprising: applying a therapeutically effective amount of an electrical stimulus to sensory neurons innervating a body region of the subject with a motor impairment due to SMA, wherein the electrical stimulus is applied with one or more electrodes controlled by a neurostimulator, and wherein application of the electrical stimulus treats the motor impairment due to SMA in the subject.

Embodiment 2. The method of Embodiment 1, wherein applying the electrical stimulus increases the firing rate probability of spinal motoneurons innervating the body region of the subject with the motor impairment due to SMA.

Embodiment 3. The method of Embodiment 1 or Embodiment 2, wherein the stimulation is applied at or below a motor threshold such that the stimulation does not directly elicit movement and/or muscle activity of the body region of the subject with the motor impairment due to SMA.

Embodiment 4. The method of any one of Embodiments 1-3, wherein the electrical stimulus comprises electrical pulses having an amplitude of about 10 μA to about 100 mA, a width between about 40 μs and about 2 ms, and a frequency of about 10 Hz to about 2000 Hz.

Embodiment 5. The method of any one of Embodiments 1-4, wherein the electrical stimulus comprises electrical pulses having an amplitude of about 10 μA to about 10 mA, a width between about 40 μs and about 2 ms, and a frequency of about 10 Hz to about 1000 Hz.

Embodiment 6. The method of any one of Embodiments 1-5, wherein the electrical stimulus comprises electrical pulses having an amplitude of about 100 μA to about 10 mA, a width between about 40 μs and about 500 μs, and a frequency of about 10 Hz to about 1000 Hz.

Embodiment 7. The method of any one of the prior Embodiments, wherein the electrical stimulus comprises a stimulation pattern of a series of 2 to 5 pulses separated by inter-pulse intervals of about 3 ms to about 10 ms and wherein the series is repeated at a frequency of about 10 Hz to about 100 Hz.

Embodiment 8. The method of Embodiment 7, wherein the stimulation pattern is a series of 3 pulses separated by inter-pulse intervals of about 5 ms, wherein the series is repeated at a frequency of about 30 to about 100 Hz, and wherein the pulse width is about 200 μs.

Embodiment 9. The method of any one of Embodiments 4-8, wherein the pulses are cathodic-first biphasic or monophasic charge balanced pulses.

Embodiment 10. The method of any one of the prior Embodiments, wherein electrical stimulation is applied for at least 2 hours/day over a period of at least 6 months.

Embodiment 11. The method of any one of Embodiments 1-9, wherein electrical stimulation is applied for at least 1 hour/day over a period of at least 1 month.

Embodiment 12. The method of any one of the prior Embodiments, wherein the one or more electrodes are contained within an array of independently controllable electrodes implanted in the subject.

Embodiment 13. The method of Embodiment 12, wherein the electrode array is a multi-electrode paddle array.

Embodiment 14. The method of any one of the prior Embodiments, wherein the one or more electrodes are implanted epidurally at the spinal cord of the subject.

Embodiment 15. The method of any one of Embodiments 1-14, wherein the one or more electrodes are implanted at the dorsal rootlets of the one or more sensory neurons innervating the body region with the motor impairment of the subject.

Embodiment 16. The method of any one of Embodiments 1-14, wherein the one or more electrodes are implanted at the dorsolateral aspect of the spinal cord adjacent to the dorsal roots of the one or more sensory neurons innervating the body region with the motor impairment of the subject.

Embodiment 17. The method of any one of Embodiments 1-13, wherein the one or more electrodes are implanted at the dorsal root ganglia of the one or more sensory neurons innervating the body region with the motor impairment of the subject.

Embodiment 18. The method of any one of Embodiments 1-12, wherein the one or more electrodes are contained in a cuff that surrounds a peripheral nerve containing the sensory neurons innervating the body region of the subject with the motor impairment due to SMA.

Embodiment 19. The method of any one of Embodiments 1-12, wherein the one or more electrodes penetrate a peripheral nerve or dorsal root ganglion containing the sensory neurons innervating the body region of the subject with the motor impairment due to SMA.

Embodiment 20. The method of any one of the prior Embodiments, wherein the body region of the subject is selected from at least one of lower back, hip, leg, ankle, and foot; and the one or more electrodes are implanted at the dorsolateral aspect of the spinal cord and span one or more of the T11-S1 nerve roots.

Embodiment 21. The method of any one of the prior Embodiments, wherein the body region of the subject is selected from at least one of upper arm, shoulder, arm, hand, and respiratory muscles; and the one or more electrodes are implanted at the dorsolateral aspect of the spinal cord and span one or more of the C3-T2 nerve roots.

Embodiment 22. The method of any one of the prior Embodiments, wherein the body region of the subject is selected from at least one of the chest, chest wall, abdomen, upper back, and middle back; and the one or more electrodes are implanted at the dorsolateral aspect of the spinal cord and span one or more of the T3-T10 nerve roots.

Embodiment 23. The method of any one of the prior Embodiments, wherein the neurostimulator is an external or implanted pulse generator.

Embodiment 24. The method of any one of the prior Embodiments, further comprising implanting the neurostimulator in the subject.

Embodiment 25. The method of any one of the prior Embodiments, further comprising selecting the subject with SMA for treatment.

Embodiment 26. The method of any one of the prior Embodiments, wherein the motor impairment comprises partial or complete paralysis, loss of dexterity, loss of muscle strength, and/or uncontrollable muscle tone.

Embodiment 27. The method of any one of the prior Embodiments, wherein the locations of the one or more electrodes are chosen based on the body region of the subject with the motor impairment due to SMA.

Embodiment 28. The method of Embodiment 27, wherein the locations of the one or more electrodes are determined by: stimulating a plurality of contact points in the subject to target at least one muscle within the body region of the subject with the motor impairment due to SMA; measuring the electrical activity associated with said muscle in response to the stimulation; and identifying one or more contact points in the plurality of contact points as locations of the electrodes based on said electrical activity.

Embodiment 29. The method of any one of the prior Embodiments, wherein at least one of the one or more electrodes is placed transcutaneously.

Embodiment 30. The method of any one of the prior Embodiments, wherein at least one of the one or more electrodes is implanted.

Embodiment 31. A method of stimulating one or more motoneurons impaired by SMA, said method comprising applying an electrical stimulus to at least one of said motoneurons wherein said motoneurons innervate a body region of a subject with a motor impairment due to SMA.

Embodiment 32. A method for treating spinal muscular atrophy (SMA) in a subject, comprising applying a therapeutically effective amount of an electrical stimulus to sensory neurons innervating a body region of the subject with a motor impairment due to SMA.

Embodiment 33. A method of increasing firing rate of motoneurons impaired by SMA in a subject, comprising applying a therapeutically effective amount of an electrical stimulus to sensory neurons innervating a body region of the subject to increase the motoneuron firing rate of the subject.

Embodiment 34. The method of Embodiment 33, wherein the subject has a first motoneuron firing rate before the application of the therapeutically effective amount of the electrical stimulus and a second motoneuron firing rate after the application.

Embodiment 35. The method of Embodiment 33 or Embodiment 34, wherein the electrical stimulus comprises electrical pulses having an amplitude of about 10 μA to about 100 mA, a width between about 40 μs and about 2 ms, and a frequency of about 10 Hz to about 2000 Hz.

Embodiment 36. The method of any one of Embodiments 33-35, wherein the electrical stimulus is applied for at least 1 hour/day over a period of at least 1 month.

Embodiment 37. The method of any one of Embodiments 33-36, wherein the motoneuron firing rate of the subject is computed from electrical signals produced by the motoneurons while varying the parameters of the electrical stimulus.

Embodiment 38. The method of any one of Embodiments 33-37, wherein increasing firing rate of motoneurons impaired by SMA increases joint torques and muscle strength of the subject.

Embodiment 39. A method of increasing excitability of motoneurons impaired by SMA in a subject to a sensory afferent input, comprising applying a therapeutically effective amount of an electrical stimulus to sensory neurons innervating a body region of the subject to increase the motoneuron excitability of the subject.

Embodiment 40. The method of Embodiment 39, wherein the subject has a first motoneuron excitability before the application of the therapeutically effective amount of the electrical stimulus and a second motoneuron excitability after the application.

Embodiment 41. The method of Embodiment 39 or Embodiment 40, wherein the electrical stimulus comprises electrical pulses having an amplitude of about 10 μA to about 100 mA, a width between about 40 μs and about 2 ms, and a frequency of about 10 Hz to about 2000 Hz.

Embodiment 42. The method of any one of Embodiments 39-41, wherein the electrical stimulus is applied for at least 1 hour/day over a period of at least 1 month.

Embodiment 43. The method of any one of Embodiments 39-42, wherein the motoneuron excitability of the subject is computed from electrical signals produced by the motoneurons while varying the parameters of the electrical stimulus.

Embodiment 44. The method of any one of Embodiments 39-43, wherein increasing excitability of motoneurons impaired by SMA increases joint torques and muscle strength of the subject.

Embodiment 45. A therapeutically effective amount of an electrical stimulus to sensory neurons innervating a body region of the subject with a motor impairment due to SMA for treating SMA in a subject, wherein the electrical stimulus is to be applied with one or more electrodes controlled by a neurostimulator, and wherein application of the electrical stimulus treats the motor impairment due to SMA in the subject.

Embodiment 46. The electrical stimulus for use of Embodiment 45, wherein applying the electrical stimulus increases the firing rate probability of spinal motoneurons innervating the body region of the subject with the motor impairment due to SMA.

Embodiment 47. The electrical stimulus for use of Embodiment 45 or Embodiment 46, wherein the stimulation is applied at or below a motor threshold such that the stimulation does not directly elicit movement and/or muscle activity of the body region of the subject with the motor impairment due to SMA.

Embodiment 48. The electrical stimulus for use of Embodiments 45-47, wherein the electrical stimulus comprises electrical pulses having an amplitude of about 10 μA to about 100 mA, a width between about 40 μs and about 2 ms, and a frequency of about 10 Hz to about 2000 Hz.

Embodiment 49. The electrical stimulus for use of Embodiments 45-48, wherein the electrical stimulus comprises electrical pulses having an amplitude of about 10 μA to about 10 mA, a width between about 40 μs and about 2 ms, and a frequency of about 10 Hz to about 1000 Hz.

Embodiment 50. The electrical stimulus for use of Embodiments 45-49, wherein the electrical stimulus comprises electrical pulses having an amplitude of about 100 μA to about 10 mA, a width between about 40 μs and about 500 μs, and a frequency of about 10 Hz to about 1000 Hz.

Embodiment 51. The electrical stimulus for use of any one of the prior Embodiments, wherein the electrical stimulus comprises a stimulation pattern of a series of 2 to 5 pulses separated by inter-pulse intervals of about 3 ms to about 10 ms and wherein the series is repeated at a frequency of about 10 Hz to about 100 Hz.

Embodiment 52. The electrical stimulus for use of Embodiment 51, wherein the stimulation pattern is a series of 3 pulses separated by inter-pulse intervals of about 5 ms, wherein the series is repeated at a frequency of about 30 to about 100 Hz, and wherein the pulse width is about 200 μs.

Embodiment 53. The electrical stimulus for use of any one of Embodiments 48-52, wherein the pulses are cathodic-first biphasic or monophasic charge balanced pulses.

Embodiment 54. The electrical stimulus for use of any one of the prior Embodiments, wherein the electrical stimulus is applied for at least 2 hours/day over a period of at least 6 months.

Embodiment 55. The electrical stimulus for use of any one of Embodiments 45-53, wherein the electrical stimulus is applied for at least 1 hour/day over a period of at least 1 month.

Embodiment 56. The electrical stimulus for use of any one of the prior Embodiments, wherein the one or more electrodes are contained within an array of independently controllable electrodes implanted in the subject.

Embodiment 57. The electrical stimulus for use of Embodiment 56, wherein the electrode array is a multi-electrode paddle array.

Embodiment 58. The electrical stimulus for use of any one of the prior Embodiments, wherein the one or more electrodes are implanted epidurally at the spinal cord of the subject.

Embodiment 59. The electrical stimulus for use of any one of Embodiments 45-58, wherein the one or more electrodes are implanted at the dorsal rootlets of the one or more sensory neurons innervating the body region with the motor impairment of the subject.

Embodiment 60. The electrical stimulus for use of any one of Embodiments 45-58, wherein the one or more electrodes are implanted at the dorsolateral aspect of the spinal cord adjacent to the dorsal roots of the one or more sensory neurons innervating the body region with the motor impairment of the subject.

Embodiment 61. The electrical stimulus for use of any one of Embodiments 45-57, wherein the one or more electrodes are implanted at the dorsal root ganglia of the one or more sensory neurons innervating the body region with the motor impairment of the subject.

Embodiment 62. The electrical stimulus for use of any one of Embodiments 45-56, wherein the one or more electrodes are contained in a cuff that surrounds a peripheral nerve containing the sensory neurons innervating the body region of the subject with the motor impairment due to SMA.

Embodiment 63. The electrical stimulus for use of any one of Embodiments 45-56, wherein the one or more electrodes penetrate a peripheral nerve or dorsal root ganglion containing the sensory neurons innervating the body region of the subject with the motor impairment due to SMA.

Embodiment 64. The electrical stimulus for use of any one of the prior Embodiments, wherein the body region of the subject is selected from at least one of lower back, hip, leg, ankle, and foot; and the one or more electrodes are implanted at the dorsolateral aspect of the spinal cord and span one or more of the T11-S1 nerve roots.

Embodiment 65. The electrical stimulus for use of any one of the prior Embodiments, wherein the body region of the subject is selected from at least one of upper arm, shoulder, arm, hand, and respiratory muscles; and the one or more electrodes are implanted at the dorsolateral aspect of the spinal cord and span one or more of the C3-T2 nerve roots.

Embodiment 66. The electrical stimulus for use of any one of the prior Embodiments, wherein the body region of the subject is selected from at least one of the chest, chest wall, abdomen, upper back, and middle back; and the one or more electrodes are implanted at the dorsolateral aspect of the spinal cord and span one or more of the T3-T10 nerve roots.

Embodiment 67. The electrical stimulus for use of any one of the prior Embodiments, wherein the neurostimulator is an external or implanted pulse generator.

Embodiment 68. The electrical stimulus for use of any one of the prior Embodiments, wherein the neurostimulator is implanted in the subject.

Embodiment 69. The electrical stimulus for use of any one of the prior Embodiments, wherein the subject with SMA is selected for treatment.

Embodiment 70. The electrical stimulus for use of any one of the prior Embodiments, wherein the motor impairment comprises partial or complete paralysis, loss of dexterity, loss of muscle strength, and/or uncontrollable muscle tone.

Embodiment 71. The electrical stimulus for use of any one of the prior Embodiments, wherein the locations of the one or more electrodes are chosen based on the body region of the subject with the motor impairment due to SMA.

Embodiment 72. The electrical stimulus for use of Embodiment 71, wherein the locations of the one or more electrodes are determined by: stimulating a plurality of contact points in the subject to target at least one muscle within the body region of the subject with the motor impairment due to SMA; measuring the electrical activity associated with said muscle in response to the stimulation; and identifying one or more contact points in the plurality of contact points as locations of the electrodes based on said electrical activity.

Embodiment 73. The electrical stimulus for use of any one of the prior Embodiments, wherein at least one of the one or more electrodes is placed transcutaneously.

Embodiment 74. The electrical stimulus for use of any one of the prior Embodiments, wherein at least one of the one or more electrodes is implanted.

Embodiment 75. Use of one or more electrodes controlled by a neurostimulator in the manufacture of a medicament for treating SMA in a subject, wherein the one or more electrodes controlled by the neurostimulator are configured to apply a therapeutically effective amount of an electrical stimulus to sensory neurons innervating a body region of the subject with a motor impairment due to SMA.

Embodiment 76. The use of Embodiment 75, wherein applying the electrical stimulus increases the firing rate probability of spinal motoneurons innervating the body region of the subject with the motor impairment due to SMA.

Embodiment 77. The use of Embodiment 75 or Embodiment 76, wherein the stimulation is applied at or below a motor threshold such that the stimulation does not directly elicit movement and/or muscle activity of the body region of the subject with the motor impairment due to SMA.

Embodiment 78. The use of Embodiments 75-77, wherein the electrical stimulus comprises electrical pulses having an amplitude of about 10 μA to about 100 mA, a width between about 40 μs and about 2 ms, and a frequency of about 10 Hz to about 2000 Hz.

Embodiment 79. The use of Embodiments 75-78, wherein the electrical stimulus comprises electrical pulses having an amplitude of about 10 μA to about 10 mA, a width between about 40 μs and about 2 ms, and a frequency of about 10 Hz to about 1000 Hz.

Embodiment 80. The use of Embodiments 75-79, wherein the electrical stimulus comprises electrical pulses having an amplitude of about 100 μA to about 10 mA, a width between about 40 μs and about 500 μs, and a frequency of about 10 Hz to about 1000 Hz.

Embodiment 81. The use of any one of the prior Embodiments, wherein the electrical stimulus comprises a stimulation pattern of a series of 2 to 5 pulses separated by inter-pulse intervals of about 3 ms to about 10 ms and wherein the series is repeated at a frequency of about 10 Hz to about 100 Hz.

Embodiment 82. The use of Embodiment 81, wherein the stimulation pattern is a series of 3 pulses separated by inter-pulse intervals of about 5 ms, wherein the series is repeated at a frequency of about 30 to about 100 Hz, and wherein the pulse width is about 200 μs.

Embodiment 83. The use of any one of Embodiments 78-82, wherein the pulses are cathodic-first biphasic or monophasic charge balanced pulses.

Embodiment 84. The use of any one of the prior Embodiments, wherein the electrical stimulus is applied for at least 2 hours/day over a period of at least 6 months.

Embodiment 85. The use of any one of Embodiments 75-83, wherein the electrical stimulus is applied for at least 1 hour/day over a period of at least 1 month.

Embodiment 86. The use of any one of the prior Embodiments, wherein the one or more electrodes are contained within an array of independently controllable electrodes implanted in the subject.

Embodiment 87. The use of Embodiment 86, wherein the electrode array is a multi-electrode paddle array.

Embodiment 88. The use of any one of the prior Embodiments, wherein the one or more electrodes are implanted epidurally at the spinal cord of the subject.

Embodiment 89. The use of any one of Embodiments 75-88, wherein the one or more electrodes are implanted at the dorsal rootlets of the one or more sensory neurons innervating the body region with the motor impairment of the subject.

Embodiment 90. The use of any one of Embodiments 75-88, wherein the one or more electrodes are implanted at the dorsolateral aspect of the spinal cord adjacent to the dorsal roots of the one or more sensory neurons innervating the body region with the motor impairment of the subject.

Embodiment 91. The use of any one of Embodiments 75-87, wherein the one or more electrodes are implanted at the dorsal root ganglia of the one or more sensory neurons innervating the body region with the motor impairment of the subject.

Embodiment 92. The use of any one of Embodiments 75-86, wherein the one or more electrodes are contained in a cuff that surrounds a peripheral nerve containing the sensory neurons innervating the body region of the subject with the motor impairment due to SMA.

Embodiment 93. The use of any one of Embodiments 75-86, wherein the one or more electrodes penetrate a peripheral nerve or dorsal root ganglion containing the sensory neurons innervating the body region of the subject with the motor impairment due to SMA.

Embodiment 94. The use of any one of the prior Embodiments, wherein the body region of the subject is selected from at least one of lower back, hip, leg, ankle, and foot; and the one or more electrodes are implanted at the dorsolateral aspect of the spinal cord and span one or more of the T11-S1 nerve roots.

Embodiment 95. The use of any one of the prior Embodiments, wherein the body region of the subject is selected from at least one of upper arm, shoulder, arm, hand, and respiratory muscles; and the one or more electrodes are implanted at the dorsolateral aspect of the spinal cord and span one or more of the C3-T2 nerve roots.

Embodiment 96. The use of any one of the prior Embodiments, wherein the body region of the subject is selected from at least one of the chest, chest wall, abdomen, upper back, and middle back; and the one or more electrodes are implanted at the dorsolateral aspect of the spinal cord and span one or more of the T3-T10 nerve roots.

Embodiment 97. The use of any one of the prior Embodiments, wherein the neurostimulator is an external or implanted pulse generator.

Embodiment 98. The use of any one of the prior Embodiments, wherein the neurostimulator is implanted in the subject.

Embodiment 99. The use of any one of the prior Embodiments, wherein the subject with SMA is selected for treatment.

Embodiment 100. The use of any one of the prior Embodiments, wherein the motor impairment comprises partial or complete paralysis, loss of dexterity, loss of muscle strength, and/or uncontrollable muscle tone.

Embodiment 101. The use of any one of the prior Embodiments, wherein the locations of the one or more electrodes are chosen based on the body region of the subject with the motor impairment due to SMA.

Embodiment 102. The use of Embodiment 101, wherein the locations of the one or more electrodes are determined by: stimulating a plurality of contact points in the subject to target at least one muscle within the body region of the subject with the motor impairment due to SMA; measuring the electrical activity associated with said muscle in response to the stimulation; and identifying one or more contact points in the plurality of contact points as locations of the electrodes based on said electrical activity.

Embodiment 103. The use of any one of the prior Embodiments, wherein at least one of the one or more electrodes is placed transcutaneously.

Embodiment 104. The use of any one of the prior Embodiments, wherein at least one of the one or more electrodes is implanted.

Embodiment 105. Use of one or more electrodes controlled by a neurostimulator in the manufacture of a medicament for treating SMA in a subject, wherein an electrical stimulus is to be applied using the one or more electrodes controlled by the neurostimulator to sensory neurons innervating a body region of the subject with a motor impairment due to SMA.

Embodiment 106. The use of Embodiment 105, wherein applying the electrical stimulus increases the firing rate probability of spinal motoneurons innervating the body region of the subject with the motor impairment due to SMA.

Embodiment 107. The use of Embodiment 105 or Embodiment 106, wherein the stimulation is applied at or below a motor threshold such that the stimulation does not directly elicit movement and/or muscle activity of the body region of the subject with the motor impairment due to SMA.

Embodiment 108. The use of Embodiments 105-107, wherein the electrical stimulus comprises electrical pulses having an amplitude of about 10 μA to about 100 mA, a width between about 40 μs and about 2 ms, and a frequency of about 10 Hz to about 2000 Hz.

Embodiment 109. The use of Embodiments 105-108, wherein the electrical stimulus comprises electrical pulses having an amplitude of about 10 μA to about 10 mA, a width between about 40 μs and about 2 ms, and a frequency of about 10 Hz to about 1000 Hz.

Embodiment 110. The use of Embodiments 105-109, wherein the electrical stimulus comprises electrical pulses having an amplitude of about 100 μA to about 10 mA, a width between about 40 μs and about 500 μs, and a frequency of about 10 Hz to about 1000 Hz.

Embodiment 111. The use of any one of the prior Embodiments, wherein the electrical stimulus comprises a stimulation pattern of a series of 2 to 5 pulses separated by inter-pulse intervals of about 3 ms to about 10 ms and wherein the series is repeated at a frequency of about 10 Hz to about 100 Hz.

Embodiment 112. The use of Embodiment 111, wherein the stimulation pattern is a series of 3 pulses separated by inter-pulse intervals of about 5 ms, wherein the series is repeated at a frequency of about 30 to about 100 Hz, and wherein the pulse width is about 200 μs.

Embodiment 113. The use of any one of Embodiments 108-112, wherein the pulses are cathodic-first biphasic or monophasic charge balanced pulses.

Embodiment 114. The use of any one of the prior Embodiments, wherein the electrical stimulus is applied for at least 2 hours/day over a period of at least 6 months.

Embodiment 115. The use of any one of Embodiments 105-113, wherein the electrical stimulus is applied for at least 1 hour/day over a period of at least 1 month.

Embodiment 116. The use of any one of the prior Embodiments, wherein the one or more electrodes are contained within an array of independently controllable electrodes implanted in the subject.

Embodiment 117. The use of Embodiment 116, wherein the electrode array is a multi-electrode paddle array.

Embodiment 118. The use of any one of the prior Embodiments, wherein the one or more electrodes are implanted epidurally at the spinal cord of the subject.

Embodiment 119. The use of any one of Embodiments 105-118, wherein the one or more electrodes are implanted at the dorsal rootlets of the one or more sensory neurons innervating the body region with the motor impairment of the subject.

Embodiment 120. The use of any one of Embodiments 105-118, wherein the one or more electrodes are implanted at the dorsolateral aspect of the spinal cord adjacent to the dorsal roots of the one or more sensory neurons innervating the body region with the motor impairment of the subject.

Embodiment 121. The use of any one of Embodiments 105-117, wherein the one or more electrodes are implanted at the dorsal root ganglia of the one or more sensory neurons innervating the body region with the motor impairment of the subject.

Embodiment 122. The use of any one of Embodiments 105-116, wherein the one or more electrodes are contained in a cuff that surrounds a peripheral nerve containing the sensory neurons innervating the body region of the subject with the motor impairment due to SMA.

Embodiment 123. The use of any one of Embodiments 105-116, wherein the one or more electrodes penetrate a peripheral nerve or dorsal root ganglion containing the sensory neurons innervating the body region of the subject with the motor impairment due to SMA.

Embodiment 124. The use of any one of the prior Embodiments, wherein the body region of the subject is selected from at least one of lower back, hip, leg, ankle, and foot; and the one or more electrodes are implanted at the dorsolateral aspect of the spinal cord and span one or more of the T11-S1 nerve roots.

Embodiment 125. The use of any one of the prior Embodiments, wherein the body region of the subject is selected from at least one of upper arm, shoulder, arm, hand, and respiratory muscles; and the one or more electrodes are implanted at the dorsolateral aspect of the spinal cord and span one or more of the C3-T2 nerve roots.

Embodiment 126. The use of any one of the prior Embodiments, wherein the body region of the subject is selected from at least one of the chest, chest wall, abdomen, upper back, and middle back; and the one or more electrodes are implanted at the dorsolateral aspect of the spinal cord and span one or more of the T3-T10 nerve roots.

Embodiment 127. The use of any one of the prior Embodiments, wherein the neurostimulator is an external or implanted pulse generator.

Embodiment 128. The use of any one of the prior Embodiments, wherein the neurostimulator is implanted in the subject.

Embodiment 129. The use of any one of the prior Embodiments, wherein the subject with SMA is selected for treatment.

Embodiment 130. The use of any one of the prior Embodiments, wherein the motor impairment comprises partial or complete paralysis, loss of dexterity, loss of muscle strength, and/or uncontrollable muscle tone.

Embodiment 131. The use of any one of the prior Embodiments, wherein the locations of the one or more electrodes are chosen based on the body region of the subject with the motor impairment due to SMA.

Embodiment 132. The use of Embodiment 131, wherein the locations of the one or more electrodes are determined by: stimulating a plurality of contact points in the subject to target at least one muscle within the body region of the subject with the motor impairment due to SMA; measuring the electrical activity associated with said muscle in response to the stimulation; and identifying one or more contact points in the plurality of contact points as locations of the electrodes based on said electrical activity.

Embodiment 133. The use of any one of the prior Embodiments, wherein at least one of the one or more electrodes is placed transcutaneously.

Embodiment 134. The use of any one of the prior Embodiments, wherein at least one of the one or more electrodes is implanted.

Embodiment 135. The method of any one of Embodiments 1 to 44, wherein the subject is receiving an SMA therapy in conjunction with the electrical stimulus.

Embodiment 136. The method of Embodiment 135, wherein the SMA therapy is selected from the group consisting of a vector-based gene therapy delivering a copy of the SMN1 gene; an antisense oligonucleotide (ASO) therapy targeting the SMN2 gene; or a small molecule SMN2-splicing modifier.

Embodiment 137. The method of Embodiment 135 or 136, wherein the SMA therapy is Onasemnogene abeparvovec, Nusinersen, or Risdiplam.

Embodiment 138. The method of any one of Embodiments 135 to 137, wherein the subject was administered Onasemnogen abeparvovec within the 6 years preceding the first application of electrical stimulus.

Embodiment 139. The method of any one of Embodiments 135 to 137, wherein the subject was administered Onasemnogen abeparvovec at least 18 months prior to the first application of electrical stimulus.

Embodiment 140. The method of any one of Embodiments 135 to 137, wherein the subject is receiving Nusinersen according to the maintenance dosing schedule at the time of the electrical stimulus.

Embodiment 141. The method of any one of Embodiments 135 to 137 or 140, wherein the subject was first administered Nusinersen at least 6 months prior to the first application of electrical stimulus.

Embodiment 142. The method of any one of Embodiments 135 to 137, wherein the subject is receiving Risdiplam.

Embodiment 143. The method of any one of Embodiments 135 to 137 or 142, wherein the subject began administration of Risdiplam within 18 months prior to the first application of electrical stimulus.

Embodiment 144. The method of any one of Embodiments 135 to 137 or 142, wherein the subject has been administered Risdiplam for at least 1 year prior to the first application of the electrical stimulus.

Embodiment 145. The electrical stimulus for use of any one of Embodiments 45 to 74, wherein the subject is receiving an SMA therapy in conjunction with the electrical stimulus.

Embodiment 146. The electrical stimulus for use of Embodiment 145, wherein the SMA therapy is selected from the group consisting of a vector-based gene therapy delivering a copy of the SMN1 gene; an antisense oligonucleotide (ASO) therapy targeting the SMN2 gene; or a small molecule SMN2-splicing modifier.

Embodiment 147. The electrical stimulus for use of Embodiment 145 or 146, wherein the SMA therapy is Onasemnogene abeparvovec, Nusinersen, or Risdiplam.

Embodiment 148. The electrical stimulus for use of any one of Embodiments 145 to 147, wherein the subject was administered Onasemnogen abeparvovec within the 6 years preceding the first application of electrical stimulus.

Embodiment 149. The electrical stimulus for use of any one of Embodiments 145 to 147, wherein the subject was administered Onasemnogen abeparvovec at least 18 months prior to the first application of electrical stimulus.

Embodiment 150. The electrical stimulus for use of any one of Embodiments 145 to 147, wherein the subject is receiving Nusinersen according to the maintenance dosing schedule at the time of the electrical stimulus.

Embodiment 151. The electrical stimulus for use of any one of Embodiments 145 to 147 or 150, wherein the subject was first administered Nusinersen at least 6 months prior to the first application of electrical stimulus.

Embodiment 152. The electrical stimulus for use of any one of Embodiments 145 to 147, wherein the subject is receiving Risdiplam.

Embodiment 153. The electrical stimulus for use of any one of Embodiments 145 to 147 or 152, wherein the subject began administration of Risdiplam within 18 months prior to the first application of electrical stimulus.

Embodiment 154. The electrical stimulus for use of any one of Embodiments 145 to 147 or 152, wherein the subject has been administered Risdiplam for at least 1 year prior to the first application of the electrical stimulus.

Embodiment 155. The use of any one of Embodiments 75 to 134, wherein the subject is receiving an SMA therapy in conjunction with the electrical stimulus.

Embodiment 156. The use of Embodiment 155, wherein the SMA therapy is selected from the group consisting of a vector-based gene therapy delivering a copy of the SMN1 gene; an antisense oligonucleotide (ASO) therapy targeting the SMN2 gene; or a small molecule SMN2-splicing modifier.

Embodiment 157. The use of Embodiment 155 or 156, wherein the SMA therapy is Onasemnogene abeparvovec, Nusinersen, or Risdiplam.

Embodiment 158. The use of any one of Embodiments 155 to 157, wherein the subject was administered Onasemnogen abeparvovec within the 6 years preceding the first application of electrical stimulus.

Embodiment 159. The use of any one of Embodiments 155 to 157, wherein the subject was administered Onasemnogen abeparvovec at least 18 months prior to the first application of electrical stimulus.

Embodiment 160. The use of any one of Embodiments 155 to 157, wherein the subject is receiving Nusinersen according to the maintenance dosing schedule at the time of the electrical stimulus.

Embodiment 161. The use of any one of Embodiments 155 to 157 or 160, wherein the subject was first administered Nusinersen at least 6 months prior to the first application of electrical stimulus.

Embodiment 162. The use of any one of Embodiments 155 to 157, wherein the subject is receiving Risdiplam.

Embodiment 163. The use of any one of Embodiments 155 to 157 or 162, wherein the subject began administration of Risdiplam within 18 months prior to the first application of electrical stimulus.

Embodiment 164. The use of any one of Embodiments 155 to 157 or 162, wherein the subject has been administered Risdiplam for at least 1 year prior to the first application of the electrical stimulus.

Embodiment 165. The method of any one of Embodiments 1 to 44, or 135 to 144, wherein the subject has type 1 SMA.

Embodiment 166. The method of any one of Embodiments 1 to 44, or 135 to 144, wherein the subject has type 2 SMA.

Embodiment 167. The method of any one of Embodiments 1 to 44, or 135 to 144, wherein the subject has type 3 SMA.

Embodiment 168. The method of any one of Embodiments 1 to 44, or 135 to 144, wherein the subject has type 4 SMA.

Embodiment 169. The electrical stimulus for use of any one of Embodiments 45 to 74, or 145 to 154, wherein the subject has type 1 SMA.

Embodiment 170. The electrical stimulus for use of any one of Embodiments 45 to 74, or 145 to 154, wherein the subject has type 2 SMA.

Embodiment 171. The electrical stimulus for use of any one of Embodiments 45 to 74, or 145 to 154, wherein the subject has type 3 SMA.

Embodiment 172. The electrical stimulus for use of any one of Embodiments 45 to 74, or 145 to 154, wherein the subject has type 4 SMA.

Embodiment 173. The use of any one of Embodiments 75 to 134, or 155 to 164, wherein the subject has type 1 SMA.

Embodiment 174. The use of any one of Embodiments 75 to 134, or 155 to 164, wherein the subject has type 2 SMA.

Embodiment 175. The use of any one of Embodiments 75 to 134, or 155 to 164, wherein the subject has type 3 SMA.

Embodiment 176. The use of any one of Embodiments 75 to 134, or 155 to 164, wherein the subject has type 4 SMA.

Embodiment 177. A method for treating spinal muscular atrophy (SMA) in a subject, comprising:
applying a therapeutically effective amount of an electrical stimulus to one or more sensory neurons innervating a body region of the subject with a motor impairment due to SMA;
wherein the subject is receiving an SMA therapy in conjunction with the electrical stimulus.

Embodiment 178. A method of stimulating one or more motoneurons impaired by SMA, said method comprising:
applying an electrical stimulus to at least one of said motoneurons, wherein said motoneurons innervate a body region of a subject with a motor impairment due to SMA;
wherein the subject is receiving an SMA therapy in conjunction with the electrical stimulus.

Embodiment 179. A method of increasing firing rate of one or more motoneurons impaired by SMA in a subject, comprising:
applying an electrical stimulus to one or more sensory neurons innervating a body region of the subject;
wherein the subject is receiving an SMA therapy in conjunction with the electrical stimulus;
thereby increasing the firing rate of the one or more motoneurons impaired by SMA in the subject.

Embodiment 180. The method of any one of Embodiments 177 to 179, wherein the SMA therapy is selected from the group consisting of a vector-based gene therapy delivering a copy of the SMN1 gene; an antisense oligonucleotide (ASO) therapy targeting the SMN2 gene; or a small molecule SMN2-splicing modifier.

Embodiment 181. The method of any one of Embodiments 177 to 180, wherein the SMA therapy is Onasemnogene abeparvovec, Nusinersen, or Risdiplam.

Embodiment 182. The method of any one of Embodiments 177 to 181, wherein the body region of the subject is selected from at least one of lower back, hip, leg, ankle, and foot.

Embodiment 183. The method of any one of Embodiments 177 to 182, wherein one or more electrodes are implanted at the dorsolateral aspect of the spinal cord and span one or more of the T11-S1 nerve roots; and the electrical stimulus is provided by one or more of the one or more electrodes.

Embodiment 184. The method of any one of Embodiments 177 to 180, wherein the body region of the subject is selected from at least one of upper arm, shoulder, arm, hand, and respiratory muscles.

Embodiment 185. The method of any one of Embodiments 177 to 180 or 184, wherein one or more electrodes are implanted at the dorsolateral aspect of the spinal cord and span one or more of the C3-T2 nerve roots; and the electrical stimulus is provided by one or more of the one or more electrodes.

Embodiment 186. The method of any one of Embodiments 177 to 180, wherein the body region of the subject is selected from at least one of the chest, chest wall, abdomen, upper back, and middle back.

Embodiment 187. The method of any one of Embodiments 177 to 180 or 186, wherein one or more electrodes are implanted at the dorsolateral aspect of the spinal cord and span one or more of the T3-T10 nerve roots; and the electrical stimulus is provided by one or more of the one or more electrodes.

Embodiment 188. The method of any one of Embodiments 177 to 187, wherein the firing rate probability of spinal motoneurons innervating the body region of the subject with the motor impairment due to SMA is increased.

Embodiment 189. The method of any one of Embodiments 177 to 188, wherein joint torque and muscle strength of the subject within the body region is increased.

Embodiment 190. The method of any one of Embodiments 177 to 189, wherein the electrical stimulus is applied for at least 1 hour/day over a period of at least 1 month.

Embodiment 191. The method of any one of Embodiments 177 to 190, wherein the stimulation is applied at or below a motor threshold such that the stimulation does not directly elicit movement and/or muscle activity of the body region of the subject with the motor impairment due to SMA.

Embodiment 192. The method of any one of Embodiments 177 to 191, wherein the electrical stimulus comprises electrical pulses having an amplitude of about 10 μA to about 100 mA, a width between about 40 μs and about 2 ms, and a frequency of about 10 Hz to about 2000 Hz.

Embodiment 193. The method of any one of claims Embodiments 177 to 192, wherein the motor impairment comprises partial or complete paralysis, loss of dexterity, loss of muscle strength, and/or uncontrollable muscle tone.

Embodiment 194. The method of any one of Embodiments 177 to 193, wherein the subject has type 1 SMA.

Embodiment 195. The method of any one of Embodiments 177 to 194, wherein the subject has type 2 SMA.

Embodiment 196. The method of any one of Embodiments 177 to 194, wherein the subject has type 3 SMA.

Embodiment 197. The method of any one of Embodiments 177 to 194, wherein the subject has type 4 SMA.

Embodiment 198. The method of any one of Embodiments 177 to 197, wherein the SMA therapy is Nusinersen.

Embodiment 199. The method of any one of Embodiments 177 to 198, wherein the SMA therapy is Risdiplam.

Embodiment 200. An electrical stimulus for use in treating spinal muscular atrophy (SMA) in a subject, wherein a therapeutically effective amount of the electrical stimulus is applied to one or more sensory neurons innervating a body region of the subject with a motor impairment due to SMA; and wherein the subject is receiving an SMA therapy in conjunction with the electrical stimulus.

Embodiment 201. An electrical stimulus for use in stimulating one or more motoneurons impaired by SMA, wherein the electrical stimulus is applied to at least one of said motoneurons, wherein said motoneurons innervate a body region of a subject with a motor impairment due to SMA; and wherein the subject is receiving an SMA therapy in conjunction with the electrical stimulus.

Embodiment 202. An electrical stimulus for use in increasing the firing rate of one or more motoneurons impaired by SMA in a subject, wherein the electrical stimulus is applied to one or more sensory neurons innervating a body region of the subject; wherein the subject is receiving an SMA therapy in conjunction with the electrical stimulus; thereby increasing the motoneuron firing rate of the subject.

Embodiment 203. The electrical stimulus for use of any one of Embodiments 200 to 202, wherein the SMA therapy is selected from the group consisting of a vector-based gene therapy delivering a copy of the SMN1 gene; an antisense oligonucleotide (ASO) therapy targeting the SMN2 gene; or a small molecule SMN2-splicing modifier.

Embodiment 204. The electrical stimulus for use of any one of Embodiments 200 to 203, wherein the SMA therapy is Onasemnogene abeparvovec, Nusinersen, or Risdiplam.

Embodiment 205. The electrical stimulus for use of any one of Embodiments 200 to 204, wherein the body region of the subject is selected from at least one of lower back, hip, leg, ankle, and foot.

Embodiment 206. The electrical stimulus for use of any one of Embodiments 200 to 205, wherein the electrical stimulus is provided by one or more electrodes implanted at the dorsolateral aspect of the spinal cord and spanning one or more of the T11-S1 nerve roots.

Embodiment 207. The electrical stimulus for use of any one of Embodiments 200 to 204, wherein the body region of the subject is selected from at least one of upper arm, shoulder, arm, hand, and respiratory muscles.

Embodiment 208. The electrical stimulus for use of any one of Embodiments 200 to 204 or 207, wherein the electrical stimulus is provided by one or more electrodes implanted at the dorsolateral aspect of the spinal cord and spanning one or more of the C3-T2 nerve roots.

Embodiment 209. The electrical stimulus for use of any one of Embodiments 200 to 204, wherein the body region of the subject is selected from at least one of the chest, chest wall, abdomen, upper back, and middle back.

Embodiment 210. The electrical stimulus for use of any one of Embodiments 200 to 204 or 209, wherein the electrical stimulus is provided by one or more electrodes implanted at the dorsolateral aspect of the spinal cord and spanning one or more of the T3-T10 nerve roots.

Embodiment 211. The electrical stimulus for use of any one of Embodiments 200 to 210, wherein the firing rate probability of spinal motoneurons innervating the body region of the subject with the motor impairment due to SMA is increased.

Embodiment 212. The electrical stimulus for use of any one of Embodiments 200 to 211, wherein joint torque and muscle strength of the subject within the body region is increased.

Embodiment 213. The electrical stimulus for use of any one of Embodiments 200 to 212, wherein the electrical stimulus is applied for at least 1 hour/day over a period of at least 1 month.

Embodiment 214. The electrical stimulus for use of any one of Embodiments 200 to 213, wherein the stimulation is applied at or below a motor threshold such that the stimulation does not directly elicit movement and/or muscle activity of the body region of the subject with the motor impairment due to SMA.

Embodiment 215. The electrical stimulus for use of any one of Embodiments 200 to 214, wherein the electrical stimulus comprises electrical pulses having an amplitude of about 10 μA to about 100 mA, a width between about 40 μs and about 2 ms, and a frequency of about 10 Hz to about 2000 Hz.

Embodiment 216. The electrical stimulus for use of any one of Embodiments 200 to 215, wherein the motor impairment comprises partial or complete paralysis, loss of dexterity, loss of muscle strength, and/or uncontrollable muscle tone.

Embodiment 217. The electrical stimulus for use of any one of Embodiments 200 to 216, wherein the subject has type 1 SMA.

Embodiment 218. The electrical stimulus for use of any one of Embodiments 200 to 216, wherein the subject has type 2 SMA.

Embodiment 219. The electrical stimulus for use of any one of Embodiments 200 to 216, wherein the subject has type 3 SMA.

Embodiment 220. The electrical stimulus for use of any one of Embodiments 200 to 216, wherein the subject has type 4 SMA.

Embodiment 221. The electrical stimulus for use of any one of Embodiments 200 to 220, wherein the SMA therapy is Nusinersen.

Embodiment 222. The electrical stimulus for use of any one of Embodiments 200 to 220, wherein the SMA therapy is Risdiplam.

Embodiment 223. An electrode assemblage comprising one or more electrodes configured to provide the electrical stimulus for use in any one of Embodiments 200 to 222.

Embodiment 224. The electrode assemblage of Embodiment 223, wherein the one or more electrodes comprise a multi-electrode paddle array.

Embodiment 224. The electrode assemblage of Embodiment 222 or 223, further comprising a neurostimulator controlling the one or more electrodes.

Embodiment 225. The electrode assemblage of any one of Embodiments 222 to 224, wherein the one or more electrodes are implanted epidurally at the spinal cord of the subject.

Embodiment 226. The electrode assemblage of any one of claims 222 to 225, wherein the one or more electrodes are implanted at the dorsal rootlets of the one or more sensory neurons innervating the body region with the motor impairment of the subject.

The foregoing description, for purpose of explanation, has been described with reference to specific examples. However, the illustrative discussions above are not intended to be exhaustive or to limit the invention to the precise forms disclosed. Many modifications and variations are possible in view of the above teachings. The examples were chosen and described in order to best explain the principles of the techniques and their practical applications. Others skilled in the art are thereby enabled to best utilize the techniques and various examples with various modifications as are suited to the particular use contemplated.

It will be apparent that the precise details of the methods or compositions described may be varied or modified without departing from the spirit of the described implementations. All such modifications and variations that fall within the scope and spirit of the claims below are claimed.

It is claimed:

1. A method for treating spinal muscular atrophy (SMA) in a subject, comprising:
applying a therapeutically effective amount of an electrical stimulus to one or more sensory neurons innervating a body region of the subject with a motor impairment due to SMA;
wherein the subject is receiving an SMA therapy in conjunction with the electrical stimulus.

2. A method of stimulating one or more motoneurons impaired by SMA, said method comprising:
applying an electrical stimulus to at least one of said motoneurons, wherein said motoneurons innervate a body region of a subject with a motor impairment due to SMA;
wherein the subject is receiving an SMA therapy in conjunction with the electrical stimulus.

3. A method of increasing firing rate of one or more motoneurons impaired by SMA in a subject, comprising:
applying an electrical stimulus to one or more sensory neurons innervating a body region of the subject;
wherein the subject is receiving an SMA therapy in conjunction with the electrical stimulus;
thereby increasing the firing rate of the one or more motoneurons impaired by SMA in the subject.

4. The method of claim 1, wherein the SMA therapy is selected from the group consisting of a vector-based gene therapy delivering a copy of the SMN1 gene; an antisense oligonucleotide (ASO) therapy targeting the SMN2 gene; or a small molecule SMN2-splicing modifier.

5. The method of claim 1, wherein the SMA therapy is Onasemnogene abeparvovec, Nusinersen, or Risdiplam.

6. The method of claim 1, wherein the body region of the subject is selected from at least one of lower back, hip, leg, ankle, and foot.

7. The method of claim 1, wherein one or more electrodes are implanted at the dorsolateral aspect of the spinal cord and span one or more of the T11-S1 nerve roots; and the electrical stimulus is provided by one or more of the one or more electrodes.

8. The method of claim 1, wherein the body region of the subject is selected from at least one of upper arm, shoulder, arm, hand, and respiratory muscles.

9. The method of claim 1, wherein one or more electrodes are implanted at the dorsolateral aspect of the spinal cord and span one or more of the C3-T2 nerve roots; and the electrical stimulus is provided by one or more of the one or more electrodes.

10. The method of claim 1, wherein the body region of the subject is selected from at least one of the chest, chest wall, abdomen, upper back, and middle back.

11. The method of claim 1, wherein one or more electrodes are implanted at the dorsolateral aspect of the spinal cord and span one or more of the T3-T10 nerve roots; and the electrical stimulus is provided by one or more of the one or more electrodes.

12. The method of claim 1, wherein the firing rate probability of spinal motoneurons innervating the body region of the subject with the motor impairment due to SMA is increased.

13. The method of claim 1, wherein joint torque and muscle strength of the subject within the body region is increased.

14. The method of claim 1, wherein the electrical stimulus is applied for at least 1 hour/day over a period of at least 1 month.

15. The method of claim 1, wherein the stimulation is applied at or below a motor threshold such that the stimulation does not directly elicit movement and/or muscle activity of the body region of the subject with the motor impairment due to SMA.

16. The method of claim 1, wherein the electrical stimulus comprises electrical pulses having an amplitude of about 10 μA to about 100 mA, a width between about 40 μs and about 2 ms, and a frequency of about 10 Hz to about 2000 Hz.

17. The method of claim 1, wherein the motor impairment comprises partial or complete paralysis, loss of dexterity, loss of muscle strength, and/or uncontrollable muscle tone.

18. The method of claim 1, wherein the subject has type 1 SMA, type 2 SMA, type 3 SMA, or type 4 SMA.

19. The method of claim 2, wherein the SMA therapy is Nusinersen or Risdiplam.

20. The method of claim 3, wherein the SMA therapy is Nusinersen or Risdiplam.
